SEQUENCE LISTING (170 sequences)

SEQ ID NO: 1 - protein, 124 aa, synthetic construct
EVQLQESGGGLVQAGGSLRLSCAASGGTFSYYNMGWFRQAPGKEREFVAVISQYGSNTYYADSVKGRFTITRDNAKNAAYLQLNSLEPEDTAVYYCATLRSGSDWRTIRDYDNWGQGTQVTVSS

SEQ ID NO: 2 - protein, 115 aa, synthetic construct
EVQLQESGGGLVQPGGSLRLSCAASGDIFSFNVMGWFRQAPGKERELVATIAPGGNTNYFDSARGRFTISRDSAKNTVYLQMNSLKPEDTAVYYCNRWPFSRDSWGQGTQVTVSS

SEQ ID NO: 3 - protein, 118 aa, synthetic construct
EVQLQESGGGLVQAGGSLRLSCVASGMAFGSSPMNWVRQAPGKEPEWVSGVTSAGYAYYADWVKGRFTASRDNAKNTLFLQMNSLKPEDTAVYYCARGPFHSTAYSTRGQGTQVTVSS

SEQ ID NO: 4 - protein, 118 aa, synthetic construct
EVQLQESGGGLVQPGDSLRLSCVGSGLTFGSSLMNWVRQAPGKGPEWVGGVTSGGYSYYAESVKGRFTISRDNAKNTLFLQMNSLKPEDTAVYYCARGPFHSIAYATRGQGTQVTVSS

SEQ ID NO: 5 - protein, 118 aa, synthetic construct
EVQLQESGGGLVQPGDSLRLSCVGSGFTFGSTLMNWVRQAPGKGPEWVGGVTSGGYSYYADSVKGRFTISRDNAKNTLFLQMNSLKPEDTAVYYCARGPFHSIAYATRGQGTQVTVSS

SEQ ID NO: 6 - protein, 124 aa, synthetic construct
EVQLQESGGGLVQAGGSLRLSCAASGLTFSYYNMGWFRQAPGKEREFVAVISQYGSNTYYADSVKGRFTITRDNAKNAAYLQLNSLKPEDTAVYYCATLRSGSDWRTMRDYDNWGQGTQVTVSS

SEQ ID NO: 7 - protein, 115 aa, synthetic construct
EVQLQESGGGLVQPGGSLRLSCAASGNIFSFNVMGWFRQAPGKERELVATIAPGGKTNYADPAKGRFTISRDSAKNTVYLQMNSLKPEDTAVYYCNRWPFSRDSWGQGTQVTVSS

SEQ ID NO: 8 - protein, 118 aa, synthetic construct
EVQLQESGGGLVQAGGSLRLSCAASGMAFGSSPMNWVRQAPGKEPEWVSGVTSAGYAYYADWVKGRFTASRDNAKNTLFLQMNSLKPEDTAVYYCARGPFHSTAYSTRGQGTQVTVSS

SEQ ID NO: 9 - protein, 115 aa, synthetic construct
EVQLQESGGGLVQPGGSLRLSCASSGNIFSFNVMGWFRQAPGKERELVATIAPGGKTNYADPAKGRFTISRDSAKNTVYLQMNSLKPEDTAVYYCNRWPFSRDSWGQGTQVTVSS

SEQ ID NO: 10 - protein, 115 aa, synthetic construct
EVQLQESGGGLVQPGGSLRLSCTASGNIFNINVMGWYRQAPGKERELLAHITSGGRVTYFDSAKGRFTITRDNAKNTMYLQMNSLKPEDTAVYYCNTLVHRRDSWGQGTQVTVSS

SEQ ID NO: 11 - protein, 115 aa, synthetic construct
EVQLQESGGGLMQAGGSLRLSCAASGDIFSFNVMGWFRQAPGKERELVATIAPGGNTNYFDSARGRFTISRDSAKNTVYLQMNSLKPEDTAVYYCNRWPFSRDSWGQGTQVTVSS

SEQ ID NO: 12 - protein, 113 aa, synthetic construct
EVQLQESGGGLVQPGGSLRLSCAASKSTFSGNVIAWYRQAPGKQRELIAYITSGGKTLYADSAKGRFTISRDNAKNTVSLQMNSLEPEDTAVYYCNTLPFKDWGQGTQVTVSS

SEQ ID NO: 13 - protein, 115 aa, synthetic construct
EVQLQESGGGLVQPGGSLRLSCAASGDIFSFNVMGWFRQAPGKERELVATIAPGGKTNYADPAKGRFTISRDSAKNTVYLQMNSLKPEDTAVYYCNRWPFSRDSWGQGTQVTVSS

SEQ ID NO: 14 - protein, 115 aa, synthetic construct
EVQLQESGGGLVQPGGSLRLSCAASENIFRSTVMGWYRQAPGKERELLAHITSGGRAQYFDSAKGRFTITRDNAKNTVFLQMNSLKPEDTAVYYCNSLVHRRDSWGQGTQVTVSS

SEQ ID NO: 15 - protein, 118 aa, synthetic construct
EVQLQESGGGLVQPGGSLRLSCAASGFTFGSTTMNWVRQAPGKGPEWVGGVTSSGYSYYADSVKGRFTISRDNAKNTLFLQMNSLKFEDTAVYYCARGPFHSTAYATRGQGTQVTVSS

SEQ ID NO: 16 - protein, 115 aa, synthetic construct
EVQLQESGGGLVQAGGSLRLSCAASGMSFGPNVIGWFRQAPGKQRELVAHITSGGNTLYADSAKGRFTISRDNAANTVYLQMNSLKSEDTAVYYCNTLPWTRGSWGQGTQVTVSS

SEQ ID NO: 17 - protein, 115 aa, synthetic construct
EVQLQESGGGLVQAGGSLRLSCAASGNTGRYNVFGWFRQAPGKQRELVAHITTGGRTFYSDAGKGRFTITRNNAENTVYLQMNSLKPEDTAVYYCNSLPFEAHSWGQGTQVTVSS

SEQ ID NO: 18 - protein, 115 aa, synthetic construct
EVQLQESGGGLVQPGGSLRLSCAASGNIFRSNVMGWYRQAPGKERELLAHITSGGKTQYFDSAKGRFTITRDNAKNTVYLQMNSLKPEDTAVYYCNSLVHRRDSWGQGTQVTVSS

SEQ ID NO: 19 - protein, 115 aa, synthetic construct
EVQLQESGGGLVQLGGSLRLSCAASGDIFSFNVMGWFRQAPGKERELVATIAPGGNTNYFDSARGRFTISRDSAKNTVYLQMNSLKPEDTAVYYCNRWPFSRDSWGQGTQVTVSS

SEQ ID NO: 20 - protein, 115 aa, synthetic construct
EVQLQESGGGLVQPGGSLRLSCTASRDIFSFNIMAWYRQAPGKERELVARITTGGITWYADPGQGRFTISRDNVENTVYLQMNSLKPEDTAVYYCNTLPFRPTYWGQGTQVTVSS

SEQ ID NO: 21 - protein, 115 aa, synthetic construct
EVQLQESGGGLVQPGGSLRLSCVASGNIFRSNVMGWYRQAPGKERELLAHITSDGRTQYFDSAKGRFTITRDNAKNTVYLQMNSLKPEDTAVYYCNSLVHRRDSWGQGTQVTVSS

SEQ ID NO: 22 - protein, 115 aa, synthetic construct
EVQLQESGGGLVQPGGSLRLSCVASGNIFRSNVMGWYRQAPGKERELLAHITSGGSTQYFDSAKGRFTITRDNAKNTVYLQMNSLKPEDTAIYYCNSLVHRRDSWGQGTQVTVSS

SEQ ID NO: 23 - protein, 115 aa, synthetic construct
EVQLQESGGGLVQPGGSLRLSCAAPRSIFVFNIMGWYRQAPGKERELVAHISSGGNTFYSSSVEGRFTISRDTAKNTVYLQMNSLKPEDTAVYYCNTLPFRPASWGQGTQVTVSS

SEQ ID NO: 24 - protein, 118 aa, synthetic construct
EVQLQESGGGLVQPGGSLRLSCAASGMAFGSSPMNWVRQAPGKGPEWVSGTTSAGYTYYADSVKGRFTASRDNAKNTLFLQMNSLEPEDTAVYYCARGPFHSTAYSTRGQGTQVTVSS

SEQ ID NO: 25 - protein, 114 aa, synthetic construct
EVQLQESGGGLVQPGGSLRLSCAASGSIFSGNVMGWFRQAPGKEREAVAWLTPDGSRTYYADSAKGRFTISRDNTKNTVYLQMNSLKPEDTAVYYCNKLPFSSWGQGTQVTVSS

SEQ ID NO: 26 - protein, 5 aa, synthetic construct
YYNMG

SEQ ID NO: 27 - protein, 5 aa, synthetic construct
FNVMG

SEQ ID NO: 28 - protein, 5 aa, synthetic construct
SSPMN

SEQ ID NO: 29 - protein, 5 aa, synthetic construct
SSLMN

SEQ ID NO: 30 - protein, 5 aa, synthetic construct
STLMN

SEQ ID NO: 31 - protein, 5 aa, synthetic construct
YYNMG

SEQ ID NO: 32 - protein, 5 aa, synthetic construct
FNVMG

SEQ ID NO: 33 - protein, 5 aa, synthetic construct
SSPMN

SEQ ID NO: 34 - protein, 5 aa, synthetic construct
FNVMG

SEQ ID NO: 35 - protein, 5 aa, synthetic construct
INVMG

SEQ ID NO: 36 - protein, 5 aa, synthetic construct
FNVMG

SEQ ID NO: 37 - protein, 5 aa, synthetic construct
GNVIA

SEQ ID NO: 38 - protein, 5 aa, synthetic construct
FNVMG

SEQ ID NO: 39 - protein, 5 aa, synthetic construct
STVMG

SEQ ID NO: 40 - protein, 5 aa, synthetic construct
STTMN

SEQ ID NO: 41 - protein, 5 aa, synthetic construct
PNVIG

SEQ ID NO: 42 - protein, 5 aa, synthetic construct
YNVFG

SEQ ID NO: 43 - protein, 5 aa, synthetic construct
SNVMG

SEQ ID NO: 44 - protein, 5 aa, synthetic construct
FNVMG

SEQ ID NO: 45 - protein, 5 aa, synthetic construct
FNIMA

SEQ ID NO: 46 - protein, 5 aa, synthetic construct
SNVMG

SEQ ID NO: 47 - protein, 5 aa, synthetic construct
SNVMG

SEQ ID NO: 48 - protein, 5 aa, synthetic construct
FNIMG

SEQ ID NO: 49 - protein, 5 aa, synthetic construct
SSPMN

SEQ ID NO: 50 - protein, 5 aa, synthetic construct
GNVMG

SEQ ID NO: 51 - protein, 17 aa, synthetic construct
VISQYGSNTYYADSVKG

SEQ ID NO: 52 - protein, 16 aa, synthetic construct
TIAPGGNTNYFDSARG

SEQ ID NO: 53 - protein, 16 aa, synthetic construct
GVTSAGYAYYADWVKG

SEQ ID NO: 54 - protein, 16 aa, synthetic construct
GVTSGGYSYYAESVKG

SEQ ID NO: 55 - protein, 16 aa, synthetic construct
GVTSGGYSYYADSVKG

SEQ ID NO: 56 - protein, 17 aa, synthetic construct
VISQYGSNTYYADSVKG

SEQ ID NO: 57 - protein, 16 aa, synthetic construct
TIAPGGKTNYADPAKG

SEQ ID NO: 58 - protein, 16 aa, synthetic construct
GVTSAGYAYYADWVKG

SEQ ID NO: 59 - protein, 16 aa, synthetic construct
TIAPGGKTNYADPAKG

SEQ ID NO: 60 - protein, 16 aa, synthetic construct
HITSGGRVTYFDSAKG

SEQ ID NO: 61 - protein, 16 aa, synthetic construct
TIAPGGNTNYFDSARG

SEQ ID NO: 62 - protein, 16 aa, synthetic construct
YITSGGKTLYADSAKG

SEQ ID NO: 63 - protein, 16 aa, synthetic construct
TIAPGGKTNYADPAKG

SEQ ID NO: 64 - protein, 16 aa, synthetic construct
HITSGGRAQYFDSAKG

SEQ ID NO: 65 - protein, 16 aa, synthetic construct
GVTSSGYSYYADSVKG

SEQ ID NO: 66 - protein, 16 aa, synthetic construct
HITSGGNTLYADSAKG

SEQ ID NO: 67 - protein, 16 aa, synthetic construct
HITTGGRTFYSDAGKG

SEQ ID NO: 68 - protein, 16 aa, synthetic construct
HITSGGKTQYFDSAKG

SEQ ID NO: 69 - protein, 16 aa, synthetic construct
TIAPGGNTNYFDSARG

SEQ ID NO: 70 - protein, 16 aa, synthetic construct
RITTGGITWYADPGQG

SEQ ID NO: 71 - protein, 16 aa, synthetic construct
HITSDGRTQYFDSAKG

SEQ ID NO: 72 - protein, 16 aa, synthetic construct
HITSGGSTQYFDSAKG

SEQ ID NO: 73 - protein, 16 aa, synthetic construct
HISSGGNTFYSSSVEG

SEQ ID NO: 74 - protein, 16 aa, synthetic construct
GTTSAGYTYYADSVKG

SEQ ID NO: 75 - protein, 17 aa, synthetic construct
WLTPDGSRTYYADSAKG

SEQ ID NO: 76 - protein, 15 aa, synthetic construct
LRSGSDWRTIRDYDN

SEQ ID NO: 77 - protein, 7 aa, synthetic construct
WPFSRDS

SEQ ID NO: 78 - protein, 10 aa, synthetic construct
GPFHSTAYST

SEQ ID NO: 79 - protein, 10 aa, synthetic construct
GPFHSIAYAT

SEQ ID NO: 80 - protein, 10 aa, synthetic construct
GPFHSIAYAT

SEQ ID NO: 81 - protein, 15 aa, synthetic construct
LRSGSDWRTMRDYDN

SEQ ID NO: 82 - protein, 7 aa, synthetic construct
WPFSRDS

SEQ ID NO: 83 - protein, 10 aa, synthetic construct
GPFHSTAYST

SEQ ID NO: 84 - protein, 7 aa, synthetic construct
WPFSRDS

SEQ ID NO: 85 - protein, 7 aa, synthetic construct
LVHRRDS

SEQ ID NO: 86 - protein, 7 aa, synthetic construct
WPFSRDS

SEQ ID NO: 87 - protein, 5 aa, synthetic construct
LPFKD

SEQ ID NO: 88 - protein, 7 aa, synthetic construct
WPFSRDS

SEQ ID NO: 89 - protein, 7 aa, synthetic construct
LVHRRDS

SEQ ID NO: 90 - protein, 10 aa, synthetic construct
GPFHSTAYAT

SEQ ID NO: 91 - protein, 7 aa, synthetic construct
LPWTRGS

SEQ ID NO: 92 - protein, 7 aa, synthetic construct
LPFEAHS

SEQ ID NO: 93 - protein, 7 aa, synthetic construct
LVHRRDS

SEQ ID NO: 94 - protein, 7 aa, synthetic construct
WPFSRDS

SEQ ID NO: 95 - protein, 7 aa, synthetic construct
LPFRPTY

SEQ ID NO: 96 - protein, 7 aa, synthetic construct
LVHRRDS

SEQ ID NO: 97 - protein, 7 aa, synthetic construct
LVHRRDS

SEQ ID NO: 98 - protein, 7 aa, synthetic construct
LPFRPAS

SEQ ID NO: 99 - protein, 10 aa, synthetic construct
GPFHSTAYST

SEQ ID NO: 100 - protein, 5 aa, synthetic construct
LPFSS

SEQ ID NO: 101 - protein, 124 aa, synthetic construct
QVQLVESGGGVVQPGRSLRLSCAASGGTFSYYNMGWFRQAPGKGREFVAVISQYGSNTYYADSVKGRFTISRDNSKNTLYLQMNSLRAEDTAVYYCATLRSGSDWRTIRDYDNWGQGTQVTVSS

SEQ ID NO: 102 - protein, 124 aa, synthetic construct
EVQLLESGGGLVQPGGSLRLSCAASGGTFSYYNMGWFRQAPGQGREFVAVISQYGSNTYYADSVKGRFTISRDNSKNTLYLQMNSLRAEDTAVYYCATLRSGSDWRTIRDYDNWGQGTLVTVSS

SEQ ID NO: 103 - protein, 124 aa, synthetic construct
EVQLQASGGGFVQPGGSLRLSCAASGGTFSYYNMGWFRQAPGKEREFVSVISQYGSNTYYADSVKGRFTISRDNSKNTVYLQMNSLRAEDTATYYCATLRSGSDWRTIRDYDNWGQGTQVTVSS

SEQ ID NO: 104 - protein, 124 aa, synthetic construct
EVQLVASGGGFVQPGGSLRLSCAASGGTFSYYNMGWFRQAPGKGREFVSVISQYGSNTYYADSVKGRFTISRDNSKNTVYLQMNSLRAEDTATYYCATLRSGSDWRTIRDYDNWGQGTLVTVSS

SEQ ID NO: 105 - protein, 115 aa, synthetic construct
EVQLVESGGGLVQPGGSLRLSCAASGDIFSFNVMGWFRQAPGKERELVSVIAPGGNTYYADSVKGRFTISRDNSKNTLYLQMNSLRAEDTAVYYCNRWPFSRDSWGQGTLVTVSS

SEQ ID NO: 106 - protein, 7 aa, synthetic construct
GGTFSYY

SEQ ID NO: 107 - protein, 7 aa, synthetic construct
GDIFSFN

SEQ ID NO: 108 - protein, 7 aa, synthetic construct
GMAFGSS

SEQ ID NO: 109 - protein, 6 aa, synthetic construct
SQYGSN

SEQ ID NO: 110 - protein, 5 aa, synthetic construct
APGGN

SEQ ID NO: 111 - protein, 5 aa, synthetic construct
TSAGY

SEQ ID NO: 112 - protein, 15 aa, synthetic construct
LRSGSDWRTIRDYDN

SEQ ID NO: 113 - protein, 7 aa, synthetic construct
WPFSRDS

SEQ ID NO: 114 - protein, 10 aa, synthetic construct
GPFHSTAYST

SEQ ID NO: 115 - protein, 8 aa, synthetic construct
GGTFSYYN

SEQ ID NO: 116 - protein, 8 aa, synthetic construct
GDIFSFNV

SEQ ID NO: 117 - protein, 8 aa, synthetic construct
GMAFGSSP

SEQ ID NO: 118 - protein, 8 aa, synthetic construct
ISQYGSNT

SEQ ID NO: 119 - protein, 7 aa, synthetic construct
IAPGGNT

SEQ ID NO: 120 - protein, 7 aa, synthetic construct
VTSAGYA

SEQ ID NO: 121 - protein, 17 aa, synthetic construct
ATLRSGSDWRTIRDYDN

SEQ ID NO: 122 - protein, 9 aa, synthetic construct
NRWPFSRDS

SEQ ID NO: 123 - protein, 12 aa, synthetic construct
ARGPFHSTAYST

SEQ ID NO: 124 - protein, 13 aa, synthetic construct
AASGGTFSYYNMG

SEQ ID NO: 125 - protein, 13 aa, synthetic construct
AASGDIFSFNVMG

SEQ ID NO: 126 - protein, 13 aa, synthetic construct
VASGMAFGSSPMN

SEQ ID NO: 127 - protein, 10 aa, synthetic construct
VISQYGSNTY

SEQ ID NO: 128 - protein, 9 aa, synthetic construct
VIAPGGNTY

SEQ ID NO: 129 - protein, 9 aa, synthetic construct
GVTSAGYAY

SEQ ID NO: 130 - protein, 17 aa, synthetic construct
ATLRSGSDWRTIRDYDN

SEQ ID NO: 131 - protein, 9 aa, synthetic construct
NRWPFSRDS

SEQ ID NO: 132 - protein, 12 aa, synthetic construct
ARGPFHSTAYST

SEQ ID NO: 133 - protein, 5 aa, synthetic construct
YYNMG

SEQ ID NO: 134 - protein, 5 aa, synthetic construct
FNVMG

SEQ ID NO: 135 - protein, 5 aa, synthetic construct
SSPMN

SEQ ID NO: 136 - protein, 17 aa, synthetic construct
VISQYGSNTYYADSVKG

SEQ ID NO: 137 - protein, 16 aa, synthetic construct
VIAPGGNTYYADSVKG

SEQ ID NO: 138 - protein, 16 aa, synthetic construct
GVTSAGYAYYADWVKG

SEQ ID NO: 139 - protein, 15 aa, synthetic construct
LRSGSDWRTIRDYDN

SEQ ID NO: 140 - protein, 7 aa, synthetic construct
WPFSRDS

SEQ ID NO: 141 - protein, 10 aa, synthetic construct
GPFHSTAYST

SEQ ID NO: 142 - protein, 972 aa, synthetic construct
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 143 - protein, 84 aa, synthetic construct
PVIEPSVPELVVKPGATVTLRCVGNGSVEWDGPPSPHWTLYSDGSSSILSTNNATFQNTGTYRCTEPGDPLGGSAAIHLYVKDP

SEQ ID NO: 144 - protein, 91 aa, synthetic construct
PWNVLAQEVVVFEDQDALLPCLLTDPVLEAGVSLVRVRGRPLMRHTNYSFSPWHGFTIHRAKFIQSQDYQCSALMGGRKVMSISIRLKVQK

SEQ ID NO: 145 - protein, 88 aa, synthetic construct
PALTLVPAELVRIRGEAAQIVCSASSVDVNFDVFLQHNNTKLAIPQQSDFHNNRYQKVLTLNLDQVDFQHAGNYSCVASNVQGKHSTS

SEQ ID NO: 146 - protein, 101 aa, synthetic construct
AYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLR

SEQ ID NO: 147 - protein, 8 aa, synthetic construct
  VARIANT 2: note = X = G or L or F
  VARIANT 6: note = X = S or Y
GXTFSXYN

SEQ ID NO: 148 - protein, 7 aa, synthetic construct
  VARIANT 2: note = X = F or M or L
  VARIANT 3: note = X = T or A
  VARIANT 7: note = X = T or S
GXXFGSX

SEQ ID NO: 149 - protein, 8 aa, synthetic construct
  VARIANT 2: note = X = D or N
  VARIANT 5: note = X = R or S
  VARIANT 6: note = X = F or S
  VARIANT 7: note = X = T or N
GXIFXXXV

SEQ ID NO: 150 - protein, 8 aa, synthetic construct
ISQYGSNT

SEQ ID NO: 151 - protein, 7 aa, synthetic construct
  VARIANT 1: note = X = V or T
  VARIANT 4: note = X = S or A or G
  VARIANT 7: note = X = S or A or T
XTSXGYX

SEQ ID NO: 152 - protein, 7 aa, synthetic construct
  VARIANT 2: note = X = T or A or S
  VARIANT 3: note = X = S or P or T
  VARIANT 6: note = X = R or N or K
IXXGGXT

SEQ ID NO: 153 - protein, 17 aa, synthetic construct
  VARIANT 11: note = X = I or M
TLRSGSDWRTXRDYDNW

SEQ ID NO: 154 - protein, 12 aa, synthetic construct
  VARIANT 7: note = X = T or I
  VARIANT 10: note = X = A or S
RGPFHSXAYXTR

SEQ ID NO: 155 - protein, 8 aa, synthetic construct
  VARIANT 1: note = X = R or T or S
  VARIANT 5: note = X = R or S
XLPFXRDS

SEQ ID NO: 156 - protein, 9 aa, synthetic construct
TIAPGGNTN

SEQ ID NO: 157 - protein, 16 aa, synthetic construct
TIAPGGNTNYFDSARG

SEQ ID NO: 158 - protein, 101 aa, synthetic construct
PEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPIS

SEQ ID NO: 159 - protein, 502 aa, synthetic construct
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFHHHHHHHH

SEQ ID NO: 160 - protein, 500 aa, synthetic construct
APVIEPSGPELVVEPGETVTLRCVSNGSVEWDGPISPYWTLDPESPGSTLTTRNATFKNTGTYRCTELEDPMAGSTTIHLYVKDPAHSWNLLAQEVTVVEGQEAVLPCLITDPALKDSVSLMREGGRQVLRKTVYFFSPWRGFIIRKAKVLDSNTYVCKTMVNGRESTSTGIWLKVNRVHPEPPQIKLEPSKLVRIRGEAAQIVCSATNAEVGFNVILKRGDTKLEIPLNSDFQDNYYKKVRALSLNAVDFQDAGIYSCVASNDVGTRTATMNFQVVESAYLNLTSEQSLLQEVSVGDSLILTVHADAYPSIQHYNWTYLGPFFEDQRKLEFITQRAIYRYTFKLFLNRVKASEAGQYFLMAQNKAGWNNLTFELTLRYPPEVSVTWMPVNGSDVLFCDVSGYPQPSVTWMECRGHTDRCDEAQALQVWNDTHPEVLSQKPFDKVIIQSQLPIGTLKHNMTYFCKTHNSVGNSSQYFRAVSLGQSKQLPDESHHHHHHHH

SEQ ID NO: 161 - protein, 500 aa, synthetic construct
APVIEPSGPELVVEPGETVTLRCVSNGSVEWDGPISPYWTLDPESPGSTLTTRNATFKNTGTYRCTELEDPMAGSTTIHLYVKDPAHSWNLLAQEVTVVEGQEAVLPCLITDPALKDSVSLMREGGRQVLRKTVYFFSPWRGFIIRKAKVLDSNTYVCKTMVNGRESTSTGIWLKVNRVHPEPPQIKLEPSKLVRIRGEAAQIVCSATNAEVGFNVILKRGDTKLEIPLNSDFQDNYYKKVRALSLNAVDFQDAGIYSCVASNDVGTRTATMNFQVVESAYLNLTSEQSLLQEVSVGDSLILTVHADAYPSIQHYNWTYLGPFFEDQRKLEFITQRAIYRYTFKLFLNRVKASEAGQYFLMAQNKAGWNNLTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFHHHHHHHH

SEQ ID NO: 162 - protein, 5 aa, synthetic construct
YYNMG

SEQ ID NO: 163 - skipped

SEQ ID NO: 164 - protein, 5 aa, synthetic construct
FNVMG

SEQ ID NO: 165 - protein, 17 aa, synthetic construct
VISQYGSNTYYADSVKG

SEQ ID NO: 166 - protein, 16 aa, synthetic construct
  VARIANT 2: note = X = V or T
  VARIANT 5: note = X = S or A or G
  VARIANT 8: note = X = S or A or T
  VARIANT 12: note = X = D or E
  VARIANT 13: note = X = S or W
GXTSXGYXYYAXXVKG

SEQ ID NO: 167 - protein, 16 aa, synthetic construct
  VARIANT 7: note = X = N or K
  VARIANT 11: note = X = F or A
  VARIANT 13: note = X = S or P
  VARIANT 15: note = X = R or K
TIAPGGXTNYXDXAXG

SEQ ID NO: 168 - protein, 15 aa, synthetic construct
  VARIANT 10: note = X = I or M
LRSGSDWRTXRDYDN

SEQ ID NO: 169 - protein, 10 aa, synthetic construct
  VARIANT 6: note = X = T or I
  VARIANT 9: note = X = A or S
GPFHSXAYXT

SEQ ID NO: 170 - protein, 7 aa, synthetic construct
WPFSRDS

(19) United States
(12) Patent Application Publication
(10) Pub. No.: US 2026/0308263 A1
(43) Pub. Date: Oct. 8, 2026

(54) COLONY STIMULATING FACTOR 1 RECEPTOR ANTIBODIES AND METHODS OF USE THEREOF

(71) Applicant: Otsuka Pharmaceutical Co., Ltd., Tokyo (JP)

(72) Inventors: Shingo Nishikori, Waltham, MA (US); Amyn Murji, Waltham, MA (US); Nicole Brossi, Waltham, MA (US); Karthik Viswanathan, Waltham, MA (US); Ramki Ramakrishnan, Waltham, MA (US); Nicholas Woodall, Waltham, MA (US); Andrew Wollacott, Waltham, MA (US); Yizhou Yin, Waltham, MA (US); Gregory Babcock, Waltham, MA (US); Yoshimi Kuroiwa, Tokyo (JP); Koji Ohmoto, Tokyo (JP)

(21) Appl. No.: 19/331,754

(22) Filed: Sep. 17, 2025

Related U.S. Application Data
(60) Provisional application No. 63/695,615, filed on Sep. 17, 2024.

(51) Int. Cl. C07K 16/28; A61K 39/00; A61K 48/00; A61P 27/02; C12N 15/86

(52) U.S. Cl. CPC C07K 16/2866; A61K 48/0033; A61K 48/005; A61P 27/02; C12N 15/86; A61K 2039/505; C07K 2317/569; C12N 2750/14143

(57) ABSTRACT

The present disclosure provides, among other things, anti-CSF1R single-domain antibodies (sdAbs) with high affinity to CSF1R (e.g., K_{D} of pM to low nM) and therapeutic uses of such sdAbs in effectively treating CSF1R mediated diseases, such as age-related macular degeneration (AMD). In some embodiments, an anti-CSF1R sdAb is fused to an Fc region.

### CROSS-REFERENCE TO RELATED APPLICATIONS

This application claims priority to U.S. Provisional Application No. 63/695,615, filed on Sep. 17, 2024. The contents of the aforesaid application are incorporated herein by reference in its entirety for all purposes.

### INCORPORATION BY REFERENCE OF SEQUENCE LISTING

The instant application contains a Sequence Listing which has been submitted electronically in XML format and is hereby incorporated by reference in its entirety. Said XML copy, created on Sep. 12, 2025, is named SVI-015US1_SL.xml and is 165,666 bytes in size.

### BACKGROUND

Colony stimulating factor 1 receptor (CSF1R) is a transmembrane protein in the receptor tyrosine kinase family that is expressed on the surface of macrophages and microglia. CSF1R exists in an autoinhibited form, but recognizes two ligands, which are the cytokines colony stimulating factor 1 (CSF1) and interleukin 34 (IL-34). Recognition of CSF1 or IL-34 by CSF1R serves a function in the differentiation of myeloid progenitor cells into macrophages, as well as in vivo homeostasis of macrophages in various tissue types. Macrophages function to recognize and destroy pathogens, as well as phagocytosis (ingestion of materials by cells known as phagocytes) of cellular debris.

### SUMMARY OF THE INVENTION

The present invention provides, among other things, anti-CSF1R single-domain antibodies (sdAbs) with high affinity to CSF1R (e.g., K_{D} of pM to low nM) and therapeutic uses of such sdAbs in effectively treating CSF1R mediated diseases, such as age-related macular degeneration (AMD). In some embodiments, a sdAb is fused to an Fc region. As described herein, the present invention is, in part, based on the identification of a new class of anti-CSF1R sdAbs that potently inhibit activation of human CSF1R by blocking the binding of CSF1 and IL34 to CSF1R or obstructing the dimerization domains (D4 and/or D5). In particular, the sdAbs of the present invention are characterized with high binding affinity to CSF1R (e.g., K_{D} less than 10 nM) and/or high inhibition activity of both CSF1 and IL-34 (e.g., IC₅₀ less than 15 nM). As a result, anti-CSF1R sdAbs of the present invention can be used at a lower dose to achieve therapeutic effect relative to the other anti-CSF1R antibodies or anti-CSF1 antibodies. Thus, the present invention provides inventive anti-CSF1R sdAbs that (i) inhibit the activation of human CSF1R; (ii) block the binding of both CSF1 and IL-34 to CSF1R or obstruct dimerization of CSF1R; (iii) inhibit macrophage proliferation; and (vi) have favorable biophysical and pharmacokinetic properties to allow for prolonged therapeutic effect. Inventive anti-CSF1R sdAbs of the present invention promise a more potent treatment of CSF1R mediated diseases and disorders such as AMD.

In one aspect, the present invention provides, among other things, a single domain antibody that binds to colony stimulating factor 1 receptor (CSF1R).

In one aspect, the present invention provides, among other things, a sdAb that binds to CSF1R, wherein the sdAb comprises a CDR1 comprising an amino acid sequence that is identical to or differs by no more than 2 amino acid residues from GGTFSYY (SEQ ID NO: 106), a CDR2 comprising an amino acid sequence that is identical to or differs by no more than 2 amino acid residues from SQYGSN (SEQ ID NO: 109), and a CDR3 comprising an amino acid sequence that is identical to or differs by no more than 2 amino acid residues from LRSGSDWRTIRDYDN (SEQ ID NO: 112).

In some embodiments, the sdAb comprises a CDR1 comprising an amino acid sequence that differs by no more than 1 amino acid residue from GGTFSYY (SEQ ID NO: 106), a CDR2 comprising an amino acid sequence that differs by no more than 1 amino acid residue from SQYGSN (SEQ ID NO: 109), and a CDR3 comprising an amino acid sequence that differs by no more than 1 amino acid residue from LRSGSDWRTIRDYDN (SEQ ID NO: 112).

In one aspect, the present invention provides, among other things, a sdAb that binds to CSF1R, wherein the sdAb comprises a CDR1 comprising an amino acid sequence that is identical to or differs by no more than 2 amino acid residues from AASGGTFSYYNMG (SEQ ID NO: 124), a CDR2 comprising an amino acid sequence that is identical to or differs by no more than 2 amino acid residues from VISQYGSNTY (SEQ ID NO: 127), and a CDR3 comprising an amino acid sequence that is identical to or differs by no more than 2 amino acid residues from ATLRSGSDWRTIRDYDN (SEQ ID NO: 130).

In some embodiments, the sdAb comprises a CDR1 comprising an amino acid sequence that differs by no more than 1 amino acid residue from AASGGTFSYYNMG (SEQ ID NO: 124), a CDR2 comprising an amino acid sequence that differs by no more than 1 amino acid residue from VISQYGSNTY (SEQ ID NO: 127), and a CDR3 comprising an amino acid sequence that differs by no more than 1 amino acid residue from ATLRSGSDWRTIRDYDN (SEQ ID NO: 130).

In one aspect, the present invention provides, among other things, a sdAb that binds to CSF1R, wherein the sdAb comprises a CDR1 comprising an amino acid sequence that is identical to or differs by no more than 2 amino acid residues from GGTFSYYN (SEQ ID NO: 115), a CDR2 comprising an amino acid sequence that is identical to or differs by no more than 2 amino acid residues from ISQYGSNT (SEQ ID NO: 118), and a CDR3 comprising an amino acid sequence that is identical to or differs by no more than 2 amino acid residues from ATLRSGSDWRTIRDYDN (SEQ ID NO: 121).

In some embodiments, the sdAb comprises a CDR1 comprising an amino acid sequence that differs by no more than 1 amino acid residue from GGTFSYYN (SEQ ID NO: 115), a CDR2 comprising an amino acid sequence that differs by no more than 1 amino acid residue from ISQYGSNT (SEQ ID NO: 118), and a CDR3 comprising an amino acid sequence that differs by no more than 1 amino acid residue from ATLRSGSDWRTIRDYDN (SEQ ID NO: 121).

In one aspect, the present invention provides, among other things, a sdAb that binds to CSF1R, wherein the sdAb comprises a CDR1 comprising an amino acid sequence that is identical to or differs by no more than 2 amino acid residues from YYNMG (SEQ ID NO: 133), a CDR2 comprising an amino acid sequence that is identical to or differs by no more than 2 amino acid residues from VISQYGSNTYYADSVKG (SEQ ID NO: 136), and a CDR3 comprising an amino acid sequence that is identical to or differs by no more than 2 amino acid residues from LRSGSDWRTIRDYDN (SEQ ID NO: 139).

In some embodiments, the sdAb comprises a CDR1 comprising an amino acid sequence that differs by no more than 1 amino acid residue from YYNMG (SEQ ID NO: 133), a CDR2 comprising an amino acid sequence that differs by no more than 1 amino acid residue from VISQYGSNTYYADSVKG (SEQ ID NO: 136), and a CDR3 comprising an amino acid sequence that differs by no more than 1 amino acid residue from LRSGSDWRTIRDYDN (SEQ ID NO: 139).

In one aspect, the present invention provides, among other things, a sdAb that binds to CSF1R, wherein the sdAb comprises a CDR1 comprising an amino acid sequence that is identical to or differs by no more than 2 amino acid residues from GDIFSFN (SEQ ID NO: 107), a CDR2 comprising an amino acid sequence that is identical to or differs by no more than 2 amino acid residues from APGGN (SEQ ID NO: 110), and a CDR3 comprising an amino acid sequence that is identical to or differs by no more than 2 amino acid residues from WPFSRDS (SEQ ID NO: 113).

In some embodiments, the sdAb comprises a CDR1 comprising an amino acid sequence that differs by no more than 1 amino acid residue from GDIFSFN (SEQ ID NO: 107), a CDR2 comprising an amino acid sequence that differs by no more than 1 amino acid residue from APGGN (SEQ ID NO: 110), and a CDR3 comprising an amino acid sequence that differs by no more than 1 amino acid residue from WPFSRDS (SEQ ID NO: 113).

In one aspect, the present invention provides, among other things, a sdAb that binds to CSF1R, wherein the sdAb comprises a CDR1 comprising an amino acid sequence that is identical to or differs by no more than 2 amino acid residues from AASGDIFSFNVMG (SEQ ID NO: 125), a CDR2 comprising an amino acid sequence that is identical to or differs by no more than 2 amino acid residues from TIAPGGNTN (SEQ ID NO: 156), and a CDR3 comprising an amino acid sequence that is identical to or differs by no more than 2 amino acid residues from NRWPFSRDS (SEQ ID NO: 131).

In some embodiments, the sdAb comprises a CDR1 comprising an amino acid sequence that differs by no more than 1 amino acid residue from AASGDIFSFNVMG (SEQ ID NO: 125), a CDR2 comprising an amino acid sequence that differs by no more than 1 amino acid residue from TIAPGGNTN (SEQ ID NO: 156), and a CDR3 comprising an amino acid sequence that differs by no more than 1 amino acid residue from NRWPFSRDS (SEQ ID NO: 131).

In one aspect, the present invention provides, among other things, a sdAb that binds to CSF1R, wherein the sdAb comprises a CDR1 comprising an amino acid sequence that is identical to or differs by no more than 2 amino acid residues from AASGDIFSFNVMG (SEQ ID NO: 125), a CDR2 comprising an amino acid sequence that is identical to or differs by no more than 2 amino acid residues from VIAPGGNTY (SEQ ID NO: 128), and a CDR3 comprising an amino acid sequence that is identical to or differs by no more than 2 amino acid residues from NRWPFSRDS (SEQ ID NO: 131).

In some embodiments, the sdAb comprises a CDR1 comprising an amino acid sequence that differs by no more than 1 amino acid residue from AASGDIFSFNVMG (SEQ ID NO: 125), a CDR2 comprising an amino acid sequence that differs by no more than 1 amino acid residue from VIAPGGNTY (SEQ ID NO: 128), and a CDR3 comprising an amino acid sequence that differs by no more than 1 amino acid residue from NRWPFSRDS (SEQ ID NO: 131).

In one aspect, the present invention provides, among other things, a sdAb that binds to CSF1R, wherein the sdAb comprises a CDR1 comprising an amino acid sequence that is identical to or differs by no more than 2 amino acid residues from GDIFSFNV (SEQ ID NO: 116), a CDR2 comprising an amino acid sequence that is identical to or differs by no more than 2 amino acid residues from IAPGGNT (SEQ ID NO: 119), and a CDR3 comprising an amino acid sequence that is identical to or differs by no more than 2 amino acid residues from NRWPFSRDS (SEQ ID NO: 122).

In some embodiments, the sdAb comprises a CDR1 comprising an amino acid sequence that differs by no more than 1 amino acid residue from GDIFSFNV (SEQ ID NO: 116), a CDR2 comprising an amino acid sequence that differs by no more than 1 amino acid residue from IAPGGNT (SEQ ID NO: 119), and a CDR3 comprising an amino acid sequence that differs by no more than 1 amino acid residue from NRWPFSRDS (SEQ ID NO: 122).

In one aspect, the present invention provides, among other things, a sdAb that binds to CSF1R, wherein the sdAb comprises a CDR1 comprising an amino acid sequence that is identical to or differs by no more than 2 amino acid residues from FNVMG (SEQ ID NO: 134), a CDR2 comprising an amino acid sequence that is identical to or differs by no more than 2 amino acid residues from VIAPGGNTYYADSVKG (SEQ ID NO: 137), and a CDR3 comprising an amino acid sequence that is identical to or differs by no more than 2 amino acid residues from WPFSRDS (SEQ ID NO: 140).

In some embodiments, the sdAb comprises a CDR1 comprising an amino acid sequence that differs by no more than 1 amino acid residue from FNVMG (SEQ ID NO: 134), a CDR2 comprising an amino acid sequence that differs by no more than 1 amino acid residue from VIAPGGNTYYADSVKG (SEQ ID NO: 137), and a CDR3 comprising an amino acid sequence that differs by no more than 1 amino acid residue from WPFSRDS (SEQ ID NO: 140).

In one aspect, the present invention provides, among other things, a sdAb that binds to CSF1R, wherein the sdAb comprises a CDR1 comprising an amino acid sequence that is identical to or differs by no more than 2 amino acid residues from GMAFGSS (SEQ ID NO: 108), a CDR2 comprising an amino acid sequence that is identical to or differs by no more than 2 amino acid residues from TSAGY (SEQ ID NO: 111), and a CDR3 comprising an amino acid sequence that is identical to or differs by no more than 2 amino acid residues from GPFHSTAYST (SEQ ID NO: 114).

In some embodiments, the sdAb comprises a CDR1 comprising an amino acid sequence that differs by no more than 1 amino acid residue from GMAFGSS (SEQ ID NO: 108), a CDR2 comprising an amino acid sequence that differs by no more than 1 amino acid residue from TSAGY (SEQ ID NO: 111), and a CDR3 comprising an amino acid sequence that differs by no more than 1 amino acid residue from GPFHSTAYST (SEQ ID NO: 114).

In one aspect, the present invention provides, among other things, a sdAb that binds to CSF1R, wherein the sdAb comprises a CDR1 comprising an amino acid sequence that is identical to or differs by no more than 2 amino acid residues from VASGMAFGSSPMN (SEQ ID NO: 126), a CDR2 comprising an amino acid sequence that is identical to or differs by no more than 2 amino acid residues from GVTSAGYAY (SEQ ID NO: 129), and a CDR3 comprising an amino acid sequence that is identical to or differs by no more than 2 amino acid residues from ARGPFHSTAYST (SEQ ID NO: 132).

In some embodiments, the sdAb comprises a CDR1 comprising an amino acid sequence that differs by no more than 1 amino acid residue from VASGMAFGSSPMN (SEQ ID NO: 126), a CDR2 comprising an amino acid sequence that differs by no more than 1 amino acid residue from GVTSAGYAY (SEQ ID NO: 129), and a CDR3 comprising an amino acid sequence that differs by no more than 1 amino acid residue from ARGPFHSTAYST (SEQ ID NO: 132).

In one aspect, the present invention provides, among other things, a sdAb that binds to CSF1R, wherein the sdAb comprises a CDR1 comprising an amino acid sequence that is identical to or differs by no more than 2 amino acid residues from GMAFGSSP (SEQ ID NO: 117), a CDR2 comprising an amino acid sequence that is identical to or differs by no more than 2 amino acid residues from VTSAGYA (SEQ ID NO: 120), and a CDR3 comprising an amino acid sequence that is identical to or differs by no more than 2 amino acid residues from ARGPFHSTAYST (SEQ ID NO: 123).

In some embodiments, the sdAb comprises a CDR1 comprising an amino acid sequence that differs by no more than 1 amino acid residue from GMAFGSSP (SEQ ID NO: 117), a CDR2 comprising an amino acid sequence that differs by no more than 1 amino acid residue from VTSAGYA (SEQ ID NO: 120), and a CDR3 comprising an amino acid sequence that differs by no more than 1 amino acid residue from ARGPFHSTAYST (SEQ ID NO: 123).

In one aspect, the present invention provides, among other things, a sdAb that binds to CSF1R, wherein the sdAb comprises a CDR1 comprising an amino acid sequence that is identical to or differs by no more than 2 amino acid residues from SSPMN (SEQ ID NO: 135), a CDR2 comprising an amino acid sequence that is identical to or differs by no more than 2 amino acid residues from GVTSAGYAYYADWVKG (SEQ ID NO: 138), and a CDR3 comprising an amino acid sequence that is identical to or differs by no more than 2 amino acid residues from GPFHSTAYST (SEQ ID NO: 141).

In some embodiments, the sdAb comprises a CDR1 comprising an amino acid sequence that differs by no more than 1 amino acid residue from SSPMN (SEQ ID NO: 135), a CDR2 comprising an amino acid sequence that differs by no more than 1 amino acid residue from GVTSAGYAYYADWVKG (SEQ ID NO: 138), and a CDR3 comprising an amino acid sequence that differs by no more than 1 amino acid residue from GPFHSTAYST (SEQ ID NO: 141).

In one aspect, the present invention provides, among other things, a sdAb that binds to CSF1R, wherein the sdAb comprises a CDR1 comprising an amino acid sequence of GGTFSYY (SEQ ID NO: 106), a CDR2 comprising an amino acid sequence of SQYGSN (SEQ ID NO: 109), and a CDR3 comprising an amino acid sequence of LRSGSDWRTIRDYDN (SEQ ID NO: 112).

In one aspect, the present invention provides, among other things, a sdAb that binds to CSF1R, wherein the sdAb comprises a CDR1 comprising an amino acid sequence of AASGGTFSYYNMG (SEQ ID NO: 124), a CDR2 comprising an amino acid sequence of VISQYGSNTY (SEQ ID NO: 127), and a CDR3 comprising an amino acid sequence of ATLRSGSDWRTIRDYDN (SEQ ID NO: 130).

In one aspect, the present invention provides, among other things, a sdAb that binds to CSF1R, wherein the sdAb comprises a CDR1 comprising an amino acid sequence of GGTFSYYN (SEQ ID NO: 115), a CDR2 comprising an amino acid sequence of ISQYGSNT (SEQ ID NO: 118), and a CDR3 comprising an amino acid sequence of ATLRSGSDWRTIRDYDN (SEQ ID NO: 121).

In one aspect, the present invention provides, among other things, a sdAb that binds to CSF1R, wherein the sdAb comprises a CDR1 comprising an amino acid sequence of YYNMG (SEQ ID NO: 133), a CDR2 comprising an amino acid sequence of VISQYGSNTYYADSVKG (SEQ ID NO: 136), and a CDR3 comprising an amino acid sequence of LRSGSDWRTIRDYDN (SEQ ID NO: 139).

In one aspect, the present invention provides, among other things, a sdAb that binds to CSF1R, wherein the sdAb comprises a CDR1 comprising an amino acid sequence of GDIFSFN (SEQ ID NO: 107), a CDR2 comprising an amino acid sequence of APGGN (SEQ ID NO: 110), and a CDR3 comprising an amino acid sequence of WPFSRDS (SEQ ID NO: 113).

In one aspect, the present invention provides, among other things, a sdAb that binds to CSF1R, wherein the sdAb comprises a CDR1 comprising an amino acid sequence of AASGDIFSFNVMG (SEQ ID NO: 125), a CDR2 comprising an amino acid sequence of TIAPGGNTN (SEQ ID NO: 156), and a CDR3 comprising an amino acid sequence of NRWPFSRDS (SEQ ID NO: 131).

In one aspect, the present invention provides, among other things, a sdAb that binds to CSF1R, wherein the sdAb comprises a CDR1 comprising an amino acid sequence of AASGDIFSFNVMG (SEQ ID NO: 125), a CDR2 comprising an amino acid sequence of VIAPGGNTY (SEQ ID NO: 128), and a CDR3 comprising an amino acid sequence of NRWPFSRDS (SEQ ID NO: 131).

In one aspect, the present invention provides, among other things, a sdAb that binds to CSF1R, wherein the sdAb comprises a CDR1 comprising an amino acid sequence of GDIFSFNV (SEQ ID NO: 116), a CDR2 comprising an amino acid sequence of IAPGGNT (SEQ ID NO: 119), and a CDR3 comprising an amino acid sequence of NRWPFSRDS (SEQ ID NO: 122).

In one aspect, the present invention provides, among other things, a sdAb that binds to CSF1R, wherein the sdAb comprises a CDR1 comprising an amino acid sequence of FNVMG (SEQ ID NO: 134), a CDR2 comprising an amino acid sequence of VIAPGGNTYYADSVKG (SEQ ID NO: 137), and a CDR3 comprising an amino acid sequence of WPFSRDS (SEQ ID NO: 140).

In one aspect, the present invention provides, among other things, a sdAb that binds to CSF1R, wherein the sdAb comprises a CDR1 comprising an amino acid sequence of GMAFGSS (SEQ ID NO: 108), a CDR2 comprising an amino acid sequence of TSAGY (SEQ ID NO: 111), and a CDR3 comprising an amino acid sequence of GPFHSTAYST (SEQ ID NO: 114).

In one aspect, the present invention provides, among other things, a sdAb that binds to CSF1R, wherein the sdAb comprises a CDR1 comprising an amino acid sequence of VASGMAFGSSPMN (SEQ ID NO: 126), a CDR2 comprising an amino acid sequence of GVTSAGYAY (SEQ ID NO: 129), and a CDR3 comprising an amino acid sequence of ARGPFHSTAYST (SEQ ID NO: 132).

In one aspect, the present invention provides, among other things, a sdAb that binds to CSF1R, wherein the sdAb comprises a CDR1 comprising an amino acid sequence of GMAFGSSP (SEQ ID NO: 117), a CDR2 comprising an amino acid sequence of VTSAGYA (SEQ ID NO: 120), and a CDR3 comprising an amino acid sequence of ARGPFHSTAYST (SEQ ID NO: 123).

In one aspect, the present invention provides, among other things, a sdAb that binds to CSF1R, wherein the sdAb comprises a CDR1 comprising an amino acid sequence of SSPMN (SEQ ID NO: 135), a CDR2 comprising an amino acid sequence of GVTSAGYAYYADWVKG (SEQ ID NO: 138), and a CDR3 comprising an amino acid sequence of GPFHSTAYST (SEQ ID NO: 141).

In some embodiments, the sdAb comprises an amino acid sequence at least 85%, 90%, 95%, 96%, 97%, 98%, or 99% identical to any one of SEQ ID NOs: 1-3 or 101-105. In some embodiments, the sdAb comprises an amino acid sequence identical to any one of SEQ ID NOs: 1-3 or 101-105.

In some embodiments, the sdAb comprises an amino acid sequence at least 85%, 90%, 95%, 96%, 97%, 98%, or 99% identical to SEQ ID NO: 1. In some embodiments, the sdAb comprises an amino acid sequence at least 85% identical to SEQ ID NO: 1. In some embodiments, the sdAb comprises an amino acid sequence at least 90% identical to SEQ ID NO: 1. In some embodiments, the sdAb comprises an amino acid sequence at least 95% identical to SEQ ID NO: 1. In some embodiments, the sdAb comprises an amino acid sequence at least 96% identical to SEQ ID NO: 1. In some embodiments, the sdAb comprises an amino acid sequence 97% identical to SEQ ID NO: 1. In some embodiments, the sdAb comprises an amino acid sequence at least 98% identical to SEQ ID NO: 1. In some embodiments, the sdAb comprises an amino acid sequence at least 99% identical to SEQ ID NO: 1. In some embodiments, the sdAb comprises an amino acid sequence identical to SEQ ID NO: 1. In some embodiments, the sdAb comprises an amino acid sequence of SEQ ID NO: 1.

In some embodiments, the sdAb comprises an amino acid sequence at least 85%, 90%, 95%, 96%, 97%, 98%, or 99% identical to SEQ ID NO: 2. In some embodiments, the sdAb comprises an amino acid sequence at least 85% identical to SEQ ID NO: 2. In some embodiments, the sdAb comprises an amino acid sequence at least 90% identical to SEQ ID NO: 2. In some embodiments, the sdAb comprises an amino acid sequence at least 95% identical to SEQ ID NO: 2. In some embodiments, the sdAb comprises an amino acid sequence at least 96% identical to SEQ ID NO: 2. In some embodiments, the sdAb comprises an amino acid sequence at least 97% identical to SEQ ID NO: 2. In some embodiments, the sdAb comprises an amino acid sequence at least 98% identical to SEQ ID NO: 2. In some embodiments, the sdAb comprises an amino acid sequence at least 99% identical to SEQ ID NO: 2. In some embodiments, the sdAb comprises an amino acid sequence identical to SEQ ID NO: 2. In some embodiments, the sdAb comprises an amino acid sequence of SEQ ID NO: 2.

In some embodiments, the sdAb comprises an amino acid sequence at least 85%, 90%, 95%, 96%, 97%, 98%, or 99% identical to SEQ ID NO: 3. In some embodiments, the sdAb comprises an amino acid sequence at least 85% identical to SEQ ID NO: 3. In some embodiments, the sdAb comprises an amino acid sequence at least 90% identical to SEQ ID NO: 3. In some embodiments, the sdAb comprises an amino acid sequence at least 95% identical to SEQ ID NO: 3. In some embodiments, the sdAb comprises an amino acid sequence at least 96% identical to SEQ ID NO: 3. In some embodiments, the sdAb comprises an amino acid sequence at least 97% identical to SEQ ID NO: 3. In some embodiments, the sdAb comprises an amino acid sequence at least 98% identical to SEQ ID NO: 3. In some embodiments, the sdAb comprises an amino acid sequence at least 99% identical to SEQ ID NO: 3. In some embodiments, the sdAb comprises an amino acid sequence identical to SEQ ID NO: 3. In some embodiments, the sdAb comprises an amino acid sequence of SEQ ID NO: 3.

In some embodiments, the sdAb comprises an amino acid sequence at least 85%, 90%, 95%, 96%, 97%, 98%, or 99% identical to SEQ ID NO: 101. In some embodiments, the sdAb comprises an amino acid sequence at least 85% identical to SEQ ID NO: 101. In some embodiments, the sdAb comprises an amino acid sequence at least 90% identical to SEQ ID NO: 101. In some embodiments, the sdAb comprises an amino acid sequence at least 95% identical to SEQ ID NO: 101. In some embodiments, the sdAb comprises an amino acid sequence at least 96% identical to SEQ ID NO: 101. In some embodiments, the sdAb comprises an amino acid sequence at least 97% identical to SEQ ID NO: 101. In some embodiments, the sdAb comprises an amino acid sequence at least 98% identical to SEQ ID NO: 101. In some embodiments, the sdAb comprises an amino acid sequence at least 99% identical to SEQ ID NO: 101. In some embodiments, the sdAb comprises an amino acid sequence identical to SEQ ID NO: 101. In some embodiments, the sdAb comprises an amino acid sequence of SEQ ID NO: 101.

In some embodiments, the sdAb comprises an amino acid sequence at least 85%, 90%, 95%, 96%, 97%, 98%, or 99% identical to SEQ ID NO: 102. In some embodiments, the sdAb comprises an amino acid sequence at least 85% identical to SEQ ID NO: 102. In some embodiments, the sdAb comprises an amino acid sequence at least 90% identical to SEQ ID NO: 102. In some embodiments, the sdAb comprises an amino acid sequence at least 95% identical to SEQ ID NO: 102. In some embodiments, the sdAb comprises an amino acid sequence at least 96% identical to SEQ ID NO: 102. In some embodiments, the sdAb comprises an amino acid sequence at least 97% identical to SEQ ID NO: 102. In some embodiments, the sdAb comprises an amino acid sequence at least 98% identical to SEQ ID NO: 102. In some embodiments, the sdAb comprises an amino acid sequence at least 99% identical to SEQ ID NO: 102. In some embodiments, the sdAb comprises an amino acid sequence identical to SEQ ID NO: 102. In some embodiments, the sdAb comprises an amino acid sequence of SEQ ID NO: 102.

In some embodiments, the sdAb comprises an amino acid sequence at least 85%, 90%, 95%, 96%, 97%, 98%, or 99% identical to SEQ ID NO: 103. In some embodiments, the sdAb comprises an amino acid sequence at least 85% identical to SEQ ID NO: 103. In some embodiments, the sdAb comprises an amino acid sequence at least 90% identical to SEQ ID NO: 103. In some embodiments, the sdAb comprises an amino acid sequence at least 95% identical to SEQ ID NO: 103. In some embodiments, the sdAb comprises an amino acid sequence at least 96% identical to SEQ ID NO: 103. In some embodiments, the sdAb comprises an amino acid sequence at least 97% identical to SEQ ID NO: 103. In some embodiments, the sdAb comprises an amino acid sequence at least 98% identical to SEQ ID NO: 103. In some embodiments, the sdAb comprises an amino acid sequence at least 99% identical to SEQ ID NO: 103. In some embodiments, the sdAb comprises an amino acid sequence identical to SEQ ID NO: 103. In some embodiments, the sdAb comprises an amino acid sequence of SEQ ID NO: 103.

In some embodiments, the sdAb comprises an amino acid sequence at least 85%, 90%, 95%, 96%, 97%, 98%, or 99% identical to SEQ ID NO: 104. In some embodiments, the sdAb comprises an amino acid sequence at least 85% identical to SEQ ID NO: 104. In some embodiments, the sdAb comprises an amino acid sequence at least 90% identical to SEQ ID NO: 104. In some embodiments, the sdAb comprises an amino acid sequence at least 95% identical to SEQ ID NO: 104. In some embodiments, the sdAb comprises an amino acid sequence at least 96% identical to SEQ ID NO: 104. In some embodiments, the sdAb comprises an amino acid sequence at least 97% identical to SEQ ID NO: 104. In some embodiments, the sdAb comprises an amino acid sequence at least 98% identical to SEQ ID NO: 104. In some embodiments, the sdAb comprises an amino acid sequence at least 99% identical to SEQ ID NO: 104. In some embodiments, the sdAb comprises an amino acid sequence identical to SEQ ID NO: 104. In some embodiments, the sdAb comprises an amino acid sequence of SEQ ID NO: 104.

In some embodiments, the sdAb comprises an amino acid sequence at least 85%, 90%, 95%, 96%, 97%, 98%, or 99% identical to SEQ ID NO: 105. In some embodiments, the sdAb comprises an amino acid sequence at least 85% identical to SEQ ID NO: 105. In some embodiments, the sdAb comprises an amino acid sequence at least 90% identical to SEQ ID NO: 105. In some embodiments, the sdAb comprises an amino acid sequence at least 95% identical to SEQ ID NO: 105. In some embodiments, the sdAb comprises an amino acid sequence at least 96% identical to SEQ ID NO: 105. In some embodiments, the sdAb comprises an amino acid sequence at least 97% identical to SEQ ID NO: 105. In some embodiments, the sdAb comprises an amino acid sequence at least 98% identical to SEQ ID NO: 105. In some embodiments, the sdAb comprises an amino acid sequence at least 99% identical to SEQ ID NO: 105. In some embodiments, the sdAb comprises an amino acid sequence identical to SEQ ID NO: 105. In some embodiments, the sdAb comprises an amino acid sequence of SEQ ID NO: 105.

In one aspect, the present invention provides, among other things, a sdAb that binds to D5 of CSF1R, corresponding to residues 402-502 of SEQ ID NO: 142. In some embodiments, the sdAb binds to amino acid sequence of SEQ ID NO: 158. In some embodiments, the sdAb binds to contiguous residues of D5. In some embodiments, the sdAb binds to non-contiguous residues of D5.

In some embodiments, the sdAb binds to human CSF1R. In some embodiments, the sdAb binds to cynomolgus monkey CSF1R. In some embodiments, the sdAb binds to human CSF1R and cynomolgus monkey CSF1R.

In some embodiments, the sdAb does not bind to mouse CSF1R. In some embodiments, the sdAb binds to mouse CSF1R with a humanized D5 domain. In some embodiments, the sdAb does not bind to mouse CSF1R, but does bind to human CSF1R and mouse CSF1R with a humanized D5 domain. In some embodiments, the sdAb does not bind to mouse CSF1R, but does bind to human CSF1R, cynomolgus monkey CSF1R, and mouse CSF1R with a humanized D5 domain. In some embodiments, the sdAb does not bind to a polypeptide consisting of the amino acid sequence of SEQ ID NO: 160, but does bind to a polypeptide consisting of the amino acid sequence of SEQ ID NO: 161.

In some embodiments, the sdAb binds to one or more amino acid residues selected from I407, A420, W496, and A497 of human CSF1R (SEQ ID NO: 142), or a combination thereof. In some embodiments, the sdAb binds to at least two residues selected from I407, A420, W496, and A497 of human CSF1R (SEQ ID NO: 142). In some embodiments, the sdAb binds to amino acid residue W496 of human CSF1R (SEQ ID NO: 142). In some embodiments, the sdAb binds to amino acid residue A497 of human CSF1R (SEQ ID NO: 142). In some embodiments, the sdAb binds to amino acid residue A420 of human CSF1R (SEQ ID NO: 142). In some embodiments, the sdAb binds to amino acid residue I407 of human CSF1R (SEQ ID NO: 142).

In some embodiments, the sdAb binds to amino acid residues W496 and A497 of human CSF1R (SEQ ID NO: 142). In some embodiments, the sdAb binds to amino acid residues A420 and I407 of human CSF1R (SEQ ID NO: 142).

In some embodiments, the sdAb binds to CSF1R with a binding affinity (K_{D}) between 1 and 12 nM. In some embodiments, the sdAb binds to CSF1R with a binding affinity (K_{D}) between 1 and 11 nM. In some embodiments, the sdAb binds to CSF1R with a binding affinity (K_{D}) between 1 and 10 nM. In some embodiments, the sdAb binds to CSF1R with a binding affinity (K_{D}) between 1 and 9 nM. In some embodiments, the sdAb binds to CSF1R with a binding affinity (K_{D}) between 1 and 8 nM. In some embodiments, the sdAb binds to CSF1R with a binding affinity (K_{D}) between 1 and 7 nM. In some embodiments, the sdAb binds to CSF1R with a binding affinity (K_{D}) between 1 and 6 nM. In some embodiments, the sdAb binds to CSF1R with a binding affinity (K_{D}) between 1 and 5 nM. In some embodiments, the sdAb binds to CSF1R with a binding affinity (K_{D}) between 1 and 4 nM. In some embodiments, the sdAb binds to CSF1R with a binding affinity (K_{D}) between 1 and 3 nM. In some embodiments, the sdAb binds to CSF1R with a binding affinity (K_{D}) between 1 and 2 nM.

In some embodiments, the sdAb binds to cynomolgus CSF1R with a binding affinity (K_{D}) of between 2 and 6 nM. In some embodiments, the sdAb binds to cynomolgus CSF1R with a binding affinity (K_{D}) of between 2 and 5 nM. In some embodiments, the sdAb binds to cynomolgus CSF1R with a binding affinity (K_{D}) of between 2 and 4 nM. In some embodiments, the sdAb binds to cynomolgus CSF1R with a binding affinity (K_{D}) of between 2 and 3 nM. In some embodiments, the K_{D} value is determined by the binding described in Example 1.

In some embodiments, the sdAb inhibits CSF1 activity with an IC₅₀ value between 0.5 and 4 nM. In some embodiments, the sdAb inhibits CSF1 activity with an IC₅₀ value between 0.5 and 3 nM. In some embodiments, the sdAb inhibits CSF1 activity with an IC₅₀ value between 0.5 and 2 nM. In some embodiments, the sdAb inhibits CSF1 activity with an IC₅₀ value between 0.5 and 1 nM.

In some embodiments, the sdAb inhibits IL-34 activity with an IC₅₀ value between 2 and 22 nM. In some embodiments, the sdAb inhibits IL-34 activity with an IC₅₀ value between 2 and 20 nM. In some embodiments, the sdAb inhibits IL-34 activity with an IC₅₀ value between 2 and 18 nM. In some embodiments, the sdAb inhibits IL-34 activity with an IC₅₀ value between 2 and 16 nM. In some embodiments, the sdAb inhibits IL-34 activity with an IC₅₀ value between 2 and 14 nM. In some embodiments, the sdAb inhibits IL-34 activity with an IC₅₀ value between 2 and 12 nM. In some embodiments, the sdAb inhibits IL-34 activity with an IC₅₀ value between 2 and 10 nM. In some embodiments, the sdAb inhibits IL-34 activity with an IC₅₀ value between 2 and 9 nM. In some embodiments, the sdAb inhibits IL-34 activity with an IC₅₀ value between 2 and 8 nM. In some embodiments, the sdAb inhibits IL-34 activity with an IC₅₀ value between 2 and 7 nM. In some embodiments, the sdAb inhibits IL-34 activity with an IC₅₀ value between 2 and 6 nM. In some embodiments, the sdAb inhibits IL-34 activity with an IC₅₀ value between 2 and 5 nM. In some embodiments, the sdAb inhibits IL-34 activity with an IC₅₀ value between 2 and 4 nM. In some embodiments, the sdAb inhibits IL-34 activity with an IC₅₀ value between 2 and 3 nM.

In some embodiments, the IC₅₀ value is determined by cellular reporter assay and macrophage proliferation assay. In some embodiments, the IC₅₀ value is determined by cellular reporter assay or macrophage proliferation assay. In some embodiments, the IC₅₀ value is determined by cellular reporter assay. In some embodiments, the IC₅₀ value is determined by macrophage proliferation assay. In some embodiments, the IC₅₀ value is determined by the cellular reporter assay and/or the macrophage proliferation assay described in Example 2.

In some embodiments, the sdAb is a llama VHH. In some embodiments, the sdAb is a humanized VHH.

In some embodiments, the sdAb is fused to an Fc region. In some embodiments, the Fc region is derived from IgG1, IgG2, IgG3, or IgG4. In some embodiments, the Fc region is derived from IgG1. In some embodiments, the Fc region is derived from IgG2. In some embodiments, the Fc region is derived from IgG3. In some embodiments, the Fc region is derived from IgG4. In some embodiments, the Fc region is modified.

In some embodiments, a nucleic acid encodes the sdAb described herein.

In some embodiments, a vector comprises the nucleic acid described herein. In some embodiments, the vector is a viral vector. In some embodiments, the vector is an adeno-associated virus (AAV) vector.

In some embodiments, the AAV vector is derived from AAV1, AAV2, AAV3, AAV4, AAV5, AAV6, AAV7, AAV8, AAV9, AAV10, AAV10, AAV11, AAV12, AAV13. In some embodiments, the AAV vector is derived from AAV1. In some embodiments, the AAV vector is derived from AAV2. In some embodiments, the AAV vector is derived from AAV3. In some embodiments, the AAV vector is derived from AAV4. In some embodiments, the AAV vector is derived from AAV5. In some embodiments, the AAV vector is derived from AAV6. In some embodiments, the AAV vector is derived from AAV7. In some embodiments, the AAV vector is derived from AAV8. In some embodiments, the AAV vector is derived from AAV9. In some embodiments, the AAV vector is derived from AAV10. In some embodiments, the AAV vector is derived from AAV11. In some embodiments, the AAV vector is derived from AAV12. In some embodiments, the AAV vector is derived from AAV13.

In one aspect, the present invention provides, among other things, an AAV vector comprising a nucleic acid encoding a sdAb that binds to CSF1R.

In some embodiments, a liquid nanoparticle (LNP) comprises the nucleic acid described herein. In some embodiments, the LNP comprises the vector described herein. In some embodiments, the LNP comprises the AAV vector described herein.

In some embodiments, a host cell comprises the nucleic acid described herein. In some embodiments, the host cell comprises the vector described herein. In some embodiments, the host cell comprises the AAV vector described herein.

In some embodiments, the present invention provides, among other things, a method of producing a sdAb described herein. In some embodiments, the method comprises culturing the host cell described herein under conditions that allow production of the sdAb. In some embodiments, the method further comprises isolating the sdAb.

In some embodiments, a pharmaceutical composition comprises the sdAb described herein and one or more physiologically acceptable carriers, excipients, or diluents. In some embodiments, a pharmaceutical composition comprises the nucleic acid described herein and one or more physiologically acceptable carriers, excipients, or diluents. In some embodiments, a pharmaceutical composition comprises the vector described herein and one or more physiologically acceptable carriers, excipients, or diluents. In some embodiments, a pharmaceutical composition comprises the AAV vector described herein and one or more physiologically acceptable carriers, excipients, or diluents. In some embodiments, a pharmaceutical composition comprises the LNP described herein and one or more physiologically acceptable carriers, excipients, or diluents. In some embodiments, a pharmaceutical composition comprises the host cell described herein and one or more physiologically acceptable carriers, excipients, or diluents.

In one aspect, the present invention provides, among other things, a method of treating a disease or disorder associated with CSF1R in a subject in need thereof. In some embodiments, the method comprises administering to the subject the sdAb described herein. In some embodiments, the method comprises administering to the subject the nucleic acid described herein. In some embodiments, the method comprises administering to the subject the vector described herein. In some embodiments, the method comprises administering to the subject the AAV vector described herein. In some embodiments, the method comprises administering to the subject the LNP described herein. In some embodiments, the method comprises administering to the subject the host cell described herein. In some embodiments, the method comprises administering to the subject the pharmaceutical composition described herein.

In one aspect, the present invention provides, among other things, a method of treating an ocular disease or disorder associated with CSF1R in a subject in need thereof. In some embodiments, the method comprises treating Age-related macular degeneration (AMD), Stargardt disease, Retinitis pigmentosa, Central retinal vein occlusion (CRVO), Choroidal neovascularization (CNV), Diabetic retinopathy, Proliferative diabetic retinopathy (PDR), Proliferative vitreoretinopathy, Retinal edema, Retinal vein occlusion (RVO), Retinopathy of prematurity (ROP), Sequela associated with retinal ischemia, Anterior segment neovascularization, Corneal neovascularization, Posterior segment neovascularization, Polypoidal choroidal vasculopathy, Diabetic macular edema (DME), Noninfectious uveitis (NIU), Uveitis, Dry eye syndrome, or Glaucoma. In some embodiments, the method comprises administering to the subject the sdAb described herein. In some embodiments, the method comprises administering to the subject the nucleic acid described herein. In some embodiments, the method comprises administering to the subject the vector described herein. In some embodiments, the method comprises administering to the subject the AAV vector described herein. In some embodiments, the method comprises administering to the subject the LNP described herein. In some embodiments, the method comprises administering to the subject the host cell described herein. In some embodiments, the method comprises administering to the subject the pharmaceutical composition described herein.

In one aspect, the present invention provides, among other things, a method of treating age-related macular degeneration (AMD) in a subject in need thereof. In some embodiments, the method comprises treating dry AMD. In some embodiments, the method comprises treating wet AMD. In some embodiments, the method comprises administering to the subject the sdAb described herein. In some embodiments, the method comprises administering to the subject the nucleic acid described herein. In some embodiments, the method comprises administering to the subject the vector described herein. In some embodiments, the method comprises administering to the subject the AAV vector described herein. In some embodiments, the method comprises administering to the subject the LNP described herein. In some embodiments, the method comprises administering to the subject the host cell described herein. In some embodiments, the method comprises administering to the subject the pharmaceutical composition described herein.

In one aspect, the present invention provides, among other things, a method of treating dry AMD in subject in need thereof, wherein the method comprises administering a CSF1R inhibitor to the subject. In some embodiments, the CSF1R inhibitor is an anti-CSF1R antibody. In some embodiments, the CSF1R inhibitor is an anti-CSF1R sdAb described herein.

In some embodiments, the sdAb described herein is used for treating a disease or disorder associated with CSF1R. In some embodiments, the nucleic acid described herein is used for treating a disease or disorder associated with CSF1R. In some embodiments, the vector described herein is used for treating a disease or disorder associated with CSF1R. In some embodiments, the AAV vector described herein is used for treating a disease or disorder associated with CSF1R. In some embodiments, the LNP described herein is used for treating a disease or disorder associated with CSF1R. In some embodiments, the host cell described herein is used for treating a disease or disorder associated with CSF1R. In some embodiments, the pharmaceutical composition described herein is used for treating a disease or disorder associated with CSF1R.

In some embodiments, the sdAb described herein is used for treating AMD. In some embodiments, the nucleic acid described herein is used for treating AMD. In some embodiments, the vector described herein is used for treating AMD. In some embodiments, the AAV vector described herein is used for treating AMD. In some embodiments, the LNP described herein is used for treating AMD. In some embodiments, the host cell described herein is used for AMD. In some embodiments, the pharmaceutical composition described herein is used for treating AMD. In some embodiments, the AMD is dry AMD. In some embodiments, the AMD is wet AMD.

In some embodiments, the sdAb described herein is used in the manufacture of a medicament for treating a disease or disorder associated with CSF1R. In some embodiments, the nucleic acid described herein is used in the manufacture of a medicament for treating a disease or disorder associated with CSF1R. In some embodiments, the vector described herein is used in the manufacture of a medicament for treating a disease or disorder associated with CSF1R. In some embodiments, the AAV vector described herein is used in the manufacture of a medicament for treating a disease or disorder associated with CSF1R. In some embodiments, the LNP described herein is used in the manufacture of a medicament for treating a disease or disorder associated with CSF1R. In some embodiments, the host cell described herein is used in the manufacture of a medicament for treating a disease or disorder associated with CSF1R. In some embodiments, the pharmaceutical composition described herein is used in the manufacture of a medicament for treating a disease or disorder associated with CSF1R.

In some embodiments, the sdAb described herein is used in the manufacture of a medicament for treating AMD. In some embodiments, the nucleic acid described herein is used in the manufacture of a medicament for treating AMD. In some embodiments, the vector described herein is used in the manufacture of a medicament for treating AMD. In some embodiments, the AAV vector described herein is used in the manufacture of a medicament for treating AMD. In some embodiments, the LNP described herein is used in the manufacture of a medicament for treating AMD. In some embodiments, the host cell described herein is used in the manufacture of a medicament for AMD. In some embodiments, the pharmaceutical composition described herein is used in the manufacture of a medicament for treating AMD.

In one aspect, the present invention provides, among other things, a sdAb comprising means for binding to CSF1R. In one aspect, the present invention provides, among other things, a sdAb comprising means for binding to D5 of CSF1R, corresponding to residues 402-502 of SEQ ID NO: 142. In one aspect, the present invention provides, among other thing, a sdAb comprising means for preventing dimerization of CSF1R.

In one aspect, the present invention provides, among other things, a method of inhibiting CSF1R signaling by administering a sdAb to a cell. In some embodiments, the sdAb comprises means for binding to D5 of CSF1R, corresponding to residues 402-502 of SEQ ID NO: 142.

In one aspect, the present invention provides, among other things, a method of inhibiting macrophage proliferation by administering a sdAb to a cell. In some embodiments, the sdAb comprises means for binding to D5 of CSF1R, corresponding to residues 402-502 of SEQ ID NO: 142.

In one aspect, the present invention provides, among other things, a method of treating a disease or disorder associated with CSF1R comprising administering to a subject in need thereof a pharmaceutical composition comprising a sdAb comprising means for binding to CSF1R and one or more physiologically acceptable carrier, excipients, or diluents. In one aspect, the present invention provides, among other things, a method of treating a disease or disorder associated with CSF1R comprising administering to a subject in need thereof a pharmaceutical composition comprising a sdAb comprising means for binding to D5 of CSF1R, corresponding to residues 402-502 of SEQ ID NO: 142, and one or more physiologically acceptable carrier, excipients, or diluents.

In one aspect, the present invention provides, among other things, a method of treating AMD comprising administering to a subject in need thereof a pharmaceutical composition comprising a sdAb comprising means for binding to CSF1R and one or more physiologically acceptable carrier, excipients, or diluents. In one aspect, the present invention provides, among other things, a method of treating AMD comprising administering to a subject in need thereof a pharmaceutical composition comprising a sdAb comprising means for binding to D5 of CSF1R, corresponding to residues 402-502 of SEQ ID NO: 142, and one or more physiologically acceptable carrier, excipients, or diluents.

In one aspect, the present invention provides, among other things, a method of treating a disease or disorder associated with CSF1R comprising administering to a subject in need thereof a pharmaceutical composition comprising an AAV vector encoding a sdAb comprising means for binding to CSF1R and one or more physiologically acceptable carrier, excipients, or diluents. In one aspect, the present invention provides, among other things, a method of treating a disease or disorder associated with CSF1R comprising administering to a subject in need thereof a pharmaceutical composition comprising an AAV vector encoding a sdAb comprising means for binding to D5 of CSF1R, corresponding to residues 402-502 of SEQ ID NO: 142, and one or more physiologically acceptable carrier, excipients, or diluents.

In one aspect, the present invention provides, among other things, a method of treating AMD comprising administering to a subject in need thereof a pharmaceutical composition comprising an AAV vector encoding a sdAb comprising means for binding to CSF1R and one or more physiologically acceptable carrier, excipients, or diluents. In one aspect, the present invention provides, among other things, a method of treating AMD comprising administering to a subject in need thereof a pharmaceutical composition comprising an AAV vector encoding a sdAb comprising means for binding to D5 of CSF1R, corresponding to residues 402-502 of SEQ ID NO: 142, and one or more physiologically acceptable carrier, excipients, or diluents.

In one aspect, the present invention provides, among other things, a sdAb that binds to CSF1R comprising a CDR1 comprising a consensus sequence of GX₁TFSX₂YN (SEQ ID NO: 147), wherein X₁ comprises G, L, or F, and X₂ comprises S or Y; a CDR2 comprising an amino acid sequence of ISQYGSNT (SEQ ID NO: 150); and a CDR3 comprising a consensus sequence of TLRSGSDWRTX₃RDYDNW (SEQ ID NO: 153), wherein X₃ comprises I or M.

In one aspect, the present invention provides, among other things, a sdAb that binds to CSF1R comprising a CDR1 comprising a consensus sequence of GX₄X₅FGSX₆ (SEQ ID NO: 148), wherein X₄ comprises F, M, or L, X₅ comprises T or A, and X₆ comprises T or S; a CDR2 comprising a consensus sequence of X₇TSX₈GYX₉ (SEQ ID NO: 151), wherein X₇ comprises V or T, X₈ comprises S, A, or G, and X₉ comprises S, A, or T; and a CDR3 comprising a consensus sequence of RGPFHSX₁₀AYX₁₁TR (SEQ ID NO: 154), wherein X₁₀ comprises T or I, and X₁₁ comprises A or S.

In one aspect, the present invention provides, among other things, a sdAb that binds to CSF1R comprising a CDR1 comprising a consensus sequence of GX₂₁IFX₂₂X₂₃X₂₄V (SEQ ID NO: 149), wherein X₂₁ comprises D or N, X₂₂ comprises R or S, X₂₃ comprises F or S, and X₂₄ comprises T or N; a CDR2 comprising a consensus sequence of IX₂₅X₂₆GGX₂₇T (SEQ ID NO: 152), wherein X₂₅ comprises T, A, or S, X₂₆ comprises S, P, or T, and X₂₇ comprises R, N, or K; and a CDR3 comprising a consensus sequence of X₂₈LPFX₂₉RDS (SEQ ID NO: 155), wherein X₂₈ comprises R, T, or S, and X₂₉ comprises R or S.

### BRIEF DESCRIPTION OF DRAWINGS

The drawings herein are for illustrative purposes and should not be limiting.

FIG. 1 is exemplary schematic representations of the exemplary anti-CSF1R sdAbs and sdAb-Fc formats of the present invention.

FIGS. 2A-2B are exemplary graphs illustrating the inhibitory activity (IC₅₀) of CSF1R sdAbs in the His-tagged sdAb-only format in the presence of the CSF1 and IL-34 ligands as measured by cellular reporter assays, wherein the readout is luciferase luminescence. FIG. 2A shows the data for the Ab1 Parental, Ab1-9, Ab1-12, Ab1-18, and Ab1-20 clones. FIG. 2B shows the data for the Ab2 Parental, Ab2-10, and Ab3 Parental clones.

FIGS. 3A-3B are exemplary graphs illustrating the inhibitory activity (IC₅₀) of CSF1R sdAbs in the sdAb monovalent-Fc fusion format in the presence of the CSF1 and IL-34 ligands as measured by cellular reporter assays, wherein the readout is luciferase luminescence. FIG. 3A shows the data for the Ab1 Parental, Ab1-9, Ab1-12, Ab1-18, and Ab1-20 clones. FIG. 3B shows the data from for Ab2 Parental, Ab2-10, and Ab3 Parental clones.

FIGS. 4A-4B are exemplary graphs illustrating the inhibitory activity (IC₅₀) of CSF1R sdAbs in the His-tagged sdAb-only format as measured by macrophage proliferation assays in the presence of CSF1R ligands CSF1 and IL-34. FIG. 4A shows the data for the Ab1 Parental, Ab1-9, Ab1-12, Ab1-18, and Ab1-20 clones. FIG. 4B shows the data for the Ab2 Parental, Ab2-10, and Ab3 Parental clones.

FIG. 5 are exemplary graphs illustrating the inhibitory activity (IC₅₀) of CSF1R sdAbs in the sdAb monovalent Fc-fusion format as measured by macrophage proliferation assays in the presence of the CSF1R ligands CSF1 and IL-34.

FIG. 6 is a series of exemplary graphs illustrating epitope mapping of Ab1, Ab2, and Ab3 antibodies to a human CSF1R [h(D1D2D3D4D5)], a mouse CSF1R [m(D1D2D3D4D5)], and a mouse CSF1R with a humanized D5 domain [m(D1D2D3D4)h(D5)].

FIG. 7A is a series of exemplary graphs illustrating the key residues that describe the core epitope of the parental Ab1 and Ab2 sdAbs. FIG. 7B is an exemplary cryogenic electron microscopy (cryo-EM) model of the complex between Ab1 and CSF1R_D5 domain.

FIG. 8 is a series of exemplary graphs showing the expression levels of retinal marker genes (e.g., RPE65 and OPN1SW) in retinal pigmented epithelium and photoreceptor cells in the retina and choroid after administration of an anti-CSF1R antibody as evaluated by qPCR.

### DEFINITIONS

Antibody: As used herein, the term “antibody” refers to immunoglobulin molecules and immunologically active portions of immunoglobulin (Ig) molecules, i.e., molecules that contain an antigen binding site that binds (immunoreacts with) an antigen. By “binds” or “immunoreacts with” is meant that the antibody reacts with one or more antigenic determinants of the desired. Antibodies include antibody fragments. Antibodies also include, but are not limited to, polyclonal, monoclonal, chimeric dAb (domain antibody), single chain, Fab, Fab′, F(ab′)2 fragments, scFvs, single-domain antibodies (sdAb), and VHHs. An antibody may be a whole antibody, or immunoglobulin, or an antibody fragment.

Antibody dependent cytotoxicity: As used herein, the term “antibody-dependent cytotoxicity” or “ADCC” refers to lysis of human target cells by an antibody according to the invention in the presence of effector cells.

Fab arm exchange: The term, “Fab arm exchange” refers to the phenomenon that IgG4 antibodies can exchange ‘half-molecules’, an activity termed Fab arm exchange herein. Especially in bispecific or biparatopic molecules, this results in functionally monovalent antibodies with unknown specificity and hence, potentially, reduced therapeutic efficacy. Mutations can be introduced in the Fc region to inhibit the Fab arm exchange. It is known that S228P mutation can prevent IgG4 FAE to undetectable levels both in vitro and in vivo.

Fc region: As used herein, the term “Fc region” refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

Humanized antibody or sdAb: The term “humanized antibody or sdAb” includes non-human (e.g., murine) antibodies or sdAbs that are specific immunoglobulin chains, chimeric immunoglobulins, or fragments thereof that contain minimal non-human (e.g., murine) sequences. Typically, humanized antibodies or sdAbs are human immunoglobulins in which residues from the complementary determining region (CDR) are replaced by residues from the CDR of a non-human species (e.g., mouse, rat, rabbit, hamster) that have the desired specificity, affinity, and capability (Jones et al., Nature 321:522-525, 1986; Riechmann et al., Nature 332:323-327, 1988; Verhoeyen et al., Science 239:1534-1536, 1988).

Increased ADCC: The term “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with an acceptable, art-recognized assay.

Kᵢ or K_{D}: As used herein, the term “K_{D}”, as used herein, refers to the dissociation constant of a particular antibody-antigen interaction as is known in the art, and would apply as a parameter of the binding affinity of a targeting moiety to its cognate ligand for the subject compositions.

IC₅₀: As used herein, the term “IC₅₀” refers to the concentration needed to inhibit half of the maximum biological response of the ligand agonist, and is generally determined by competition binding assays.

EC₅₀: As used herein, the term “EC₅₀” refers to a half maximal effective concentration. The term EC₅₀ refers to the concentration of a drug, antibody or toxicant which induces a response halfway between the baseline and maximum after a specified exposure time. More simply, EC₅₀ can be defined as the concentration required to obtain a 50% of the desired effect.

CSF1R: As used herein, the term “CSF1R” refers to colony stimulating factor 1 receptor. In some embodiments, CSF1R is also known as macrophage colony stimulating factor 1 receptor.

Linker: As used herein, the term “linker” refers to a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. In some embodiments, the linker may contain amino acid residues that provide flexibility. Thus, the linker peptide may predominantly include the following amino acid residues: Gly (G), Ser (S), Ala (A), or Thr (T). The linker peptide should have a length that is adequate to link two molecules in such a way that they assume the correct conformation relative to one another so that they retain the desired activity. Suitable lengths for this purpose include at least one and not more than 30 amino acid residues. In one embodiment, the linker is from about 1 to 30 amino acids in length. In another embodiment, the linker is from about 1 to 15 amino acids in length. In addition, the amino acid residues selected for inclusion in the linker peptide should exhibit properties that do not interfere significantly with the activity of the polypeptide.

Macrophage: As used herein, the term “macrophage” refers to the major class of white blood cells in peripheral blood. Macrophages have an important role in engulfing and killing extracellular pathogens and removal of cellular debris.

scFv: As used herein, the term “scFv” refers to a fusion protein of the variable regions of the heavy (VH) and light chains (VL) of immunoglobulins, connected with a short linker peptide of ten to about 25 amino acids.

sdAb: As used herein, the term “sdAb” refers to a protein comprising an antibody fragment consisting of a single monomeric variable antibody domain, specifically the variable heavy (VH) domain. In some embodiments, a sdAb does not comprise an Fc region. In some embodiments, a sdAb is fused to an Fc region.

Fab: As used herein, the term “Fab” refers to an antibody fragment comprising a portion of an intact antibody, comprising the antigen-binding or variable region thereof.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Dysfunction: As used herein, the term “dysfunction” refers to an abnormal function. A dysfunction of a molecule (e.g, a protein) can be caused by an increase or decrease in activity associated with such molecule. A dysfunction of a molecule can be caused by a defect associated with the molecule itself, or other molecules that interact directly or indirectly with or regulate the molecule.

Derivatives: As used herein, the term “derivatives” when used in connection with antibody, or C5aR1 antibodies, refer to a portion having some of the sequence of an original molecule that retains at least some of the functions and/or properties of the original molecule.

Identity: As used herein, the term “identity” refers a relationship between the sequences of two or more polypeptide molecules or two or more nucleic acid molecules as known in the art, comparing the sequences of these molecules. The relationship determined by doing. In the art, “identity” also means the degree of sequence relatedness between nucleic acid molecules or polypeptides, and in some cases more than one nucleotide sequence or more than one. It may be determined by a match between amino acid sequence strings. “Identity” means between a gap alignment (if any) addressed by a particular mathematical model or computer program (i.e., an “algorithm”) and a smaller sequence of two or more sequences. Measure the percent identity match.

Similarity or Similar: As used herein, the term “similarity” is used in the art with respect to related concepts, but in contrast to “identity,” “similarity”, refers to both identity and conservative substitution matches. If two polypeptide sequences have, for example, 10 identical amino acids out of 20 amino acids and the rest are all non-conservative substitutions, the percent identity and percent similarity are both 50%. In the same example, if there are 5 more conservative substitutions, the percent identity remains 50%, but the percent similarity is 75%. Thus, if there are conservative substitutions, the percent similarity between the two polypeptides is higher than the percent identity between these two polypeptides.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Vector: The term “vector” refers to a polynucleotide (usually DNA) used to artificially carry foreign genetic material to another cell where it can be replicated or expressed. Non-limiting exemplary vectors include plasmids, viral vectors, cosmids, and artificial chromosomes. Such vectors may be derived from a variety of sources, including bacterial and viral sources. A non-limiting exemplary viral source for a plasmid is adeno-associated virus.

Various aspects of the disclosure are described in detail in the following sections. The use of sections is not meant to limit the disclosure. Each section can apply to any aspect of the disclosure. In this application, the use of “or” means “and/or” unless stated otherwise. As used herein, the singular forms “a”, “an”, and “the” include both singular and plural referents unless the context clearly dictates otherwise.

### DETAILED DESCRIPTION

The present invention provides, among other things, anti-CSF1R sdAbs that are characterized with high binding affinity to CSF1R (e.g., K_{D} less than 10 nM) and/or high inhibition activity of both CSF1 and IL-34 (e.g., IC₅₀ less than 15 nM). In some embodiments, the sdAb is fused to an Fc region. The anti-CSF1R sdAbs or sdAb-Fc fusions of the present invention can effectively inhibit binding of both CSF1R and IL-34 to CSFR1 or obstruct dimerization of CSF1R and thereby prevent CSF1R signaling.

### Function and Structure of CSF1R

Colony stimulating factor 1 receptor (CSF1R) is a class III receptor tyrosine kinase that is expressed on the cell surface of microglia and macrophages. CSF1R is directly involved in controlling development, survival and maintenance of microglia and these effects are associated with neuroinflammation.

CSF1R is activated by two distinct cytokines, colony-stimulating factor 1 (CSF-1) and interleukin-34 (IL-34) which, despite their disparate sequences, adopt similar four-helix bundle folds. While CSF-1 and IL-34 share similar biological activities they differ in their signaling patterns through CSF1R and in terms of spatiotemporal expression levels. The spatiotemporal expression of IL-34 differs from that of CSF-1, suggesting that they have distinct biologic functions. CSF-1 and the binding sites on the receptor have evolved rapidly across species. By contrast, IL-34 is much more conserved across species, and computational modeling suggests that it binds to different parts of the receptor. In keeping with this view, Chihara et al identified mAbs binding to CSF1R that can block CSF-1, but not IL-34 binding. The two proteins induced equivalent expression of chemokine genes when added to human whole blood, but Chihara et al found subtle difference in signal intensity between the two ligands on mouse cells. The very high level of conservation of IL-34 across species is atypical of immune-associated genes and could reflect an essential function.

CSF1R consists of an extracellular domain, a transmembrane helix, and an intracellular cytoplasmic domain. The extracellular part includes five immunoglobulin-like domains (D1-D5), which contribute to ligand binding and receptor stabilization. The three N-terminal Ig domains (D1-D3) are primarily responsible for ligand recognition, allowing CSF1R to bind its ligands, such as CSF-1 and IL-34. The subsequent two Ig domains (D4-D5) play a role in stabilizing the ligand-receptor complex, ensuring effective signal transduction. This structural arrangement facilitates receptor dimerization and autophosphorylation, which activate downstream signaling pathways that regulate myeloid cell functions.

Anti-CSF1R Single-Domain Antibodies (sdAbs)

A single domain antibody (sdAb), VHH, or a nanobody is a fragment represents the smallest antigen binding domains of antibodies. Despite having a molecular weight typically less than its parent antibody of 12-15 KDa, this type of antibody retains its binding specificity and affinity. This type of single-domain antibody is characterized by its high physical and thermal stability and production process.

In some embodiments, a first sdAb fused to a second sdAb. In some embodiments, the first sdAb comprises the same amino acid sequence as the second sdAb. In some embodiments, the first sdAb comprises a different amino acid sequence from the second sdAb.

Sequences of Exemplary Anti-CSF1R sdAbs

TABLE A
Exemplary Llama anti-CSF1R VHH Sequences

| Antibody | | SEQ |
|---|---|---|
| (Ab) | Sequence | ID NO |
| Ab1 | EVQLQESGGGLVQAGGSLRLSCAASGGTFSYYNMGWFRQAPGKEREFVAVISQYGSNTYYA | 1 |
| | DSVKGRFTITRDNAKNAAYLQLNSLEPEDTAVYYCATLRSGSDWRTIRDYDNWGQGTQVTVSS | |
| Ab2 | EVQLQESGGGLVQPGGSLRLSCAASGDIFSFNVMGWFRQAPGKERELVATIAPGGNTNYFDS | 2 |
| | ARGRFTISRDSAKNTVYLQMNSLKPEDTAVYYCNRWPFSRDSWGQGTQVTVSS | |
| Ab3 | EVQLQESGGGLVQAGGSLRLSCVASGMAFGSSPMNWVRQAPGKEPEWVSGVTSAGYAYYA | 3 |
| | DWVKGRFTASRDNAKNTLFLQMNSLKPEDTAVYYCARGPFHSTAYSTRGQGTQVTVSS | |
| Ab4 | EVQLQESGGGLVQPGDSLRLSCVGSGLTFGSSLMNWVRQAPGKGPEWVGGVTSGGYSYYA | 4 |
| | ESVKGRFTISRDNAKNTLFLQMNSLKPEDTAVYYCARGPFHSIAYATRGQGTQVTVSS | |
| Ab5 | EVQLQESGGGLVQPGDSLRLSCVGSGFTFGSTLMNWVRQAPGKGPEWVGGVTSGGYSYYA | 5 |
| | DSVKGRFTISRDNAKNTLFLQMNSLKPEDTAVYYCARGPFHSIAYATRGQGTQVTVSS | |
| Ab6 | EVQLQESGGGLVQAGGSLRLSCAASGLTFSYYNMGWFRQAPGKEREFVAVISQYGSNTYYAD | 6 |
| | SVKGRFTITRDNAKNAAYLQLNSLKPEDTAVYYCATLRSGSDWRTMRDYDNWGQGTQVTVSS | |
| Ab7 | EVQLQESGGGLVQPGGSLRLSCAASGNIFSFNVMGWFRQAPGKERELVATIAPGGKTNYADP | 7 |
| | AKGRFTISRDSAKNTVYLQMNSLKPEDTAVYYCNRWPFSRDSWGQGTQVTVSS | |
| Ab8 | EVQLQESGGGLVQAGGSLRLSCAASGMAFGSSPMNWVRQAPGKEPEWVSGVTSAGYAYYA | 8 |
| | DWVKGRFTASRDNAKNTLFLQMNSLKPEDTAVYYCARGPFHSTAYSTRGQGTQVTVSS | |
| Ab9 | EVQLQESGGGLVQPGGSLRLSCASSGNIFSFNVMGWFRQAPGKERELVATIAPGGKTNYADP | 9 |
| | AKGRFTISRDSAKNTVYLQMNSLKPEDTAVYYCNRWPFSRDSWGQGTQVTVSS | |
| Ab10 | EVQLQESGGGLVQPGGSLRLSCTASGNIFNINVMGWYRQAPGKERELLAHITSGGRVTYFDSA | 10 |
| | KGRFTITRDNAKNTMYLQMNSLKPEDTAVYYCNTLVHRRDSWGQGTQVTVSS | |
| Ab11 | EVQLQESGGGLMQAGGSLRLSCAASGDIFSFNVMGWFRQAPGKERELVATIAPGGNTNYFDS | 11 |
| | ARGRFTISRDSAKNTVYLQMNSLKPEDTAVYYCNRWPFSRDSWGQGTQVTVSS | |
| Ab12 | EVQLQESGGGLVQPGGSLRLSCAASKSTFSGNVIAWYRQAPGKQRELIAYITSGGKTLYADSA | 12 |
| | KGRFTISRDNAKNTVSLQMNSLEPEDTAVYYCNTLPFKDWGQGTQVTVSS | |
| Ab13 | EVQLQESGGGLVQPGGSLRLSCAASGDIFSFNVMGWFRQAPGKERELVATIAPGGKTNYADP | 13 |
| | AKGRFTISRDSAKNTVYLQMNSLKPEDTAVYYCNRWPFSRDSWGQGTQVTVSS | |
| Ab14 | EVQLQESGGGLVQPGGSLRLSCAASENIFRSTVMGWYRQAPGKERELLAHITSGGRAQYFDS | 14 |
| | AKGRFTITRDNAKNTVFLQMNSLKPEDTAVYYCNSLVHRRDSWGQGTQVTVSS | |
| Ab15 | EVQLQESGGGLVQPGGSLRLSCAASGFTFGSTTMNWVRQAPGKGPEWVGGVTSSGYSYYA | 15 |
| | DSVKGRFTISRDNAKNTLFLQMNSLKFEDTAVYYCARGPFHSTAYATRGQGTQVTVSS | |
| Ab16 | EVQLQESGGGLVQAGGSLRLSCAASGMSFGPNVIGWFRQAPGKQRELVAHITSGGNTLYADS | 16 |
| | AKGRFTISRDNAANTVYLQMNSLKSEDTAVYYCNTLPWTRGSWGQGTQVTVSS | |
| Ab17 | EVQLQESGGGLVQAGGSLRLSCAASGNTGRYNVFGWFRQAPGKQRELVAHITTGGRTFYSD | 17 |
| | AGKGRFTITRNNAENTVYLQMNSLKPEDTAVYYCNSLPFEAHSWGQGTQVTVSS | |
| Ab18 | EVQLQESGGGLVQPGGSLRLSCAASGNIFRSNVMGWYRQAPGKERELLAHITSGGKTQYFDS | 18 |
| | AKGRFTITRDNAKNTVYLQMNSLKPEDTAVYYCNSLVHRRDSWGQGTQVTVSS | |
| Ab19 | EVQLQESGGGLVQLGGSLRLSCAASGDIFSFNVMGWFRQAPGKERELVATIAPGGNTNYFDS | 19 |
| | ARGRFTISRDSAKNTVYLQMNSLKPEDTAVYYCNRWPFSRDSWGQGTQVTVSS | |
| Ab20 | EVQLQESGGGLVQPGGSLRLSCTASRDIFSFNIMAWYRQAPGKERELVARITTGGITWYADPG | 20 |
| | QGRFTISRDNVENTVYLQMNSLKPEDTAVYYCNTLPFRPTYWGQGTQVTVSS | |
| Ab21 | EVQLQESGGGLVQPGGSLRLSCVASGNIFRSNVMGWYRQAPGKERELLAHITSDGRTQYFDS | 21 |
| | AKGRFTITRDNAKNTVYLQMNSLKPEDTAVYYCNSLVHRRDSWGQGTQVTVSS | |
| Ab22 | EVQLQESGGGLVQPGGSLRLSCVASGNIFRSNVMGWYRQAPGKERELLAHITSGGSTQYFDS | 22 |
| | AKGRFTITRDNAKNTVYLQMNSLKPEDTAIYYCNSLVHRRDSWGQGTQVTVSS | |
| Ab23 | EVQLQESGGGLVQPGGSLRLSCAAPRSIFVFNIMGWYRQAPGKERELVAHISSGGNTFYSSSV | 23 |
| | EGRFTISRDTAKNTVYLQMNSLKPEDTAVYYCNTLPFRPASWGQGTQVTVSS | |
| Ab24 | EVQLQESGGGLVQPGGSLRLSCAASGMAFGSSPMNWVRQAPGKGPEWVSGTTSAGYTYYA | 24 |
| | DSVKGRFTASRDNAKNTLFLQMNSLEPEDTAVYYCARGPFHSTAYSTRGQGTQVTVSS | |
| Ab25 | EVQLQESGGGLVQPGGSLRLSCAASGSIFSGNVMGWFRQAPGKEREAVAWLTPDGSRTYYA | 25 |
| | DSAKGRFTISRDNTKNTVYLQMNSLKPEDTAVYYCNKLPFSSWGQGTQVTVSS | |

In some embodiments, an anti-CSF1R sdAb comprises an amino acid sequence that is at least 80%, 82%, 85%, 88%, 90%, 91%, 92%, 93%, 94%, 95%, 96%, 97%, 98%, or 99% identical to any one of SEQ ID NOs: 1-25 as shown in Table A. In some embodiments, an anti-CSF1R sdAb comprises an amino acid sequence of any one of SEQ ID NOs: 1-25

In some embodiments, an anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 1. In some embodiments, the anti-CSF1R sdAb comprises an amino acid sequence that is at least 80% identical to SEQ ID NO: 1. In some embodiments, the anti-CSF1R sdAb comprises an amino acid sequence that is at least 82% identical to SEQ ID NO: 1. In some embodiments, the anti-CSF1R sdAb comprises an amino acid sequence that is at least 85% identical to SEQ ID NO: 1. In some embodiments, the anti-CSF1R sdAb comprises an amino acid sequence that is at least 88% identical to SEQ ID NO: 1. In some embodiments, the anti-CSF1R sdAb comprises an amino acid sequence that is at least 90% identical to SEQ ID NO: 1. In some embodiments, the anti-CSF1R sdAb comprises an amino acid sequence that is at least 91% identical to SEQ ID NO: 1. In some embodiments, the anti-CSF1R sdAb comprises an amino acid sequence that is at least 92% identical to SEQ ID NO: 1. In some embodiments, the anti-CSF1R sdAb comprises an amino acid sequence that is at least 93% identical to SEQ ID NO: 1. In some embodiments, the anti-CSF1R sdAb comprises an amino acid sequence that is at least 94% identical to SEQ ID NO: 1. In some embodiments, the anti-CSF1R sdAb comprises an amino acid sequence that is at least 95% identical to SEQ ID NO: 1. In some embodiments, the anti-CSF1R sdAb comprises an amino acid sequence that is at least 96% identical to SEQ ID NO: 1. In some embodiments, the anti-CSF1R sdAb comprises an amino acid sequence that is at least 97% identical to SEQ ID NO: 1. In some embodiments, the anti-CSF1R sdAb comprises an amino acid sequence that is at least 98% identical to SEQ ID NO: 1. In some embodiments, the anti-CSF1R sdAb comprises an amino acid sequence that is at least 99% identical to SEQ ID NO: 1. In some embodiments, the anti-CSF1R sdAb comprises an amino acid sequence of SEQ ID NO: 1.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 2. In some embodiments, the anti-CSF1R sdAb comprises an amino acid sequence that is at least 80% identical to SEQ ID NO: 2. In some embodiments, the anti-CSF1R sdAb comprises an amino acid sequence that is at least 82% identical to SEQ ID NO: 2. In some embodiments, the anti-CSF1R sdAb comprises an amino acid sequence that is at least 85% identical to SEQ ID NO: 2. In some embodiments, the anti-CSF1R sdAb comprises an amino acid sequence that is at least 88% identical to SEQ ID NO: 2. In some embodiments, the anti-CSF1R sdAb comprises an amino acid sequence that is at least 90% identical to SEQ ID NO: 2. In some embodiments, the anti-CSF1R sdAb comprises an amino acid sequence that is at least 91% identical to SEQ ID NO: 2. In some embodiments, the anti-CSF1R sdAb comprises an amino acid sequence that is at least 92% identical to SEQ ID NO: 2. In some embodiments, the anti-CSF1R sdAb comprises an amino acid sequence that is at least 93% identical to SEQ ID NO: 2. In some embodiments, the anti-CSF1R sdAb comprises an amino acid sequence that is at least 94% identical to SEQ ID NO: 2. In some embodiments, the anti-CSF1R sdAb comprises an amino acid sequence that is at least 95% identical to SEQ ID NO: 2. In some embodiments, the anti-CSF1R sdAb comprises an amino acid sequence that is at least 96% identical to SEQ ID NO: 2. In some embodiments, the anti-CSF1R sdAb comprises an amino acid sequence that is at least 97% identical to SEQ ID NO: 2. In some embodiments, the anti-CSF1R sdAb comprises an amino acid sequence that is at least 98% identical to SEQ ID NO: 2. In some embodiments, the anti-CSF1R sdAb comprises an amino acid sequence that is at least 99% identical to SEQ ID NO: 2. In some embodiments, the anti-CSF1R sdAb comprises an amino acid sequence of SEQ ID NO: 2.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 3. In some embodiments, the anti-CSF1R sdAb comprises an amino acid sequence that is at least 80% identical to SEQ ID NO: 3. In some embodiments, the anti-CSF1R sdAb comprises an amino acid sequence that is at least 82% identical to SEQ ID NO: 3. In some embodiments, the anti-CSF1R sdAb comprises an amino acid sequence that is at least 85% identical to SEQ ID NO: 3. In some embodiments, the anti-CSF1R sdAb comprises an amino acid sequence that is at least 88% identical to SEQ ID NO: 3. In some embodiments, the anti-CSF1R sdAb comprises an amino acid sequence that is at least 90% identical to SEQ ID NO: 3. In some embodiments, the anti-CSF1R sdAb comprises an amino acid sequence that is at least 91% identical to SEQ ID NO: 3. In some embodiments, the anti-CSF1R sdAb comprises an amino acid sequence that is at least 92% identical to SEQ ID NO: 3. In some embodiments, the anti-CSF1R sdAb comprises an amino acid sequence that is at least 93% identical to SEQ ID NO: 3. In some embodiments, the anti-CSF1R sdAb comprises an amino acid sequence that is at least 94% identical to SEQ ID NO: 3. In some embodiments, the anti-CSF1R sdAb comprises an amino acid sequence that is at least 95% identical to SEQ ID NO: 3. In some embodiments, the anti-CSF1R sdAb comprises an amino acid sequence that is at least 96% identical to SEQ ID NO: 3. In some embodiments, the anti-CSF1R sdAb comprises an amino acid sequence that is at least 97% identical to SEQ ID NO: 3. In some embodiments, the anti-CSF1R sdAb comprises an amino acid sequence that is at least 98% identical to SEQ ID NO: 3. In some embodiments, the anti-CSF1R sdAb comprises an amino acid sequence that is at least 99% identical to SEQ ID NO: 3. In some embodiments, the anti-CSF1R sdAb comprises an amino acid sequence of SEQ ID NO: 3.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 4. In some embodiments, the anti-CSF1R sdAb comprises an amino acid sequence that is at least 80% identical to SEQ ID NO: 4. In some embodiments, the anti-CSF1R sdAb comprises an amino acid sequence that is at least 82% identical to SEQ ID NO: 4. In some embodiments, the anti-CSF1R sdAb comprises an amino acid sequence that is at least 85% identical to SEQ ID NO: 4. In some embodiments, the anti-CSF1R sdAb comprises an amino acid sequence that is at least 88% identical to SEQ ID NO: 4. In some embodiments, the anti-CSF1R sdAb comprises an amino acid sequence that is at least 90% identical to SEQ ID NO: 4. In some embodiments, the anti-CSF1R sdAb comprises an amino acid sequence that is at least 91% identical to SEQ ID NO: 4. In some embodiments, the anti-CSF1R sdAb comprises an amino acid sequence that is at least 92% identical to SEQ ID NO: 4. In some embodiments, the anti-CSF1R sdAb comprises an amino acid sequence that is at least 93% identical to SEQ ID NO: 4. In some embodiments, the anti-CSF1R sdAb comprises an amino acid sequence that is at least 94% identical to SEQ ID NO: 4. In some embodiments, the anti-CSF1R sdAb comprises an amino acid sequence that is at least 95% identical to SEQ ID NO: 4. In some embodiments, the anti-CSF1R sdAb comprises an amino acid sequence that is at least 96% identical to SEQ ID NO: 4. In some embodiments, the anti-CSF1R sdAb comprises an amino acid sequence that is at least 97% identical to SEQ ID NO: 4. In some embodiments, the anti-CSF1R sdAb comprises an amino acid sequence that is at least 98% identical to SEQ ID NO: 4. In some embodiments, the anti-CSF1R sdAb comprises an amino acid sequence that is at least 99% identical to SEQ ID NO: 4. In some embodiments, the anti-CSF1R sdAb comprises an amino acid sequence of SEQ ID NO: 4.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 5. In some embodiments, the anti-CSF1R sdAb comprises an amino acid sequence that is at least 80% identical to SEQ ID NO: 5. In some embodiments, the anti-CSF1R sdAb comprises an amino acid sequence that is at least 82% identical to SEQ ID NO: 5. In some embodiments, the anti-CSF1R sdAb comprises an amino acid sequence that is at least 85% identical to SEQ ID NO: 5. In some embodiments, the anti-CSF1R sdAb comprises an amino acid sequence that is at least 88% identical to SEQ ID NO: 5. In some embodiments, the anti-CSF1R sdAb comprises an amino acid sequence that is at least 90% identical to SEQ ID NO: 5. In some embodiments, the anti-CSF1R sdAb comprises an amino acid sequence that is at least 91% identical to SEQ ID NO: 5. In some embodiments, the anti-CSF1R sdAb comprises an amino acid sequence that is at least 92% identical to SEQ ID NO: 5. In some embodiments, the anti-CSF1R sdAb comprises an amino acid sequence that is at least 93% identical to SEQ ID NO: 5. In some embodiments, the anti-CSF1R sdAb comprises an amino acid sequence that is at least 94% identical to SEQ ID NO: 5. In some embodiments, the anti-CSF1R sdAb comprises an amino acid sequence that is at least 95% identical to SEQ ID NO: 5. In some embodiments, the anti-CSF1R sdAb comprises an amino acid sequence that is at least 96% identical to SEQ ID NO: 5. In some embodiments, the anti-CSF1R sdAb comprises an amino acid sequence that is at least 97% identical to SEQ ID NO: 5. In some embodiments, the anti-CSF1R sdAb comprises an amino acid sequence that is at least 98% identical to SEQ ID NO: 5. In some embodiments, the anti-CSF1R sdAb comprises an amino acid sequence that is at least 99% identical to SEQ ID NO: 5. In some embodiments, the anti-CSF1R sdAb comprises an amino acid sequence of SEQ ID NO: 5.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 6. In some embodiments, the anti-CSF1R sdAb comprises an amino acid sequence that is at least 80% identical to SEQ ID NO: 6. In some embodiments, the anti-CSF1R sdAb comprises an amino acid sequence that is at least 82% identical to SEQ ID NO: 6. In some embodiments, the anti-CSF1R sdAb comprises an amino acid sequence that is at least 85% identical to SEQ ID NO: 6. In some embodiments, the anti-CSF1R sdAb comprises an amino acid sequence that is at least 88% identical to SEQ ID NO: 6. In some embodiments, the anti-CSF1R sdAb comprises an amino acid sequence that is at least 90% identical to SEQ ID NO: 6. In some embodiments, the anti-CSF1R sdAb comprises an amino acid sequence that is at least 91% identical to SEQ ID NO: 6. In some embodiments, the anti-CSF1R sdAb comprises an amino acid sequence that is at least 92% identical to SEQ ID NO: 6. In some embodiments, the anti-CSF1R sdAb comprises an amino acid sequence that is at least 93% identical to SEQ ID NO: 6. In some embodiments, the anti-CSF1R sdAb comprises an amino acid sequence that is at least 94% identical to SEQ ID NO: 6. In some embodiments, the anti-CSF1R sdAb comprises an amino acid sequence that is at least 95% identical to SEQ ID NO: 6. In some embodiments, the anti-CSF1R sdAb comprises an amino acid sequence that is at least 96% identical to SEQ ID NO: 6. In some embodiments, the anti-CSF1R sdAb comprises an amino acid sequence that is at least 97% identical to SEQ ID NO: 6. In some embodiments, the anti-CSF1R sdAb comprises an amino acid sequence that is at least 98% identical to SEQ ID NO: 6. In some embodiments, the anti-CSF1R sdAb comprises an amino acid sequence that is at least 99% identical to SEQ ID NO: 6. In some embodiments, the anti-CSF1R sdAb comprises an amino acid sequence of SEQ ID NO: 6.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 7. In some embodiments, the anti-CSF1R sdAb comprises an amino acid sequence that is at least 80% identical to SEQ ID NO: 7. In some embodiments, the anti-CSF1R sdAb comprises an amino acid sequence that is at least 82% identical to SEQ ID NO: 7. In some embodiments, the anti-CSF1R sdAb comprises an amino acid sequence that is at least 85% identical to SEQ ID NO: 7. In some embodiments, the anti-CSF1R sdAb comprises an amino acid sequence that is at least 88% identical to SEQ ID NO: 7. In some embodiments, the anti-CSF1R sdAb comprises an amino acid sequence that is at least 90% identical to SEQ ID NO: 7. In some embodiments, the anti-CSF1R sdAb comprises an amino acid sequence that is at least 91% identical to SEQ ID NO: 7. In some embodiments, the anti-CSF1R sdAb comprises an amino acid sequence that is at least 92% identical to SEQ ID NO: 7. In some embodiments, the anti-CSF1R sdAb comprises an amino acid sequence that is at least 93% identical to SEQ ID NO: 7. In some embodiments, the anti-CSF1R sdAb comprises an amino acid sequence that is at least 94% identical to SEQ ID NO: 7. In some embodiments, the anti-CSF1R sdAb comprises an amino acid sequence that is at least 95% identical to SEQ ID NO: 7. In some embodiments, the anti-CSF1R sdAb comprises an amino acid sequence that is at least 96% identical to SEQ ID NO: 7. In some embodiments, the anti-CSF1R sdAb comprises an amino acid sequence that is at least 97% identical to SEQ ID NO: 7. In some embodiments, the anti-CSF1R sdAb comprises an amino acid sequence that is at least 98% identical to SEQ ID NO: 7. In some embodiments, the anti-CSF1R sdAb comprises an amino acid sequence that is at least 99% identical to SEQ ID NO: 7. In some embodiments, the anti-CSF1R sdAb comprises an amino acid sequence of SEQ ID NO: 7.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 8. In some embodiments, the anti-CSF1R sdAb comprises an amino acid sequence that is at least 80% identical to SEQ ID NO: 8. In some embodiments, the anti-CSF1R sdAb comprises an amino acid sequence that is at least 82% identical to SEQ ID NO: 8. In some embodiments, the anti-CSF1R sdAb comprises an amino acid sequence that is at least 85% identical to SEQ ID NO: 8. In some embodiments, the anti-CSF1R sdAb comprises an amino acid sequence that is at least 88% identical to SEQ ID NO: 8. In some embodiments, the anti-CSF1R sdAb comprises an amino acid sequence that is at least 90% identical to SEQ ID NO: 8. In some embodiments, the anti-CSF1R sdAb comprises an amino acid sequence that is at least 91% identical to SEQ ID NO: 8. In some embodiments, the anti-CSF1R sdAb comprises an amino acid sequence that is at least 92% identical to SEQ ID NO: 8. In some embodiments, the anti-CSF1R sdAb comprises an amino acid sequence that is at least 93% identical to SEQ ID NO: 8. In some embodiments, the anti-CSF1R sdAb comprises an amino acid sequence that is at least 94% identical to SEQ ID NO: 8. In some embodiments, the anti-CSF1R sdAb comprises an amino acid sequence that is at least 95% identical to SEQ ID NO: 8. In some embodiments, the anti-CSF1R sdAb comprises an amino acid sequence that is at least 96% identical to SEQ ID NO: 8. In some embodiments, the anti-CSF1R sdAb comprises an amino acid sequence that is at least 97% identical to SEQ ID NO: 8. In some embodiments, the anti-CSF1R sdAb comprises an amino acid sequence that is at least 98% identical to SEQ ID NO: 8. In some embodiments, the anti-CSF1R sdAb comprises an amino acid sequence that is at least 99% identical to SEQ ID NO: 8. In some embodiments, the anti-CSF1R sdAb comprises an amino acid sequence of SEQ ID NO: 8.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 9. In some embodiments, the anti-CSF1R sdAb comprises an amino acid sequence that is at least 80% identical to SEQ ID NO: 9. In some embodiments, the anti-CSF1R sdAb comprises an amino acid sequence that is at least 82% identical to SEQ ID NO: 9. In some embodiments, the anti-CSF1R sdAb comprises an amino acid sequence that is at least 85% identical to SEQ ID NO: 9. In some embodiments, the anti-CSF1R sdAb comprises an amino acid sequence that is at least 88% identical to SEQ ID NO: 9. In some embodiments, the anti-CSF1R sdAb comprises an amino acid sequence that is at least 90% identical to SEQ ID NO: 9. In some embodiments, the anti-CSF1R sdAb comprises an amino acid sequence that is at least 91% identical to SEQ ID NO: 9. In some embodiments, the anti-CSF1R sdAb comprises an amino acid sequence that is at least 92% identical to SEQ ID NO: 9. In some embodiments, the anti-CSF1R sdAb comprises an amino acid sequence that is at least 93% identical to SEQ ID NO: 9. In some embodiments, the anti-CSF1R sdAb comprises an amino acid sequence that is at least 94% identical to SEQ ID NO: 9. In some embodiments, the anti-CSF1R sdAb comprises an amino acid sequence that is at least 95% identical to SEQ ID NO: 9. In some embodiments, the anti-CSF1R sdAb comprises an amino acid sequence that is at least 96% identical to SEQ ID NO: 9. In some embodiments, the anti-CSF1R sdAb comprises an amino acid sequence that is at least 97% identical to SEQ ID NO: 9. In some embodiments, the anti-CSF1R sdAb comprises an amino acid sequence that is at least 98% identical to SEQ ID NO: 9. In some embodiments, the anti-CSF1R sdAb comprises an amino acid sequence that is at least 99% identical to SEQ ID NO: 9. In some embodiments, the anti-CSF1R sdAb comprises an amino acid sequence of SEQ ID NO: 9.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 10. In some embodiments, the anti-CSF1R sdAb comprises an amino acid sequence that is at least 80% identical to SEQ ID NO: 10. In some embodiments, the anti-CSF1R sdAb comprises an amino acid sequence that is at least 82% identical to SEQ ID NO: 10. In some embodiments, the anti-CSF1R sdAb comprises an amino acid sequence that is at least 85% identical to SEQ ID NO: 10. In some embodiments, the anti-CSF1R sdAb comprises an amino acid sequence that is at least 88% identical to SEQ ID NO: 10. In some embodiments, the anti-CSF1R sdAb comprises an amino acid sequence that is at least 90% identical to SEQ ID NO: 10. In some embodiments, the anti-CSF1R sdAb comprises an amino acid sequence that is at least 91% identical to SEQ ID NO: 10. In some embodiments, the anti-CSF1R sdAb comprises an amino acid sequence that is at least 92% identical to SEQ ID NO: 10. In some embodiments, the anti-CSF1R sdAb comprises an amino acid sequence that is at least 93% identical to SEQ ID NO: 10. In some embodiments, the anti-CSF1R sdAb comprises an amino acid sequence that is at least 94% identical to SEQ ID NO: 10. In some embodiments, the anti-CSF1R sdAb comprises an amino acid sequence that is at least 95% identical to SEQ ID NO: 10. In some embodiments, the anti-CSF1R sdAb comprises an amino acid sequence that is at least 96% identical to SEQ ID NO: 10. In some embodiments, the anti-CSF1R sdAb comprises an amino acid sequence that is at least 97% identical to SEQ ID NO: 10. In some embodiments, the anti-CSF1R sdAb comprises an amino acid sequence that is at least 98% identical to SEQ ID NO: 10. In some embodiments, the anti-CSF1R sdAb comprises an amino acid sequence that is at least 99% identical to SEQ ID NO: 10. In some embodiments, the anti-CSF1R sdAb comprises an amino acid sequence of SEQ ID NO: 10.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 11. In some embodiments, the anti-CSF1R sdAb comprises an amino acid sequence that is at least 80% identical to SEQ ID NO: 11. In some embodiments, the anti-CSF1R sdAb comprises an amino acid sequence that is at least 82% identical to SEQ ID NO: 11. In some embodiments, the anti-CSF1R sdAb comprises an amino acid sequence that is at least 85% identical to SEQ ID NO: 11. In some embodiments, the anti-CSF1R sdAb comprises an amino acid sequence that is at least 88% identical to SEQ ID NO: 11. In some embodiments, the anti-CSF1R sdAb comprises an amino acid sequence that is at least 90% identical to SEQ ID NO: 11. In some embodiments, the anti-CSF1R sdAb comprises an amino acid sequence that is at least 91% identical to SEQ ID NO: 11. In some embodiments, the anti-CSF1R sdAb comprises an amino acid sequence that is at least 92% identical to SEQ ID NO: 11. In some embodiments, the anti-CSF1R sdAb comprises an amino acid sequence that is at least 93% identical to SEQ ID NO: 11. In some embodiments, the anti-CSF1R sdAb comprises an amino acid sequence that is at least 94% identical to SEQ ID NO: 11. In some embodiments, the anti-CSF1R sdAb comprises an amino acid sequence that is at least 95% identical to SEQ ID NO: 11. In some embodiments, the anti-CSF1R sdAb comprises an amino acid sequence that is at least 96% identical to SEQ ID NO: 11. In some embodiments, the anti-CSF1R sdAb comprises an amino acid sequence that is at least 97% identical to SEQ ID NO: 11. In some embodiments, the anti-CSF1R sdAb comprises an amino acid sequence that is at least 98% identical to SEQ ID NO: 11. In some embodiments, the anti-CSF1R sdAb comprises an amino acid sequence that is at least 99% identical to SEQ ID NO: 11. In some embodiments, the anti-CSF1R sdAb comprises an amino acid sequence of SEQ ID NO: 11.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 12. In some embodiments, the anti-CSF1R sdAb comprises an amino acid sequence that is at least 80% identical to SEQ ID NO: 12. In some embodiments, the anti-CSF1R sdAb comprises an amino acid sequence that is at least 82% identical to SEQ ID NO: 12. In some embodiments, the anti-CSF1R sdAb comprises an amino acid sequence that is at least 85% identical to SEQ ID NO: 12. In some embodiments, the anti-CSF1R sdAb comprises an amino acid sequence that is at least 88% identical to SEQ ID NO: 12. In some embodiments, the anti-CSF1R sdAb comprises an amino acid sequence that is at least 90% identical to SEQ ID NO: 12. In some embodiments, the anti-CSF1R sdAb comprises an amino acid sequence that is at least 91% identical to SEQ ID NO: 12. In some embodiments, the anti-CSF1R sdAb comprises an amino acid sequence that is at least 92% identical to SEQ ID NO: 12. In some embodiments, the anti-CSF1R sdAb comprises an amino acid sequence that is at least 93% identical to SEQ ID NO: 12. In some embodiments, the anti-CSF1R sdAb comprises an amino acid sequence that is at least 94% identical to SEQ ID NO: 12. In some embodiments, the anti-CSF1R sdAb comprises an amino acid sequence that is at least 95% identical to SEQ ID NO: 12. In some embodiments, the anti-CSF1R sdAb comprises an amino acid sequence that is at least 96% identical to SEQ ID NO: 12. In some embodiments, the anti-CSF1R sdAb comprises an amino acid sequence that is at least 97% identical to SEQ ID NO: 12. In some embodiments, the anti-CSF1R sdAb comprises an amino acid sequence that is at least 98% identical to SEQ ID NO: 12. In some embodiments, the anti-CSF1R sdAb comprises an amino acid sequence that is at least 99% identical to SEQ ID NO: 12. In some embodiments, the anti-CSF1R sdAb comprises an amino acid sequence of SEQ ID NO: 12.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 13. In some embodiments, the anti-CSF1R sdAb comprises an amino acid sequence that is at least 80% identical to SEQ ID NO: 13. In some embodiments, the anti-CSF1R sdAb comprises an amino acid sequence that is at least 82% identical to SEQ ID NO: 13. In some embodiments, the anti-CSF1R sdAb comprises an amino acid sequence that is at least 85% identical to SEQ ID NO: 13. In some embodiments, the anti-CSF1R sdAb comprises an amino acid sequence that is at least 88% identical to SEQ ID NO: 13. In some embodiments, the anti-CSF1R sdAb comprises an amino acid sequence that is at least 90% identical to SEQ ID NO: 13. In some embodiments, the anti-CSF1R sdAb comprises an amino acid sequence that is at least 91% identical to SEQ ID NO: 13. In some embodiments, the anti-CSF1R sdAb comprises an amino acid sequence that is at least 92% identical to SEQ ID NO: 13. In some embodiments, the anti-CSF1R sdAb comprises an amino acid sequence that is at least 93% identical to SEQ ID NO: 13. In some embodiments, the anti-CSF1R sdAb comprises an amino acid sequence that is at least 94% identical to SEQ ID NO: 13. In some embodiments, the anti-CSF1R sdAb comprises an amino acid sequence that is at least 95% identical to SEQ ID NO: 13. In some embodiments, the anti-CSF1R sdAb comprises an amino acid sequence that is at least 96% identical to SEQ ID NO: 13. In some embodiments, the anti-CSF1R sdAb comprises an amino acid sequence that is at least 97% identical to SEQ ID NO: 13. In some embodiments, the anti-CSF1R sdAb comprises an amino acid sequence that is at least 98% identical to SEQ ID NO: 13. In some embodiments, the anti-CSF1R sdAb comprises an amino acid sequence that is at least 99% identical to SEQ ID NO: 13. In some embodiments, the anti-CSF1R sdAb comprises an amino acid sequence of SEQ ID NO: 13.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 14. In some embodiments, the anti-CSF1R sdAb comprises an amino acid sequence that is at least 80% identical to SEQ ID NO: 14. In some embodiments, the anti-CSF1R sdAb comprises an amino acid sequence that is at least 82% identical to SEQ ID NO: 14. In some embodiments, the anti-CSF1R sdAb comprises an amino acid sequence that is at least 85% identical to SEQ ID NO: 14. In some embodiments, the anti-CSF1R sdAb comprises an amino acid sequence that is at least 88% identical to SEQ ID NO: 14. In some embodiments, the anti-CSF1R sdAb comprises an amino acid sequence that is at least 90% identical to SEQ ID NO: 14. In some embodiments, the anti-CSF1R sdAb comprises an amino acid sequence that is at least 91% identical to SEQ ID NO: 14. In some embodiments, the anti-CSF1R sdAb comprises an amino acid sequence that is at least 92% identical to SEQ ID NO: 14. In some embodiments, the anti-CSF1R sdAb comprises an amino acid sequence that is at least 93% identical to SEQ ID NO: 14. In some embodiments, the anti-CSF1R sdAb comprises an amino acid sequence that is at least 94% identical to SEQ ID NO: 14. In some embodiments, the anti-CSF1R sdAb comprises an amino acid sequence that is at least 95% identical to SEQ ID NO: 14. In some embodiments, the anti-CSF1R sdAb comprises an amino acid sequence that is at least 96% identical to SEQ ID NO: 14. In some embodiments, the anti-CSF1R sdAb comprises an amino acid sequence that is at least 97% identical to SEQ ID NO: 14. In some embodiments, the anti-CSF1R sdAb comprises an amino acid sequence that is at least 98% identical to SEQ ID NO: 14. In some embodiments, the anti-CSF1R sdAb comprises an amino acid sequence that is at least 99% identical to SEQ ID NO: 14. In some embodiments, the anti-CSF1R sdAb comprises an amino acid sequence of SEQ ID NO: 14.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 15. In some embodiments, the anti-CSF1R sdAb comprises an amino acid sequence that is at least 80% identical to SEQ ID NO: 15. In some embodiments, the anti-CSF1R sdAb comprises an amino acid sequence that is at least 82% identical to SEQ ID NO: 15. In some embodiments, the anti-CSF1R sdAb comprises an amino acid sequence that is at least 85% identical to SEQ ID NO: 15. In some embodiments, the anti-CSF1R sdAb comprises an amino acid sequence that is at least 88% identical to SEQ ID NO: 15. In some embodiments, the anti-CSF1R sdAb comprises an amino acid sequence that is at least 90% identical to SEQ ID NO: 15. In some embodiments, the anti-CSF1R sdAb comprises an amino acid sequence that is at least 91% identical to SEQ ID NO: 15. In some embodiments, the anti-CSF1R sdAb comprises an amino acid sequence that is at least 92% identical to SEQ ID NO: 15. In some embodiments, the anti-CSF1R sdAb comprises an amino acid sequence that is at least 93% identical to SEQ ID NO: 15. In some embodiments, the anti-CSF1R sdAb comprises an amino acid sequence that is at least 94% identical to SEQ ID NO: 15. In some embodiments, the anti-CSF1R sdAb comprises an amino acid sequence that is at least 95% identical to SEQ ID NO: 15. In some embodiments, the anti-CSF1R sdAb comprises an amino acid sequence that is at least 96% identical to SEQ ID NO: 15. In some embodiments, the anti-CSF1R sdAb comprises an amino acid sequence that is at least 97% identical to SEQ ID NO: 15. In some embodiments, the anti-CSF1R sdAb comprises an amino acid sequence that is at least 98% identical to SEQ ID NO: 15. In some embodiments, the anti-CSF1R sdAb comprises an amino acid sequence that is at least 99% identical to SEQ ID NO: 15. In some embodiments, the anti-CSF1R sdAb comprises an amino acid sequence of SEQ ID NO: 15.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 16. In some embodiments, the anti-CSF1R sdAb comprises an amino acid sequence that is at least 80% identical to SEQ ID NO: 16. In some embodiments, the anti-CSF1R sdAb comprises an amino acid sequence that is at least 82% identical to SEQ ID NO: 16. In some embodiments, the anti-CSF1R sdAb comprises an amino acid sequence that is at least 85% identical to SEQ ID NO: 16. In some embodiments, the anti-CSF1R sdAb comprises an amino acid sequence that is at least 88% identical to SEQ ID NO: 16. In some embodiments, the anti-CSF1R sdAb comprises an amino acid sequence that is at least 90% identical to SEQ ID NO: 16. In some embodiments, the anti-CSF1R sdAb comprises an amino acid sequence that is at least 91% identical to SEQ ID NO: 16. In some embodiments, the anti-CSF1R sdAb comprises an amino acid sequence that is at least 92% identical to SEQ ID NO: 16. In some embodiments, the anti-CSF1R sdAb comprises an amino acid sequence that is at least 93% identical to SEQ ID NO: 16. In some embodiments, the anti-CSF1R sdAb comprises an amino acid sequence that is at least 94% identical to SEQ ID NO: 16. In some embodiments, the anti-CSF1R sdAb comprises an amino acid sequence that is at least 95% identical to SEQ ID NO: 16. In some embodiments, the anti-CSF1R sdAb comprises an amino acid sequence that is at least 96% identical to SEQ ID NO: 16. In some embodiments, the anti-CSF1R sdAb comprises an amino acid sequence that is at least 97% identical to SEQ ID NO: 16. In some embodiments, the anti-CSF1R sdAb comprises an amino acid sequence that is at least 98% identical to SEQ ID NO: 16. In some embodiments, the anti-CSF1R sdAb comprises an amino acid sequence that is at least 99% identical to SEQ ID NO: 16. In some embodiments, the anti-CSF1R sdAb comprises an amino acid sequence of SEQ ID NO: 16.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 17. In some embodiments, the anti-CSF1R sdAb comprises an amino acid sequence that is at least 80% identical to SEQ ID NO: 17. In some embodiments, the anti-CSF1R sdAb comprises an amino acid sequence that is at least 82% identical to SEQ ID NO: 17. In some embodiments, the anti-CSF1R sdAb comprises an amino acid sequence that is at least 85% identical to SEQ ID NO: 17. In some embodiments, the anti-CSF1R sdAb comprises an amino acid sequence that is at least 88% identical to SEQ ID NO: 17. In some embodiments, the anti-CSF1R sdAb comprises an amino acid sequence that is at least 90% identical to SEQ ID NO: 17. In some embodiments, the anti-CSF1R sdAb comprises an amino acid sequence that is at least 91% identical to SEQ ID NO: 17. In some embodiments, the anti-CSF1R sdAb comprises an amino acid sequence that is at least 92% identical to SEQ ID NO: 17. In some embodiments, the anti-CSF1R sdAb comprises an amino acid sequence that is at least 93% identical to SEQ ID NO: 17. In some embodiments, the anti-CSF1R sdAb comprises an amino acid sequence that is at least 94% identical to SEQ ID NO: 17. In some embodiments, the anti-CSF1R sdAb comprises an amino acid sequence that is at least 95% identical to SEQ ID NO: 17. In some embodiments, the anti-CSF1R sdAb comprises an amino acid sequence that is at least 96% identical to SEQ ID NO: 17. In some embodiments, the anti-CSF1R sdAb comprises an amino acid sequence that is at least 97% identical to SEQ ID NO: 17. In some embodiments, the anti-CSF1R sdAb comprises an amino acid sequence that is at least 98% identical to SEQ ID NO: 17. In some embodiments, the anti-CSF1R sdAb comprises an amino acid sequence that is at least 99% identical to SEQ ID NO: 17. In some embodiments, the anti-CSF1R sdAb comprises an amino acid sequence of SEQ ID NO: 17.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 18. In some embodiments, the anti-CSF1R sdAb comprises an amino acid sequence that is at least 80% identical to SEQ ID NO: 18. In some embodiments, the anti-CSF1R sdAb comprises an amino acid sequence that is at least 82% identical to SEQ ID NO: 18. In some embodiments, the anti-CSF1R sdAb comprises an amino acid sequence that is at least 85% identical to SEQ ID NO: 18. In some embodiments, the anti-CSF1R sdAb comprises an amino acid sequence that is at least 88% identical to SEQ ID NO: 18. In some embodiments, the anti-CSF1R sdAb comprises an amino acid sequence that is at least 90% identical to SEQ ID NO: 18. In some embodiments, the anti-CSF1R sdAb comprises an amino acid sequence that is at least 91% identical to SEQ ID NO: 18. In some embodiments, the anti-CSF1R sdAb comprises an amino acid sequence that is at least 92% identical to SEQ ID NO: 18. In some embodiments, the anti-CSF1R sdAb comprises an amino acid sequence that is at least 93% identical to SEQ ID NO: 18. In some embodiments, the anti-CSF1R sdAb comprises an amino acid sequence that is at least 94% identical to SEQ ID NO: 18. In some embodiments, the anti-CSF1R sdAb comprises an amino acid sequence that is at least 95% identical to SEQ ID NO: 18. In some embodiments, the anti-CSF1R sdAb comprises an amino acid sequence that is at least 96% identical to SEQ ID NO: 18. In some embodiments, the anti-CSF1R sdAb comprises an amino acid sequence that is at least 97% identical to SEQ ID NO: 18. In some embodiments, the anti-CSF1R sdAb comprises an amino acid sequence that is at least 98% identical to SEQ ID NO: 18. In some embodiments, the anti-CSF1R sdAb comprises an amino acid sequence that is at least 99% identical to SEQ ID NO: 18. In some embodiments, the anti-CSF1R sdAb comprises an amino acid sequence of SEQ ID NO: 18.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 19. In some embodiments, the anti-CSF1R sdAb comprises an amino acid sequence that is at least 80% identical to SEQ ID NO: 19. In some embodiments, the anti-CSF1R sdAb comprises an amino acid sequence that is at least 82% identical to SEQ ID NO: 19. In some embodiments, the anti-CSF1R sdAb comprises an amino acid sequence that is at least 85% identical to SEQ ID NO: 19. In some embodiments, the anti-CSF1R sdAb comprises an amino acid sequence that is at least 88% identical to SEQ ID NO: 19. In some embodiments, the anti-CSF1R sdAb comprises an amino acid sequence that is at least 90% identical to SEQ ID NO: 19. In some embodiments, the anti-CSF1R sdAb comprises an amino acid sequence that is at least 91% identical to SEQ ID NO: 19. In some embodiments, the anti-CSF1R sdAb comprises an amino acid sequence that is at least 92% identical to SEQ ID NO: 19. In some embodiments, the anti-CSF1R sdAb comprises an amino acid sequence that is at least 93% identical to SEQ ID NO: 19. In some embodiments, the anti-CSF1R sdAb comprises an amino acid sequence that is at least 94% identical to SEQ ID NO: 19. In some embodiments, the anti-CSF1R sdAb comprises an amino acid sequence that is at least 95% identical to SEQ ID NO: 19. In some embodiments, the anti-CSF1R sdAb comprises an amino acid sequence that is at least 96% identical to SEQ ID NO: 19. In some embodiments, the anti-CSF1R sdAb comprises an amino acid sequence that is at least 97% identical to SEQ ID NO: 19. In some embodiments, the anti-CSF1R sdAb comprises an amino acid sequence that is at least 98% identical to SEQ ID NO: 19. In some embodiments, the anti-CSF1R sdAb comprises an amino acid sequence that is at least 99% identical to SEQ ID NO: 19. In some embodiments, the anti-CSF1R sdAb comprises an amino acid sequence of SEQ ID NO: 19.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 20. In some embodiments, the anti-CSF1R sdAb comprises an amino acid sequence that is at least 80% identical to SEQ ID NO: 20. In some embodiments, the anti-CSF1R sdAb comprises an amino acid sequence that is at least 82% identical to SEQ ID NO: 20. In some embodiments, the anti-CSF1R sdAb comprises an amino acid sequence that is at least 85% identical to SEQ ID NO: 1. In some embodiments, the anti-CSF1R sdAb comprises an amino acid sequence that is at least 88% identical to SEQ ID NO: 20. In some embodiments, the anti-CSF1R sdAb comprises an amino acid sequence that is at least 90% identical to SEQ ID NO: 20. In some embodiments, the anti-CSF1R sdAb comprises an amino acid sequence that is at least 91% identical to SEQ ID NO: 20. In some embodiments, the anti-CSF1R sdAb comprises an amino acid sequence that is at least 92% identical to SEQ ID NO: 20. In some embodiments, the anti-CSF1R sdAb comprises an amino acid sequence that is at least 93% identical to SEQ ID NO: 20. In some embodiments, the anti-CSF1R sdAb comprises an amino acid sequence that is at least 94% identical to SEQ ID NO: 20. In some embodiments, the anti-CSF1R sdAb comprises an amino acid sequence that is at least 95% identical to SEQ ID NO: 20. In some embodiments, the anti-CSF1R sdAb comprises an amino acid sequence that is at least 96% identical to SEQ ID NO: 20. In some embodiments, the anti-CSF1R sdAb comprises an amino acid sequence that is at least 97% identical to SEQ ID NO: 20. In some embodiments, the anti-CSF1R sdAb comprises an amino acid sequence that is at least 98% identical to SEQ ID NO: 20. In some embodiments, the anti-CSF1R sdAb comprises an amino acid sequence that is at least 99% identical to SEQ ID NO: 20. In some embodiments, the anti-CSF1R sdAb comprises an amino acid sequence of SEQ ID NO: 20.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 21. In some embodiments, the anti-CSF1R sdAb comprises an amino acid sequence that is at least 80% identical to SEQ ID NO: 21. In some embodiments, the anti-CSF1R sdAb comprises an amino acid sequence that is at least 82% identical to SEQ ID NO: 21. In some embodiments, the anti-CSF1R sdAb comprises an amino acid sequence that is at least 85% identical to SEQ ID NO: 21. In some embodiments, the anti-CSF1R sdAb comprises an amino acid sequence that is at least 88% identical to SEQ ID NO: 21. In some embodiments, the anti-CSF1R sdAb comprises an amino acid sequence that is at least 90% identical to SEQ ID NO: 21. In some embodiments, the anti-CSF1R sdAb comprises an amino acid sequence that is at least 91% identical to SEQ ID NO: 21. In some embodiments, the anti-CSF1R sdAb comprises an amino acid sequence that is at least 92% identical to SEQ ID NO: 21. In some embodiments, the anti-CSF1R sdAb comprises an amino acid sequence that is at least 93% identical to SEQ ID NO: 21. In some embodiments, the anti-CSF1R sdAb comprises an amino acid sequence that is at least 94% identical to SEQ ID NO: 21. In some embodiments, the anti-CSF1R sdAb comprises an amino acid sequence that is at least 95% identical to SEQ ID NO: 21. In some embodiments, the anti-CSF1R sdAb comprises an amino acid sequence that is at least 96% identical to SEQ ID NO: 21. In some embodiments, the anti-CSF1R sdAb comprises an amino acid sequence that is at least 97% identical to SEQ ID NO: 21. In some embodiments, the anti-CSF1R sdAb comprises an amino acid sequence that is at least 98% identical to SEQ ID NO: 21. In some embodiments, the anti-CSF1R sdAb comprises an amino acid sequence that is at least 99% identical to SEQ ID NO: 21. In some embodiments, the anti-CSF1R sdAb comprises an amino acid sequence of SEQ ID NO: 21.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 22. In some embodiments, the anti-CSF1R sdAb comprises an amino acid sequence that is at least 80% identical to SEQ ID NO: 22. In some embodiments, the anti-CSF1R sdAb comprises an amino acid sequence that is at least 82% identical to SEQ ID NO: 22. In some embodiments, the anti-CSF1R sdAb comprises an amino acid sequence that is at least 85% identical to SEQ ID NO: 22. In some embodiments, the anti-CSF1R sdAb comprises an amino acid sequence that is at least 88% identical to SEQ ID NO: 22. In some embodiments, the anti-CSF1R sdAb comprises an amino acid sequence that is at least 90% identical to SEQ ID NO: 22. In some embodiments, the anti-CSF1R sdAb comprises an amino acid sequence that is at least 91% identical to SEQ ID NO: 22. In some embodiments, the anti-CSF1R sdAb comprises an amino acid sequence that is at least 92% identical to SEQ ID NO: 22. In some embodiments, the anti-CSF1R sdAb comprises an amino acid sequence that is at least 93% identical to SEQ ID NO: 22. In some embodiments, the anti-CSF1R sdAb comprises an amino acid sequence that is at least 94% identical to SEQ ID NO: 22. In some embodiments, the anti-CSF1R sdAb comprises an amino acid sequence that is at least 95% identical to SEQ ID NO: 22. In some embodiments, the anti-CSF1R sdAb comprises an amino acid sequence that is at least 96% identical to SEQ ID NO: 22. In some embodiments, the anti-CSF1R sdAb comprises an amino acid sequence that is at least 97% identical to SEQ ID NO: 22. In some embodiments, the anti-CSF1R sdAb comprises an amino acid sequence that is at least 98% identical to SEQ ID NO: 22. In some embodiments, the anti-CSF1R sdAb comprises an amino acid sequence that is at least 99% identical to SEQ ID NO: 22. In some embodiments, the anti-CSF1R sdAb comprises an amino acid sequence of SEQ ID NO: 22.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 23. In some embodiments, the anti-CSF1R sdAb comprises an amino acid sequence that is at least 80% identical to SEQ ID NO: 23. In some embodiments, the anti-CSF1R sdAb comprises an amino acid sequence that is at least 82% identical to SEQ ID NO: 23. In some embodiments, the anti-CSF1R sdAb comprises an amino acid sequence that is at least 85% identical to SEQ ID NO: 23. In some embodiments, the anti-CSF1R sdAb comprises an amino acid sequence that is at least 88% identical to SEQ ID NO: 23. In some embodiments, the anti-CSF1R sdAb comprises an amino acid sequence that is at least 90% identical to SEQ ID NO: 23. In some embodiments, the anti-CSF1R sdAb comprises an amino acid sequence that is at least 91% identical to SEQ ID NO: 23. In some embodiments, the anti-CSF1R sdAb comprises an amino acid sequence that is at least 92% identical to SEQ ID NO: 23. In some embodiments, the anti-CSF1R sdAb comprises an amino acid sequence that is at least 93% identical to SEQ ID NO: 23. In some embodiments, the anti-CSF1R sdAb comprises an amino acid sequence that is at least 94% identical to SEQ ID NO: 23. In some embodiments, the anti-CSF1R sdAb comprises an amino acid sequence that is at least 95% identical to SEQ ID NO: 23. In some embodiments, the anti-CSF1R sdAb comprises an amino acid sequence that is at least 96% identical to SEQ ID NO: 23. In some embodiments, the anti-CSF1R sdAb comprises an amino acid sequence that is at least 97% identical to SEQ ID NO: 23. In some embodiments, the anti-CSF1R sdAb comprises an amino acid sequence that is at least 98% identical to SEQ ID NO: 23. In some embodiments, the anti-CSF1R sdAb comprises an amino acid sequence that is at least 99% identical to SEQ ID NO: 23. In some embodiments, the anti-CSF1R sdAb comprises an amino acid sequence of SEQ ID NO: 23.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 24. In some embodiments, the anti-CSF1R sdAb comprises an amino acid sequence that is at least 80% identical to SEQ ID NO: 24. In some embodiments, the anti-CSF1R sdAb comprises an amino acid sequence that is at least 82% identical to SEQ ID NO: 24. In some embodiments, the anti-CSF1R sdAb comprises an amino acid sequence that is at least 85% identical to SEQ ID NO: 24. In some embodiments, the anti-CSF1R sdAb comprises an amino acid sequence that is at least 88% identical to SEQ ID NO: 24. In some embodiments, the anti-CSF1R sdAb comprises an amino acid sequence that is at least 90% identical to SEQ ID NO: 24. In some embodiments, the anti-CSF1R sdAb comprises an amino acid sequence that is at least 91% identical to SEQ ID NO: 24. In some embodiments, the anti-CSF1R sdAb comprises an amino acid sequence that is at least 92% identical to SEQ ID NO: 24. In some embodiments, the anti-CSF1R sdAb comprises an amino acid sequence that is at least 93% identical to SEQ ID NO: 24. In some embodiments, the anti-CSF1R sdAb comprises an amino acid sequence that is at least 94% identical to SEQ ID NO: 24. In some embodiments, the anti-CSF1R sdAb comprises an amino acid sequence that is at least 95% identical to SEQ ID NO: 24. In some embodiments, the anti-CSF1R sdAb comprises an amino acid sequence that is at least 96% identical to SEQ ID NO: 24. In some embodiments, the anti-CSF1R sdAb comprises an amino acid sequence that is at least 97% identical to SEQ ID NO: 24. In some embodiments, the anti-CSF1R sdAb comprises an amino acid sequence that is at least 98% identical to SEQ ID NO: 24. In some embodiments, the anti-CSF1R sdAb comprises an amino acid sequence that is at least 99% identical to SEQ ID NO: 24. In some embodiments, the anti-CSF1R sdAb comprises an amino acid sequence of SEQ ID NO: 24.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 25. In some embodiments, the anti-CSF1R sdAb comprises an amino acid sequence that is at least 80% identical to SEQ ID NO: 25. In some embodiments, the anti-CSF1R sdAb comprises an amino acid sequence that is at least 82% identical to SEQ ID NO: 25. In some embodiments, the anti-CSF1R sdAb comprises an amino acid sequence that is at least 85% identical to SEQ ID NO: 25. In some embodiments, the anti-CSF1R sdAb comprises an amino acid sequence that is at least 88% identical to SEQ ID NO: 25. In some embodiments, the anti-CSF1R sdAb comprises an amino acid sequence that is at least 90% identical to SEQ ID NO: 25. In some embodiments, the anti-CSF1R sdAb comprises an amino acid sequence that is at least 91% identical to SEQ ID NO: 25. In some embodiments, the anti-CSF1R sdAb comprises an amino acid sequence that is at least 92% identical to SEQ ID NO: 25. In some embodiments, the anti-CSF1R sdAb comprises an amino acid sequence that is at least 93% identical to SEQ ID NO: 25. In some embodiments, the anti-CSF1R sdAb comprises an amino acid sequence that is at least 94% identical to SEQ ID NO: 25. In some embodiments, the anti-CSF1R sdAb comprises an amino acid sequence that is at least 95% identical to SEQ ID NO: 25. In some embodiments, the anti-CSF1R sdAb comprises an amino acid sequence that is at least 96% identical to SEQ ID NO: 25. In some embodiments, the anti-CSF1R sdAb comprises an amino acid sequence that is at least 97% identical to SEQ ID NO: 25. In some embodiments, the anti-CSF1R sdAb comprises an amino acid sequence that is at least 98% identical to SEQ ID NO: 25. In some embodiments, the anti-CSF1R sdAb comprises an amino acid sequence that is at least 99% identical to SEQ ID NO: 25. In some embodiments, the anti-CSF1R sdAb comprises an amino acid sequence of SEQ ID NO: 25.

TABLE B
Exemplary Llama anti-CSF1R VHH Kabat CDR Sequences

| | CDRs | | | | | |
|---|---|---|---|---|---|---|
| Antibody | | SEQ ID | | SEQ ID | | SEQ ID |
| (Ab) | CDR1 | NO | CDR2 | NO | CDR3 | NO |
| Ab1 | YYNMG | 26 | VISQYGSNTYYADSVKG | 51 | LRSGSDWRTIRDYDN | 76 |
| Ab2 | FNVMG | 27 | TIAPGGNTNYFDSARG | 52 | WPFSRDS | 77 |
| Ab3 | SSPMN | 28 | GVTSAGYAYYADWVKG | 53 | GPFHSTAYST | 78 |
| Ab4 | SSLMN | 29 | GVTSGGYSYYAESVKG | 54 | GPFHSIAYAT | 79 |
| Ab5 | STLMN | 30 | GVTSGGYSYYADSVKG | 55 | GPFHSIAYAT | 80 |
| Ab6 | YYNMG | 31 | VISQYGSNTYYADSVKG | 56 | LRSGSDWRTMRDYDN | 81 |
| Ab7 | FNVMG | 32 | TIAPGGKTNYADPAKG | 57 | WPFSRDS | 82 |
| Ab8 | SSPMN | 33 | GVTSAGYAYYADWVKG | 58 | GPFHSTAYST | 83 |
| Ab9 | FNVMG | 34 | TIAPGGKTNYADPAKG | 59 | WPFSRDS | 84 |
| Ab10 | INVMG | 35 | HITSGGRVTYFDSAKG | 60 | LVHRRDS | 85 |
| Ab11 | FNVMG | 36 | TIAPGGNTNYFDSARG | 61 | WPFSRDS | 86 |
| Ab12 | GNVIA | 37 | YITSGGKTLYADSAKG | 62 | LPFKD | 87 |
| Ab13 | FNVMG | 38 | TIAPGGKTNYADPAKG | 63 | WPFSRDS | 88 |
| Ab14 | STVMG | 39 | HITSGGRAQYFDSAKG | 64 | LVHRRDS | 89 |
| Ab15 | STTMN | 40 | GVTSSGYSYYADSVKG | 65 | GPFHSTAYAT | 90 |
| Ab16 | PNVIG | 41 | HITSGGNTLYADSAKG | 66 | LPWTRGS | 91 |
| Ab17 | YNVFG | 42 | HITTGGRTFYSDAGKG | 67 | LPFEAHS | 92 |
| Ab18 | SNVMG | 43 | HITSGGKTQYFDSAKG | 68 | LVHRRDS | 93 |
| Ab19 | FNVMG | 44 | TIAPGGNTNYFDSARG | 69 | WPFSRDS | 94 |
| Ab20 | FNIMA | 45 | RITTGGITWYADPGQG | 70 | LPFRPTY | 95 |
| Ab21 | SNVMG | 46 | HITSDGRTQYFDSAKG | 71 | LVHRRDS | 96 |
| Ab22 | SNVMG | 47 | HITSGGSTQYFDSAKG | 72 | LVHRRDS | 97 |
| Ab23 | FNIMG | 48 | HISSGGNTFYSSSVEG | 73 | LPFRPAS | 98 |
| Ab24 | SSPMN | 49 | GTTSAGYTYYADSVKG | 74 | GPFHSTAYST | 99 |
| Ab25 | GNVMG | 50 | WLTPDGSRTYYADSAKG | 75 | LPFSS | 100 |

In some embodiments, the anti-CSF1R sdAb comprises a complementarity determining region 1 (CDR1) of any one of SEQ ID NOs: 26-50, a CDR2 of any one of SEQ ID NOs: 51-75, and a CDR3 of any one of SEQ ID NOs: 76-100 as shown in Table B.

In some embodiments, the anti-CSF1R sdAb comprises a CDR1 of SEQ ID NO: 26, a CDR2 of SEQ ID NO: 51, and a CDR3 of SEQ ID NO: 76.

In some embodiments, the anti-CSF1R sdAb comprises a CDR1 of SEQ ID NO: 27, a CDR2 of SEQ ID NO: 52, and a CDR3 of SEQ ID NO: 77.

In some embodiments, the anti-CSF1R sdAb comprises a CDR1 of SEQ ID NO: 28, a CDR2 of SEQ ID NO: 53, and a CDR3 of SEQ ID NO: 78.

In some embodiments, the anti-CSF1R sdAb comprises a CDR1 of SEQ ID NO: 29, a CDR2 of SEQ ID NO: 54, and a CDR3 of SEQ ID NO: 79.

In some embodiments, the anti-CSF1R sdAb comprises a CDR1 of SEQ ID NO: 30, a CDR2 of SEQ ID NO: 55, and a CDR3 of SEQ ID NO: 80.

In some embodiments, the anti-CSF1R sdAb comprises a CDR1 of SEQ ID NO: 31, a CDR2 of SEQ ID NO: 56, and a CDR3 of SEQ ID NO: 81.

In some embodiments, the anti-CSF1R sdAb comprises a CDR1 of SEQ ID NO: 32, a CDR2 of SEQ ID NO: 57, and a CDR3 of SEQ ID NO: 82.

In some embodiments, the anti-CSF1R sdAb comprises a CDR1 of SEQ ID NO: 33, a CDR2 of SEQ ID NO: 58, and a CDR3 of SEQ ID NO: 83.

In some embodiments, the anti-CSF1R sdAb comprises a CDR1 of SEQ ID NO: 34, a CDR2 of SEQ ID NO: 59, and a CDR3 of SEQ ID NO: 84.

In some embodiments, the anti-CSF1R sdAb comprises a CDR1 of SEQ ID NO: 35, a CDR2 of SEQ ID NO: 60, and a CDR3 of SEQ ID NO: 85.

In some embodiments, the anti-CSF1R sdAb comprises a CDR1 of SEQ ID NO: 36, a CDR2 of SEQ ID NO: 61, and a CDR3 of SEQ ID NO: 86.

In some embodiments, the anti-CSF1R sdAb comprises a CDR1 of SEQ ID NO: 37, a CDR2 of SEQ ID NO: 62, and a CDR3 of SEQ ID NO: 87.

In some embodiments, the anti-CSF1R sdAb comprises a CDR1 of SEQ ID NO: 38, a CDR2 of SEQ ID NO: 63, and a CDR3 of SEQ ID NO: 88.

In some embodiments, the anti-CSF1R sdAb comprises a CDR1 of SEQ ID NO: 39, a CDR2 of SEQ ID NO: 64, and a CDR3 of SEQ ID NO: 89.

In some embodiments, the anti-CSF1R sdAb comprises a CDR1 of SEQ ID NO: 40, a CDR2 of SEQ ID NO: 65, and a CDR3 of SEQ ID NO: 90.

In some embodiments, the anti-CSF1R sdAb comprises a CDR1 of SEQ ID NO: 41, a CDR2 of SEQ ID NO: 66, and a CDR3 of SEQ ID NO: 91.

In some embodiments, the anti-CSF1R sdAb comprises a CDR1 of SEQ ID NO: 42, a CDR2 of SEQ ID NO: 67, and a CDR3 of SEQ ID NO: 92.

In some embodiments, the anti-CSF1R sdAb comprises a CDR1 of SEQ ID NO: 43, a CDR2 of SEQ ID NO: 68, and a CDR3 of SEQ ID NO: 93.

In some embodiments, the anti-CSF1R sdAb comprises a CDR1 of SEQ ID NO: 44, a CDR2 of SEQ ID NO: 69, and a CDR3 of SEQ ID NO: 94.

In some embodiments, the anti-CSF1R sdAb comprises a CDR1 of SEQ ID NO: 45, a CDR2 of SEQ ID NO: 70, and a CDR3 of SEQ ID NO: 95.

In some embodiments, the anti-CSF1R sdAb comprises a CDR1 of SEQ ID NO: 46, a CDR2 of SEQ ID NO: 71, and a CDR3 of SEQ ID NO: 96.

In some embodiments, the anti-CSF1R sdAb comprises a CDR1 of SEQ ID NO: 47, a CDR2 of SEQ ID NO: 72, and a CDR3 of SEQ ID NO: 97.

In some embodiments, the anti-CSF1R sdAb comprises a CDR1 of SEQ ID NO: 48, a CDR2 of SEQ ID NO: 73, and a CDR3 of SEQ ID NO: 98.

In some embodiments, the anti-CSF1R sdAb comprises a CDR1 of SEQ ID NO: 49, a CDR2 of SEQ ID NO: 74, and a CDR3 of SEQ ID NO: 99.

In some embodiments, the anti-CSF1R sdAb comprises a CDR1 of SEQ ID NO: 50, a CDR2 of SEQ ID NO: 75, and a CDR3 of SEQ ID NO: 100.

TABLE C1
Exemplary Llama Consensus IMGT CDR Sequences

| SEQ | | SEQ ID | | SEQ ID | |
|---|---|---|---|---|---|
| ID NO | CDR1 | NO | CDR2 | NO | CDR3 |
| 147 | GX₁TFSX₂YN | 150 | ISQYGSNT | 153 | TLRSGSDWRTX₃RDYDNW |
| | X₁ is G/L/F | | | | X₃ is I/M |
| | X₂ is S/Y | | | | |
| 148 | GX₄X₅FGSX₆ | 151 | X₇TSX₈GYX₉ | 154 | RGPFHSX₁₀AYX₁₁TR |
| | X₄ is F/M/L | | X₇ is V/T | | X₁₀ is T/I |
| | X₅ is T/A | | X₈ is S/A/G | | X₁₁ is A/S |
| | X₆ is T/S | | X₉ is S/A/T | | |
| 149 | GX₂₁IFX₂₂X₂₃X₂₄V | 152 | IX₂₅X₂₆GGX₂₇T | 155 | X₂₈LPFX₂₉RDS |
| | X₂₁ is DIN | | X₂₅ is T/A/S | | X₂₈ is R/T/S |
| | X₂₂ is R/S | | X₂₆ is S/P/T | | X₂₉ is R/S |
| | X₂₃ is F/S | | X₂₇ is R/N/K | | |
| | X₂₄ is T/N | | | | |

TABLE C2
Exemplary Llama Consensus Kabat CDR Sequences

| SEQ | | SEQ ID | | SEQ ID | |
|---|---|---|---|---|---|
| ID NO | CDR1 | NO | CDR2 | NO | CDR3 |
| 162 | YYNMG | 165 | VISQYGSNTYYADSVKG | 168 | LRSGSDWRTU₁RDYDN |
| | | | | | U₁ is I/M |
| 163 | SU₂U₃MN | 166 | GU₄TSU₅GYURYYAU₇U₈VKG | 169 | GPFHSU₉AYU₁₀T |
| | U₂ is T/S | | U₄ is V/T | | U₉ is T/I |
| | U₃ is T/P/L | | U₅ is S/A/G | | U₁₀ is A/S |
| | | | U₆ is S/A/T | | |
| | | | U₇ is D/E | | |
| | | | U₈ is S/W | | |
| 164 | FNVMG | 167 | TIAPGGU₁₁TNYU₁₂DU₁₃AU₁₄G | 170 | WPFSRDS |
| | | | U₁₁ is N/K | | |
| | | | U₁₂ is F/A | | |
| | | | U₁₃ is S/P | | |
| | | | U₁₄ is R/K | | |

In some embodiments, the anti-CSF1R sdAb comprises a CDR1 comprising a consensus sequence of any one of SEQ ID NOs: 147-149; a CDR2 comprising a consensus sequence of any one of SEQ ID NOs: 150-152; and/or a CDR3 comprising a consensus sequence of any one of SEQ ID NOs: 153-155. In some embodiments, the anti-CSF1R sdAb comprises a CDR1 comprising a consensus sequence of any one of SEQ ID NOs: 147-149; a CDR2 comprising a consensus sequence of any one of SEQ ID NOs: 150-152; and a CDR3 comprising a consensus sequence of any one of SEQ ID NOs: 153-155. In some embodiments, the anti-CSF1R sdAb comprises one or more consensus sequences described in Table C.

In some embodiments, the anti-CSF1R sdAb comprises a consensus CDR1 sequence of SEQ ID NO: 147, wherein X₁ comprises G, L, or F, and wherein X₂ comprises S or Y. In some embodiments, the anti-CSF1R sdAb comprises a CDR2 sequence of SEQ ID NO: 150. In some embodiments, the anti-CSF1R sdAb comprises a consensus CDR3 sequence of SEQ ID NO: 153, wherein X₃ comprises I or M. In some embodiments, the anti-CSF1R sdAb comprises a consensus CDR1 sequence of SEQ ID NO: 147, wherein X₁ comprises G, L, or F, and wherein X₂ comprises S or Y; and a consensus CDR2 sequence of SEQ ID NO: 150; and a consensus CDR3 sequence of SEQ ID NO: 153, wherein X₃ comprises I or M.

In some embodiments, the anti-CSF1R sdAb comprises a consensus CDR1 of SEQ ID NO: 148, wherein X₄ comprises F, M, or L, wherein X₅ comprises T or A, and wherein X₆ comprises T or S. In some embodiments, the anti-CSF1R sdAb comprises a consensus CDR2 sequence of SEQ ID NO: 151, wherein X₇ comprises V or T, wherein X₅ comprises S, A, or G, and wherein X₉ comprises S, A, or T. In some embodiments, the anti-CSF1R sdAb comprises a consensus CDR3 sequence of SEQ ID NO: 154, wherein X₁₀ comprises T or I, and wherein X₁₁ comprises A or S. In some embodiments, the anti-CSF1R sdAb comprises a consensus CDR1 of SEQ ID NO: 148, wherein X₄ comprises F, M, or L, wherein X₅ comprises T or A, and wherein X₆ comprises T or S; a consensus CDR2 sequence of SEQ ID NO: 151, wherein X₇ comprises V or T, wherein X₅ comprises S, A, or G, and wherein X₉ comprises S, A, or T; and a consensus CDR3 sequence of SEQ ID NO: 154, wherein X₁₀ comprises T or I, and wherein X₁₁ comprises A or S.

In some embodiments, the anti-CSF1R sdAb comprises a consensus CDR1 of SEQ ID NO: 149, wherein X₂₁ comprises D or N, wherein X₂₂ comprises R or S, wherein X₂₃ comprises F or S, and wherein X₂₄ comprises T or N. In some embodiments, the anti-CSF1R sdAb comprises a consensus CDR2 sequence of SEQ ID NO: 152, wherein X₂₅ comprises T, A, or S, wherein X₂₆ comprises S, P, or T, and wherein X₂₇ comprises R, N, or K. In some embodiments, the anti-CSF1R sdAb comprises a consensus CDR3 sequence of SEQ ID NO: 155, wherein X₂₈ comprises R, T, or S, and wherein X₂₉ comprises R or S. In some embodiments, the anti-CSF1R sdAb comprises a consensus CDR1 of SEQ ID NO: 149, wherein X₂₁ comprises D or N, wherein X₂₂ comprises R or S, wherein X₂₃ comprises F or S, and wherein X₂₄ comprises T or N; a consensus CDR2 sequence of SEQ ID NO: 152, wherein X₂₅ comprises T, A, or S, wherein X₂₆ comprises S, P, or T, and wherein X₂₇ comprises R, N, or K; and a consensus CDR3 sequence of SEQ ID NO: 155, wherein X₂₈ comprises R, T, or S, and wherein X₂₉ comprises R or S.

In some embodiments, the anti-CSF1R sdAb comprises a CDR1 of SEQ ID NO: 162. In some embodiments, the anti-CSF1R sdAb comprises a CDR2 of SEQ ID NO: 165. In some embodiments, the anti-CSF1R sdAb comprises a consensus CDR3 of SEQ ID NO: 168, wherein U₁ comprises I or M. In some embodiments, the anti-CSF1R sdAb comprises a CDR1 of SEQ ID NO: 162; a CDR2 of SEQ ID NO: 165; and a consensus CDR3 of SEQ ID NO: 168, wherein U₁ comprises I or M.

In some embodiments, the anti-CSF1R sdAb comprises a consensus CDR1 of SEQ ID NO: 163, wherein U₂ comprises T or S, and wherein U₃ comprises T, P, or L. In some embodiments, the anti-CSF1R sdAb comprises a consensus CDR2 of SEQ ID NO: 166, wherein U₄ comprises V or T, wherein U₅ comprises S, A, or G, wherein U₆ comprises S, A, or T, wherein U₇ comprises D or E, and wherein U₈ comprises S or W. In some embodiments, the anti-CSF1R sdAb comprises a consensus CDR3 of SEQ ID NO: 169, wherein U₉ comprises T or I, and wherein U₁₀ comprises A or S. In some embodiments, the anti-CSF1R sdAb comprises a consensus CDR1 of SEQ ID NO: 163, wherein U₂ comprises T or S, and wherein U₃ comprises T, P, or L; a consensus CDR2 of SEQ ID NO: 166, wherein U₄ comprises V or T, wherein U₅ comprises S, A, or G, wherein U₆ comprises S, A, or T, wherein U₇ comprises D or E, and wherein U₈ comprises S or W; and a consensus CDR3 of SEQ ID NO: 169, wherein U₉ comprises T or I, and wherein U₁₀ comprises A or S.

In some embodiments, the anti-CSF1R sdAb comprises a CDR1 of SEQ ID NO: 164. In some embodiments, the anti-CSF1R sdAb comprises a consensus CDR2 of SEQ ID NO: 167, wherein U₁₁ comprises N or K, wherein U₁₂ comprises F or A, wherein U₁₃ comprises S or P, and wherein U₁₄ comprises R or K. In some embodiments, the anti-CSF1R sdAb comprises a CDR3 of SEQ ID NO: 170. In some embodiments, the anti-CSF1R sdAb comprises a CDR1 of SEQ ID NO: 164; a consensus CDR2 of SEQ ID NO: 167, wherein U₁₁ comprises N or K, wherein U₁₂ comprises F or A, wherein U₁₃ comprises S or P, and wherein U₁₄ comprises R or K; and a CDR3 of SEQ ID NO: 170.

TABLE D
Exemplary Humanized anti-CSF1R sdAb Sequences

| | | SEQ ID |
|---|---|---|
| Ab | Full-Length Amino Acid Sequence | NO |
| Ab1-9 | QVQLVESGGGVVQPGRSLRLSCAASGGTFSYYNMGWFRQAPGKGREFVAVISQYGSNTYYADS | 101 |
| | VKGRFTISRDNSKNTLYLQMNSLRAEDTAVYYCATLRSGSDWRTIRDYDNWGQGTQVTVSS | |
| Ab1-12 | EVQLLESGGGLVQPGGSLRLSCAASGGTFSYYNMGWFRQAPGQGREFVAVISQYGSNTYYADS | 102 |
| | VKGRFTISRDNSKNTLYLQMNSLRAEDTAVYYCATLRSGSDWRTIRDYDNWGQGTLVTVSS | |
| Ab1-18 | EVQLQASGGGFVQPGGSLRLSCAASGGTFSYYNMGWFRQAPGKEREFVSVISQYGSNTYYADS | 103 |
| | VKGRFTISRDNSKNTVYLQMNSLRAEDTATYYCATLRSGSDWRTIRDYDNWGQGTQVTVSS | |
| Ab1-20 | EVQLVASGGGFVQPGGSLRLSCAASGGTFSYYNMGWFRQAPGKGREFVSVISQYGSNTYYADS | 104 |
| | VKGRFTISRDNSKNTVYLQMNSLRAEDTATYYCATLRSGSDWRTIRDYDNWGQGTLVTVSS | |
| Ab2-10 | EVQLVESGGGLVQPGGSLRLSCAASGDIFSFNVMGWFRQAPGKERELVSVIAPGGNTYYADSVK | 105 |
| | GRFTISRDNSKNTLYLQMNSLRAEDTAVYYCNRWPFSRDSWGQGTLVTVSS | |

In some embodiments, an anti-CSF1R sdAb is humanized.

In some embodiments, the anti-CSF1R sdAb is at least 80%, 82%, 85%, 88%, 90%, 91%, 92%, 93%, 94%, 95%, 96%, 97%, 98%, or 99% identical to any one of SEQ ID NOs: 101-105. In some embodiments, the anti-CSF1R sdAb comprises an amino acid sequence of any one of SEQ ID NOs: 101-105.

In some embodiments, the anti-CSF1R sdAb is at least 80%, 82%, 85%, 88%, 90%, 91%, 92%, 93%, 94%, 95%, 96%, 97%, 98%, or 99% identical to SEQ ID NO: 101. In some embodiments, the anti-CSF1R sdAb comprises an amino acid sequence that is at least 80% identical to SEQ ID NO: 101. In some embodiments, the anti-CSF1R sdAb comprises an amino acid sequence that is at least 82% identical to SEQ ID NO: 101. In some embodiments, the anti-CSF1R sdAb comprises an amino acid sequence that is at least 85% identical to SEQ ID NO: 101. In some embodiments, the anti-CSF1R sdAb comprises an amino acid sequence that is at least 88% identical to SEQ ID NO: 101. In some embodiments, the anti-CSF1R sdAb comprises an amino acid sequence that is at least 90% identical to SEQ ID NO: 101. In some embodiments, the anti-CSF1R sdAb comprises an amino acid sequence that is at least 91% identical to SEQ ID NO: 101. In some embodiments, the anti-CSF1R sdAb comprises an amino acid sequence that is at least 92% identical to SEQ ID NO: 101. In some embodiments, the anti-CSF1R sdAb comprises an amino acid sequence that is at least 93% identical to SEQ ID NO: 101. In some embodiments, the anti-CSF1R sdAb comprises an amino acid sequence that is at least 94% identical to SEQ ID NO: 101. In some embodiments, the anti-CSF1R sdAb comprises an amino acid sequence that is at least 95% identical to SEQ ID NO: 101. In some embodiments, the anti-CSF1R sdAb comprises an amino acid sequence that is at least 96% identical to SEQ ID NO: 101. In some embodiments, the anti-CSF1R sdAb comprises an amino acid sequence that is at least 97% identical to SEQ ID NO: 101. In some embodiments, the anti-CSF1R sdAb comprises an amino acid sequence that is at least 98% identical to SEQ ID NO: 101. In some embodiments, the anti-CSF1R sdAb comprises an amino acid sequence that is at least 99% identical to SEQ ID NO: 101. In some embodiments, the anti-CSF1R sdAb comprises an amino acid sequence of SEQ ID NO: 101.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 102. In some embodiments, the anti-CSF1R sdAb comprises an amino acid sequence that is at least 80% identical to SEQ ID NO: 102. In some embodiments, the anti-CSF1R sdAb comprises an amino acid sequence that is at least 82% identical to SEQ ID NO: 102. In some embodiments, the anti-CSF1R sdAb comprises an amino acid sequence that is at least 85% identical to SEQ ID NO: 102. In some embodiments, the anti-CSF1R sdAb comprises an amino acid sequence that is at least 88% identical to SEQ ID NO: 102. In some embodiments, the anti-CSF1R sdAb comprises an amino acid sequence that is at least 90% identical to SEQ ID NO: 102. In some embodiments, the anti-CSF1R sdAb comprises an amino acid sequence that is at least 91% identical to SEQ ID NO: 102. In some embodiments, the anti-CSF1R sdAb comprises an amino acid sequence that is at least 92% identical to SEQ ID NO: 102. In some embodiments, the anti-CSF1R sdAb comprises an amino acid sequence that is at least 93% identical to SEQ ID NO: 102. In some embodiments, the anti-CSF1R sdAb comprises an amino acid sequence that is at least 94% identical to SEQ ID NO: 102. In some embodiments, the anti-CSF1R sdAb comprises an amino acid sequence that is at least 95% identical to SEQ ID NO: 102. In some embodiments, the anti-CSF1R sdAb comprises an amino acid sequence that is at least 96% identical to SEQ ID NO: 102. In some embodiments, the anti-CSF1R sdAb comprises an amino acid sequence that is at least 97% identical to SEQ ID NO: 102. In some embodiments, the anti-CSF1R sdAb comprises an amino acid sequence that is at least 98% identical to SEQ ID NO: 102. In some embodiments, the anti-CSF1R sdAb comprises an amino acid sequence that is at least 99% identical to SEQ ID NO: 102. In some embodiments, the anti-CSF1R sdAb comprises an amino acid sequence of SEQ ID NO: 102.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 103. In some embodiments, the anti-CSF1R sdAb comprises an amino acid sequence that is at least 80% identical to SEQ ID NO: 103. In some embodiments, the anti-CSF1R sdAb comprises an amino acid sequence that is at least 82% identical to SEQ ID NO: 103. In some embodiments, the anti-CSF1R sdAb comprises an amino acid sequence that is at least 85% identical to SEQ ID NO: 103. In some embodiments, the anti-CSF1R sdAb comprises an amino acid sequence that is at least 88% identical to SEQ ID NO: 103. In some embodiments, the anti-CSF1R sdAb comprises an amino acid sequence that is at least 90% identical to SEQ ID NO: 103. In some embodiments, the anti-CSF1R sdAb comprises an amino acid sequence that is at least 91% identical to SEQ ID NO: 103. In some embodiments, the anti-CSF1R sdAb comprises an amino acid sequence that is at least 92% identical to SEQ ID NO: 103. In some embodiments, the anti-CSF1R sdAb comprises an amino acid sequence that is at least 93% identical to SEQ ID NO: 103. In some embodiments, the anti-CSF1R sdAb comprises an amino acid sequence that is at least 94% identical to SEQ ID NO: 103. In some embodiments, the anti-CSF1R sdAb comprises an amino acid sequence that is at least 95% identical to SEQ ID NO: 103. In some embodiments, the anti-CSF1R sdAb comprises an amino acid sequence that is at least 96% identical to SEQ ID NO: 103. In some embodiments, the anti-CSF1R sdAb comprises an amino acid sequence that is at least 97% identical to SEQ ID NO: 103. In some embodiments, the anti-CSF1R sdAb comprises an amino acid sequence that is at least 98% identical to SEQ ID NO: 103. In some embodiments, the anti-CSF1R sdAb comprises an amino acid sequence that is at least 99% identical to SEQ ID NO: 103. In some embodiments, the anti-CSF1R sdAb comprises an amino acid sequence of SEQ ID NO: 103.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 104. In some embodiments, the anti-CSF1R sdAb comprises an amino acid sequence that is at least 80% identical to SEQ ID NO: 104. In some embodiments, the anti-CSF1R sdAb comprises an amino acid sequence that is at least 82% identical to SEQ ID NO: 104. In some embodiments, the anti-CSF1R sdAb comprises an amino acid sequence that is at least 85% identical to SEQ ID NO: 104. In some embodiments, the anti-CSF1R sdAb comprises an amino acid sequence that is at least 88% identical to SEQ ID NO: 104. In some embodiments, the anti-CSF1R sdAb comprises an amino acid sequence that is at least 90% identical to SEQ ID NO: 104. In some embodiments, the anti-CSF1R sdAb comprises an amino acid sequence that is at least 91% identical to SEQ ID NO: 104. In some embodiments, the anti-CSF1R sdAb comprises an amino acid sequence that is at least 92% identical to SEQ ID NO: 104. In some embodiments, the anti-CSF1R sdAb comprises an amino acid sequence that is at least 93% identical to SEQ ID NO: 104. In some embodiments, the anti-CSF1R sdAb comprises an amino acid sequence that is at least 94% identical to SEQ ID NO: 104. In some embodiments, the anti-CSF1R sdAb comprises an amino acid sequence that is at least 95% identical to SEQ ID NO: 104. In some embodiments, the anti-CSF1R sdAb comprises an amino acid sequence that is at least 96% identical to SEQ ID NO: 104. In some embodiments, the anti-CSF1R sdAb comprises an amino acid sequence that is at least 97% identical to SEQ ID NO: 104. In some embodiments, the anti-CSF1R sdAb comprises an amino acid sequence that is at least 98% identical to SEQ ID NO: 104. In some embodiments, the anti-CSF1R sdAb comprises an amino acid sequence that is at least 99% identical to SEQ ID NO: 104. In some embodiments, the anti-CSF1R sdAb comprises an amino acid sequence of SEQ ID NO: 104.

In some embodiments, the anti-CSF1R sdAb comprises an amino acid sequence that is at least 80%, 82%, 85%, 88%, 90%, 91%, 92%, 93%, 94%, 95%, 96%, 97%, 98%, or 99% identical to SEQ ID NO: 105. In some embodiments, the anti-CSF1R sdAb comprises an amino acid sequence that is at least 80% identical to SEQ ID NO: 105. In some embodiments, the anti-CSF1R sdAb comprises an amino acid sequence that is at least 82% identical to SEQ ID NO: 105. In some embodiments, the anti-CSF1R sdAb comprises an amino acid sequence that is at least 85% identical to SEQ ID NO: 105. In some embodiments, the anti-CSF1R sdAb comprises an amino acid sequence that is at least 88% identical to SEQ ID NO: 105. In some embodiments, the anti-CSF1R sdAb comprises an amino acid sequence that is at least 90% identical to SEQ ID NO: 105. In some embodiments, the anti-CSF1R sdAb comprises an amino acid sequence that is at least 91% identical to SEQ ID NO: 105. In some embodiments, the anti-CSF1R sdAb comprises an amino acid sequence that is at least 92% identical to SEQ ID NO: 105. In some embodiments, the anti-CSF1R sdAb comprises an amino acid sequence that is at least 93% identical to SEQ ID NO: 105. In some embodiments, the anti-CSF1R sdAb comprises an amino acid sequence that is at least 94% identical to SEQ ID NO: 105. In some embodiments, the anti-CSF1R sdAb comprises an amino acid sequence that is at least 95% identical to SEQ ID NO: 105. In some embodiments, the anti-CSF1R sdAb comprises an amino acid sequence that is at least 96% identical to SEQ ID NO: 105. In some embodiments, the anti-CSF1R sdAb comprises an amino acid sequence that is at least 97% identical to SEQ ID NO: 105. In some embodiments, the anti-CSF1R sdAb comprises an amino acid sequence that is at least 98% identical to SEQ ID NO: 105. In some embodiments, the anti-CSF1R sdAb comprises an amino acid sequence that is at least 99% identical to SEQ ID NO: 105. In some embodiments, the anti-CSF1R sdAb comprises an amino acid sequence of SEQ ID NO: 105.

In some embodiments, the CDR sequences are based on the Chothia convention. In some embodiments, the CDR sequences are based on the IMGT convention. In some embodiments, the CDR sequences are based on the North convention. In some embodiments, the CDR sequences are based on the Kabat convention.

TABLE E
Chothia CDR Sequences of Exemplary anti-CSF1R sdAbs

| | Chothia CDRs | | | | | |
|---|---|---|---|---|---|---|
| Antibody | Chothia | SEQ ID | Chothia | SEQ ID | Chothia | SEQ ID |
| (Ab) | CDR1 | NO | CDR2 | NO | CDR3 | NO |
| Ab1 | GGTFSYY | 106 | SQYGSN | 109 | LRSGSDWRTIRDYDN | 112 |
| Ab1-9 | | | | | | |
| Ab1-12 | | | | | | |
| Ab1-18 | | | | | | |
| Ab1-20 | | | | | | |
| Ab2 | GDIFSFN | 107 | APGGN | 110 | WPFSRDS | 113 |
| Ab2-10 | | | | | | |
| Ab3 | GMAFGSS | 108 | TSAGY | 111 | GPFHSTAYST | 114 |

In some embodiments, the anti-CSF1R sdAb comprises CDRs as shown in Table E, wherein the CDR1 comprises any one of SEQ ID NOs: 106-108, wherein the CDR2 comprises any one of SEQ ID NOs: 109-111, and wherein the CDR3 comprises any one of SEQ ID NOs: 112-114.

In some embodiments, the anti-CSF1R sdAb comprises a CDR1 of SEQ ID NO: 106, a CDR2 of SEQ ID NO: 109, and a CDR3 of SEQ ID NO: 112.

In some embodiments, the anti-CSF1R sdAb comprises a CDR1 comprises SEQ ID NO: 107, a CDR2 of SEQ ID NO: 110, and a CDR3 of SEQ TD NO: 113.

In some embodiments, the anti-CSF1R sdAb comprises a CDR1 of SEQ ID NO: 108, a CDR2 of SEQ ID NO: 111, and a CDR3 of SEQ ID NO: 114.

TABLE F
IMGT CDR Sequences of Exemplary anti-CSF1R sdAbs

| | IMGT CDRs | | | | | |
|---|---|---|---|---|---|---|
| Antibody | IMGT | SEQ ID | IMGT | SEQ ID | IMGT | SEQ ID |
| (Ab) | CDR1 | NO | CDR2 | NO | CDR3 | NO |
| Ab1 | GGTFSYYN | 115 | ISQYGSNT | 118 | ATLRSGSDWRTIRDYDN | 121 |
| Ab1-9 | | | | | | |
| Ab1-12 | | | | | | |
| Ab1-18 | | | | | | |
| Ab1-20 | | | | | | |
| Ab2 | GDIFSFNV | 116 | IAPGGNT | 119 | NRWPFSRDS | 122 |
| Ab2-10 | | | | | | |
| Ab3 | GMAFGSSP | 117 | VTSAGYA | 120 | ARGPFHSTAYST | 123 |

In some embodiments, the anti-CSF1R sdAb comprises CDRs as shown in Table F, wherein the CDR1 comprises any one of SEQ ID NOs: 115-117, wherein the CDR2 comprises any one of SEQ ID NOs: 118-120, and wherein the CDR3 comprises any one of SEQ ID NOs: 121-123.

In some embodiments, the anti-CSF1R sdAb comprises a CDR1 comprises SEQ ID NO: 115, a CDR2 of SEQ ID NO: 118, and a CDR3 of SEQ ID NO: 121.

In some embodiments, the anti-CSF1R sdAb comprises a CDR1 of SEQ ID NO: 115, a CDR2 of SEQ ID NO: 118, and a CDR3 of SEQ ID NO: 121.

In some embodiments, the anti-CSF1R sdAb comprises a CDR1 of SEQ ID NO: 116, a CDR2 of SEQ ID NO: 119, and a CDR3 of SEQ ID NO: 122.

In some embodiments, the anti-CSF1R sdAb comprises a CDR1 of SEQ ID NO: 117, CDR2 of SEQ ID NO: 120, and a CDR3 of SEQ ID NO: 123.

TABLE G
North CDR Sequences of Exemplary anti-CSF1R sdAbs

| | North CDRs | | | | | |
|---|---|---|---|---|---|---|
| Antibody | North | SEQ ID | North | SEQ ID | North | SEQ ID |
| (Ab) | CDR1 | NO | CDR2 | NO | CDR3 | NO |
| Ab1 | AASGGTFSYYNMG | 124 | VISQYGSNTY | 127 | ATLRSGSDWRTIRDYDN | 130 |
| Ab1-9 | | | | | | |
| Ab1-12 | | | | | | |
| Ab1-18 | | | | | | |
| Ab1-20 | | | | | | |
| Ab2 | AASGDIFSFNVMG | 125 | TIAPGGNTN | 156 | NRWPFSRDS | 131 |
| Ab2-10 | AASGDIFSFNVMG | 125 | VIAPGGNTY | 128 | NRWPFSRDS | 131 |
| Ab3 | VASGMAFGSSPMN | 126 | GVTSAGYAY | 129 | ARGPFHSTAYST | 132 |

In some embodiments, the anti-CSF1R sdAb comprises CDRs as shown Table G, wherein the CDR1 comprises any one of SEQ ID NOs: 124-126, wherein the CDR2 comprises any one of SEQ ID NOs: 127-129, and wherein the CDR3 comprises any one of SEQ ID NOs: 130-132.

In some embodiments, the anti-CSF1R sdAb comprises a CDR1 of SEQ ID NO: 124, a CDR2 of SEQ ID NO: 127, and a CDR3 of SEQ ID NO: 130.

In some embodiments, the anti-CSF1R sdAb comprises a CDR1 of SEQ ID NO: 125, a CDR2 of SEQ ID NO: 156, and a CDR3 of SEQ ID NO: 131.

In some embodiments, the anti-CSF1R sdAb comprises a CDR1 of SEQ ID NO: 125, a CDR2 of SEQ ID NO: 128, and a CDR3 of SEQ ID NO: 131.

In some embodiments, the anti-CSF1R sdAb comprises a CDR1 of SEQ ID NO: 126, a CDR2 of SEQ ID NO: 129, and a CDR3 of SEQ ID NO: 132.

TABLE H
Kabat CDR Sequences of Exemplary anti-CSF1R sdAbs

| | Kabat CDRs | | | | | |
|---|---|---|---|---|---|---|
| Antibody | Kabat | SEQ ID | Kabat | SEQ ID | Kabat | SEQ ID |
| (Ab) | CDR1 | NO | CDR2 | NO | CDR3 | NO |
| Ab1 | YYNMG | 133 | VISQYGSNTYYADSVKG | 136 | LRSGSDWRTIRDYDN | 139 |
| Ab1-9 | | | | | | |
| Ab1-12 | | | | | | |
| Ab1-18 | | | | | | |
| Ab1-20 | | | | | | |
| Ab2 | FNVMG | 134 | TIAPGGNTNYFDSARG | 157 | WPFSRDS | 140 |
| Ab2-10 | FNVMG | 134 | VIAPGGNTYYADSVKG | 137 | WPFSRDS | 140 |
| Ab3 | SSPMN | 135 | GVTSAGYAYYADWVKG | 138 | GPFHSTAYST | 141 |

In some embodiments, the anti-CSF1R sdAb comprises CDRs as shown in Table H, wherein the CDR1 comprises any one of SEQ ID NOs: 133-135, wherein the CDR2 comprises any one of SEQ ID NOs: 136-138, and wherein the CDR3 comprises any one of SEQ ID NOs: 139-141.

In some embodiments, the anti-CSF1R sdAb comprises a CDR1 of SEQ ID NO: 133, a CDR2 of SEQ ID NO: 136, and a CDR3 of SEQ ID NO: 139.

In some embodiments, the anti-CSF1R sdAb comprises a CDR1 of SEQ ID NO: 134, a CDR2 of SEQ ID NO: 137, and a CDR3 of SEQ ID NO: 140.

In some embodiments, the anti-CSF1R sdAb comprises a CDR1 of SEQ ID NO: 135, a CDR2 of SEQ ID NO: 138, and a CDR3 of SEQ ID NO: 141.

Epitopes of Anti-CSF1R sdAbs

The exemplary anti-CSF1R sdAbs described herein have properties based on the distinct epitope on CSF1R bound by the anti-CSF1R sdAb. The term “epitope” means the amino acids of a target molecule that are contacted by an anti-CSF1R antibody or sdAb, when the antibody or sdAb is bound to the target molecule. An epitope can be contiguous or non-contiguous (e.g., (i) in a single-chain polypeptide, amino acid residues that are not contiguous to one another in the polypeptide sequence but that within the context of the target molecule are bound by an antibody or sdAb, or (ii) in a multimeric protein comprising two or more individual components (e.g., amino acid residues are present on one or more of the individual components but are still bound by the antibody or sdAb). Epitope determinants can include chemically active surface groupings of molecules such as amino acids, sugar side chains, phosphoryl or sulfonyl groups, and can have specific three dimensional structural characteristics, and/or specific charge characteristics. Generally, antigen binding proteins specific for a particular target molecule will preferentially recognize an epitope on the target molecule in a complex mixture of proteins and/or macromolecules.

Methods of characterizing the epitope bound by an antigen binding protein are well known in the art, including, but not limited to, binning (cross-competition) (Miller et al “Epitope binning of murine monoclonal antibodies by a multiplexed pairing assay” J Immunol Methods (2011) 365, 118-25), peptide mapping (e.g., PEPSPOT™) (Albert et al “The B-cell Epitope of the Monoclonal Anti-Factor VIII Antibody ESH8 Characterized by Peptide Array Analysis” 2008 Thromb. Haemost. 99, 634-7), mutagenesis methods such as chimeras (Song et al “Epitope Mapping of Ibalizumab, a Humanized Anti-CD4 Monoclonal Antibody with Anti-HIV-1 Activity in Infected Patients” J. Virol. (2010) 84, 6935-6942), alanine scanning (Cunningham and Wells “High-resolution epitope mapping of HGH-receptor interactions by alanine-scanning mutagenesis” Science (1989) 244, 1081-1085), arginine scanning (Lim et al “A diversity of antibody epitopes can induce signaling through the erythropoietin receptor” Biochemistry (2010) 49, 3797-3804), HD exchange methods (Coates et al “Epitope mapping by amide hydrogen/deuterium exchange coupled with immobilization of antibody, on-line proteolysis, liquid chromatography and mass spectrometry” Rapid Commun. Mass Spectrom. (2009) 23 639-647), NMR cross saturation methods (Morgan et al “Precise epitope mapping of malaria parasite inhibitory antibodies by TROSY NMR cross-saturation” Biochemistry (2005) 44, 518-23), and crystallography (Gerhardt et al “Structure of IL-17A in complex with a potent, fully human neutralizing antibody” J. Mol. Biol (2009) 394, 905-21). The methods vary in the level of detail they provide as to the amino acids comprising the epitope.

The extracellular domain of CSF1R has 5 immunoglobulin-like domains. The three N-terminal domains (D1-D3) are the ligand-binding domains, and the next two domains (D4-D5) are responsible for dimerization and stabilizing the ligand-receptor complex. In some embodiments, the anti-CSF1R sdAb binds to a domain responsible for dimerization. In some embodiments, the anti-CSF1R sdAb prevents dimerization of CSF1R.

In some embodiments, the anti-CSF1R sdAb described herein binds to D4. In some embodiments, the anti-CSF1R sdAb binds to an amino acid sequence comprising residues 299-399 of CSF1R. In some embodiments, the anti-CSF1R sdAb binds to the extracellular domain of CSF1R but does not bind to residues 1-298 or 400-502 of CSF1R.

In some embodiments, the anti-CSF1R sdAb binds to D5. In some embodiments, the anti-CSF1R sdAb binds to an amino acid sequence comprising residues 402-502 of CSF1R. In some embodiments, the anti-CSF1R sdAb binds to the extracellular domain of CSF1R but does not bind to residues 1-401 of CSF1R.

In some embodiments, the anti-CSF1R sdAb binds to at least one amino acid residue selected from I407, A420, W496, and A497 of human CSF1R (SEQ ID NO: 142), or a combination thereof. In some embodiments, the anti-CSF1R sdAb binds to at least two amino acid residues selected from I407, A420, W496, and A497 of human CSF1R (SEQ ID NO: 142). In some embodiments, the anti-CSF1R sdAb binds to at least three amino acid residues selected from I407, A420, W496, and A497 of human CSF1R (SEQ ID NO: 142). In some embodiments, the anti-CSF1R sdAb binds to amino acid residues I407, A420, W496, and A497 of human CSF1R (SEQ ID NO: 142).

In some embodiments, the anti-CSF1R sdAb binds to at least one amino acid residue selected from I407, A420, W496, A497, and 1499 of human CSF1R (SEQ ID NO: 142), or a combination thereof. In some embodiments, the anti-CSF1R sdAb binds to at least two amino acid residues selected from I407, A420, W496, A497, and 1499 of human CSF1R (SEQ ID NO: 142). In some embodiments, the anti-CSF1R sdAb binds to at least three amino acid residues selected from I407, A420, W496, A497, and 1499 of human CSF1R (SEQ ID NO: 142). In some embodiments, the anti-CSF1R sdAb binds to at least four amino acid residues selected from I407, A420, W496, A497, and 1499 of human CSF1R (SEQ ID NO: 142). In some embodiments, the anti-CSF1R sdAb binds to amino acid residues I407, A420, W496, A497, and 1499 of human CSF1R (SEQ ID NO: 142).

In some embodiments, the anti-CSF1R sdAb binds to amino acid residue I407 of human CSF1R (SEQ ID NO: 142). In some embodiments, the anti-CSF1R sdAb binds to amino acid residue A420 of human CSF1R (SEQ ID NO: 142). In some embodiments, the anti-CSF1R sdAb binds to amino acid residue W496 of human CSF1R (SEQ ID NO: 142). In some embodiments, the anti-CSF1R sdAb binds to amino acid residue A497 of human CSF1R (SEQ ID NO: 142). In some embodiments, the anti-CSF1R sdAb binds to amino acid residue 1499 of human CSF1R (SEQ ID NO: 142).

In some embodiments, the anti-CSF1R sdAb binds to amino acid residues I407 and A420 of human CSF1R (SEQ ID NO: 142). In some embodiments, the anti-CSF1R sdAb binds to amino acid residues I407 and A420, but does not bind to a human CSF1R variant consisting of amino acid substitutions I407T and A420D relative to SEQ ID NO: 142.

In some embodiments, the anti-CSF1R sdAb binds to amino acid residues W496 and A497 of human CSF1R (SEQ ID NO: 142). In some embodiments, the anti-CSF1R sdAb binds to amino acid residues W496 and A497, but does not bind to a human CSF1R variant consisting of amino acid substitutions W496N and A497Y relative to SEQ ID NO: 142.

In some embodiments, the anti-CSF1R sdAb binds to amino acid residues W496 A497, and 1499 of human CSF1R (SEQ ID NO: 142).

In some embodiments, the anti-CSF1R sdAb further binds to at least one amino acid residue selected from P402, E403, V404, S405, V406, W408, L417, A421, S422, H437, T438, D439, R440, K465, T467, G494, S495, F498, and P500 of human CSF1R (SEQ ID NO: 142), or a combination thereof. In some embodiments, the anti-CSF1R sdAb further binds to at least two amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least four amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least six amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least eight amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least ten amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least twelve amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least fourteen amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least sixteen amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to at least eighteen amino acid residues selected from P402, E403, V404, S405, V406, W408, L417, A421, S422, H437, T438, D439, R440, K465, T467, G494, S495, F498, and P500 of human CSF1R (SEQ ID NO: 142). In some embodiments, the anti-CSF1R sdAb further binds to amino acid residues P402, E403, V404, S405, V406, W408, L417, A421, S422, H437, T438, D439, R440, K465, T467, G494, S495, F498, and P500 of human CSF1R (SEQ ID NO: 142).

In some embodiments, the anti-CSF1R sdAb binds to at least one amino acid residue selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142), or a combination thereof. In some embodiments, the anti-CSF1R sdAb binds to at least two amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least four amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least six amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least eight amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least ten amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least twelve amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least fourteen amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least sixteen amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142). In some embodiments, the anti-CSF1R sdAb binds to at least eighteen amino acid residues selected from P402, E403, V404, S405, V406, I407, W408, L417, A420, A421, S422, H437, T438, D439, R440, K465, T467, G494, S495, W496, A497, F498, 1499, and P500 of human CSF1R (SEQ ID NO: 142). In some embodiments, the anti-CSF1R sdAb binds to amino acid residues P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142).

In some embodiments, the anti-CSF1R sdAb binds to human CSF1R. In some embodiments, the anti-CSF1R sdAb binds to cynomolgus monkey CSF1R. In some embodiments, the anti-CSF1R sdAb binds to human CSF1R and cynomolgus monkey CSF1R.

In some embodiments, the anti-CSF1R sdAb does not bind to mouse CSF1R. In some embodiments, the anti-CSF1R sdAb binds to mouse CSF1R with a humanized D4 domain. In some embodiments, the anti-CSF1R sdAb binds to mouse CSF1R with a humanized D5 domain.

In some embodiments, the anti-CSF1R sdAb does not bind to mouse CSF1R but does bind to human CSF1R and mouse CSF1R with a humanized D4 domain. In some embodiments, the anti-CSF1R sdAb does not bind to mouse CSF1R but does bind to cynomolgus monkey CSF1R and mouse CSF1R with a humanized D4 domain. In some embodiments, the anti-CSF1R sdAb does not bind to mouse CSF1R but does bind to human CSF1R, cynomolgus monkey CSF1R and mouse CSF1R with a humanized D4 domain.

In some embodiments, the anti-CSF1R sdAb does not bind to mouse CSF1R but does bind to human CSF1R and mouse CSF1R with a humanized D5 domain. In some embodiments, the anti-CSF1R sdAb does not bind to mouse CSF1R but does bind to cynomolgus monkey CSF1R and mouse CSF1R with a humanized D5 domain. In some embodiments, the anti-CSF1R sdAb does not bind to mouse CSF1R but does bind to human CSF1R, cynomolgus monkey CSF1R and mouse CSF1R with a humanized D5 domain.

In some embodiments, the anti-CSF1R sdAb binds to SEQ ID NO: 142. In some embodiments, the anti-CSF1R sdAb binds to an amino acid that is at least 80% identical to SEQ ID NO: 142. In some embodiments, the anti-CSF1R sdAb binds to an amino acid that is at least 85% identical to SEQ ID NO: 142. In some embodiments, the anti-CSF1R sdAb binds to an amino acid that is at least 90% identical to SEQ ID NO: 142. In some embodiments, the anti-CSF1R sdAb binds to an amino acid that is at least 95% identical to SEQ ID NO: 142. In some embodiments, the anti-CSF1R sdAb binds to an amino acid that is at least 96% identical to SEQ ID NO: 142. In some embodiments, the anti-CSF1R sdAb binds to an amino acid that is at least 97% identical to SEQ ID NO: 142. In some embodiments, the anti-CSF1R sdAb binds to an amino acid that is at least 98% identical to SEQ ID NO: 142. In some embodiments, the anti-CSF1R sdAb binds to an amino acid that is at least 99% identical to SEQ ID NO: 142.

In some embodiments, the anti-CSF1R sdAb binds to SEQ ID NO: 159. In some embodiments, the anti-CSF1R sdAb binds to an amino acid that is at least 80% identical to SEQ ID NO: 159. In some embodiments, the anti-CSF1R sdAb binds to an amino acid that is at least 85% identical to SEQ ID NO: 159. In some embodiments, the anti-CSF1R sdAb binds to an amino acid that is at least 90% identical to SEQ ID NO: 159. In some embodiments, the anti-CSF1R sdAb binds to an amino acid that is at least 95% identical to SEQ ID NO: 159. In some embodiments, the anti-CSF1R sdAb binds to an amino acid that is at least 96% identical to SEQ ID NO: 159. In some embodiments, the anti-CSF1R sdAb binds to an amino acid that is at least 97% identical to SEQ ID NO: 159. In some embodiments, the anti-CSF1R sdAb binds to an amino acid that is at least 98% identical to SEQ ID NO: 159. In some embodiments, the anti-CSF1R sdAb binds to an amino acid that is at least 99% identical to SEQ ID NO: 159.

In some embodiments, the anti-CSF1R sdAb binds to SEQ ID NO: 158. In some embodiments, the anti-CSF1R sdAb binds to an amino acid that is at least 90% identical to SEQ ID NO: 158. In some embodiments, the anti-CSF1R sdAb binds to an amino acid that is at least 95% identical to SEQ ID NO: 158. In some embodiments, the anti-CSF1R sdAb binds to an amino acid that is at least 96% identical to SEQ ID NO: 158. In some embodiments, the anti-CSF1R sdAb binds to an amino acid that is at least 97% identical to SEQ ID NO: 158. In some embodiments, the anti-CSF1R sdAb binds to an amino acid that is at least 98% identical to SEQ ID NO: 158. In some embodiments, the anti-CSF1R sdAb binds to an amino acid that is at least 99% identical to SEQ ID NO: 158.

In some embodiments, the anti-CSF1R sdAb binds to SEQ ID NO: 161. In some embodiments, the anti-CSF1R sdAb binds to an amino acid that is at least 90% identical to SEQ ID NO: 161. In some embodiments, the anti-CSF1R sdAb binds to an amino acid that is at least 95% identical to SEQ ID NO: 161. In some embodiments, the anti-CSF1R sdAb binds to an amino acid that is at least 96% identical to SEQ ID NO: 161. In some embodiments, the anti-CSF1R sdAb binds to an amino acid that is at least 97% identical to SEQ ID NO: 161. In some embodiments, the anti-CSF1R sdAb binds to an amino acid that is at least 98% identical to SEQ ID NO: 161. In some embodiments, the anti-CSF1R sdAb binds to an amino acid that is at least 99% identical to SEQ ID NO: 161.

In some embodiments the anti-CSF1R sdAb does not bind to SEQ ID NO: 160.

TABLE I
Exemplary Sequences of CSFIR

| SEQ | | |
|---|---|---|
| ID NO | Description | Sequence |
| 142 | Full length | MGPGVLLLLLVATAWHGQGIPVIEPSVPELVVKPGATVTLRCVGNGSVEWDGPPSP |
| | human CSF1R | HWTLYSDGSSSILSTNNATFQNTGTYRCTEPGDPLGGSAAIHLYVKDPARPWNVLA |
| | | QEVVVFEDQDALLPCLLTDPVLEAGVSLVRVRGRPLMRHTNYSFSPWHGFTIHRAKF |
| | | IQSQDYQCSALMGGRKVMSISIRLKVQKVIPGPPALTLVPAELVRIRGEAAQIVCSASS |
| | | VDVNFDVFLQHNNTKLAIPQQSDFHNNRYQKVLTLNLDQVDFQHAGNYSCVASNVQ |
| | | GKHSTSMFFRWVESAYLNLSSEQNLIQEVTVGEGLNLKVMVEAYPGLQGFNWTYLG |
| | | PFSDHQPEPKLANATTKDTYRHTFTLSLPRLKPSEAGRYSFLARNPGGWRALTFELT |
| | | LRYPPEVSVIWTFINGSGTLLCAASGYPQPNVTWLQCSGHTDRCDEAQVLQVWDDP |
| | | YPEVLSQEPFHKVTVQSLLTVETLEHNQTYECRAHNSVGSGSWAFIPISAGAHTHPP |
| | | DEFLFTPVWVACMSIMALLLLLLLLLLYKYKQKPKYQVRWKIIESYEGNSYTFIDPTQLP |
| | | YNEKWEFPRNNLQFGKTLGAGAFGKVVEATAFGLGKEDAVLKVAVKMLKSTAHADE |
| | | KEALMSELKIMSHLGQHENIVNLLGACTHGGPVLVITEYCCYGDLLNFLRRKAEAMLG |
| | | PSLSPGQDPEGGVDYKNIHLEKKYVRRDSGFSSQGVDTYVEMRPVSTSSNDSFSEQ |
| | | DLDKEDGRPLELRDLLHFSSQVAQGMAFLASKNCIHRDVAARNVLLTNGHVAKIGDF |
| | | GLARDIMNDSNYIVKGNARLPVKWMAPESIFDCVYTVQSDVWSYGILLWEIFSLGLN |
| | | PYPGILVNSKFYKLVKDGYQMAQPAFAPKNIYSIMQACWALEPTHRPTFQQICSFLQ |
| | | EQAQEDRRERDYTNLPSSSRSGGSGSSSSELEEESSSEHLTCCEQGDIAQPLLQPN |
| | | NYQFC |
| 143 | D1 | PVIEPSVPELWKPGATVTLRCVGNGSVEWDGPPSPHWTLYSDGSSSILSTNNATFQ |
| | Residues 21- | NTGTYRCTEPGDPLGGSAAIHLYVKDP |
| | 104 of hCSF1R | |
| 144 | D2 | PWNVLAQEVVVFEDQDALLPCLLTDPVLEAGVSLVRVRGRPLMRHTNYSFSPWHGF |
| | Residues 107- | TIHRAKFIQSQDYQCSALMGGRKVMSISIRLKVQK |
| | 197 of hCSF1R | |
| 145 | D3 | PALTLVPAELVRIRGEAAQIVCSASSVDVNFDVFLQHNNTKLAIPQQSDFHNNRYQKV |
| | Residues 203- | LTLNLDQVDFQHAGNYSCVASNVQGKHSTS |
| | 290 of hCSF1R | |
| 146 | D4 | AYLNLSSEQNLIQEVTVGEGLNLKVMVEAYPGLQGFNWTYLGPFSDHQPEPKLANA |
| | Residues 299- | TTKDTYRHTFTLSLPRLKPSEAGRYSFLARNPGGWRALTFELTLR |
| | 399 of hCSF1R | |
| 158 | D5 | PEVSVIWTFINGSGTLLCAASGYPQPNVTWLQCSGHTDRCDEAQVLQVWDDPYPEV |
| | Residues 402- | LSQEPFHKVTVQSLLTVETLEHNQTYECRAHNSVGSGSWAFIPIS |
| | 502 of hCSF1R | |
| 159 | hCSF1R | PVIEPSVPELVVKPGATVTLRCVGNGSVEWDGPPSPHWTLYSDGSSSILSTNNATFQ |
| | | NTGTYRCTEPGDPLGGSAAIHLYVKDPARPWNVLAQEVVVFEDQDALLPCLLTDPVL |
| | | EAGVSLVRVRGRPLMRHTNYSFSPWHGFTIHRAKFIQSQDYQCSALMGGRKVMSISI |
| | | RLKVQKVIPGPPALTLVPAELVRIRGEAAQIVCSASSVDVNFDVFLQHNNTKLAIPQQ |
| | | SDFHNNRYQKVLTLNLDQVDFQHAGNYSCVASNVQGKHSTSMFFRVVESAYLNLSS |
| | | EQNLIQEVTVGEGLNLKVMVEAYPGLQGFNWTYLGPFSDHQPEPKLANATTKDTYR |
| | | HTFTLSLPRLKPSEAGRYSFLARNPGGWRALTFELTLRYPPEVSVIWTFINGSGTLLC |
| | | AASGYPQPNVTWLQCSGHTDRCDEAQVLQVWDDPYPEVLSQEPFHKVTVQSLLTV |
| | | ETLEHNQTYECRAHNSVGSGSWAFIPISAGAHTHPPDEFHHHHHHHH |
| 160 | mouseCSF1R | APVIEPSGPELWVEPGETVTLRCVSNGSVEWDGPISPYWTLDPESPGSTLTTRNATF |
| | | KNTGTYRCTELEDPMAGSTTIHLYVKDPAHSWNLLAQEVTVVEGQEAVLPCLITDPA |
| | | LKDSVSLMREGGRQVLRKTVYFFSPWRGFIIRKAKVLDSNTYVCKTMVNGRESTST |
| | | GIWLKVNRVHPEPPQIKLEPSKLVRIRGEAAQIVCSATNAEVGFNVILKRGDTKLEIPL |
| | | NSDFQDNYYKKVRALSLNAVDFQDAGIYSCVASNDVGTRTATMNFQWESAYLNLT |
| | | SEQSLLQEVSVGDSLILTVHADAYPSIQHYNWTYLGPFFEDQRKLEFITQRAIYRYTF |
| | | KLFLNRVKASEAGQYFLMAQNKAGWNNLTFELTLRYPPEVSVTWMPVNGSDVLFCD |
| | | VSGYPQPSVTWMECRGHTDRCDEAQALQVWNDTHPEVLSQKPFDKVIIQSQLPIGT |
| | | LKHNMTYFCKTHNSVGNSSQYFRAVSLGQSKQLPDESHHHHHHHH |
| 161 | mD1D2D3D4hD5 | APVIEPSGPELWVEPGETVTLRCVSNGSVEWDGPISPYWTLDPESPGSTLTTRNATF |
| | (mouse | KNTGTYRCTELEDPMAGSTTIHLYVKDPAHSWNLLAQEVTWEGQEAVLPCLITDPA |
| | CSF1R with a | LKDSVSLMREGGRQVLRKTVYFFSPWRGFIIRKAKVLDSNTYVCKTMVNGRESTST |
| | humanized D5 | GIWLKVNRVHPEPPQIKLEPSKLVRIRGEAAQIVCSATNAEVGFNVILKRGDTKLEIPL |
| | domain) | NSDFQDNYYKKVRALSLNAVDFQDAGIYSCVASNDVGTRTATMNFQWESAYLNLT |
| | | SEQSLLQEVSVGDSLILTVHADAYPSIQHYNWTYLGPFFEDQRKLEFITQRAIYRYTF |
| | | KLFLNRVKASEAGQYFLMAQNKAGWNNLTFELTLRYPPEVSVIWTFINGSGTLLCAA |
| | | SGYPQPNVTWLQCSGHTDRCDEAQVLQVWDDPYPEVLSQEPFHKVTVQSLLTVET |
| | | LEHNQTYECRAHNSVGSGSWAFIPISAGAHTHPPDEFHHHHHHHH |

Antibodies or sdAbs that have an identical epitope or overlapping epitope will often cross-compete for binding to the antigen. Thus, in certain embodiments, a sdAb of the invention cross-competes with any one of Ab1-Ab25. To “cross-compete” or “cross-competition” means the antibodies or sdAbs compete for the same epitope or binding site on a target. Such competition can be determined by an assay in which the reference antigen binding protein (e.g., antibody or sdAb) prevents or inhibits specific binding of a test antibody, and vice versa. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al. (1983) Methods in Enzymology 9:242-253), solid phase direct biotin-avidin EIA (see, e.g., Kirkland et al., (1986) J. Immunol. 137:3614-9), solid phase direct labeled assay, solid phase direct labeled sandwich assay, Luminex (Jia et al. “A novel method of Multiplexed Competitive Antibody Binning for the characterization of monoclonal antibodies” J. Immunological Methods (2004) 288, 91-98) and surface plasmon resonance (Song et al. “Epitope Mapping of Ibalizumab, a Humanized Anti-CD4 Monoclonal Antibody with Anti-HIV-1 Activity in Infected Patients” J. Virol. (2010) 84, 6935-42). Usually, when a competing antibody is present in excess, it will inhibit binding of a reference antigen binding protein to a common antigen by at least 50%, 55%, 60%, 65%, 70%, or 75%. In some instances, binding is inhibited by at least 80%, 85%, 90%, 95%, 96%, 97%, 98%, 99%, or more.

In some embodiments, the anti-CSF1R sdAb competes with a reference sdAb for binding to CSF1R, wherein the reference sdAb comprises a CDR1 comprises an amino acid sequence of GGTFSYY (SEQ ID NO: 106), a CDR2 comprising an amino acid sequence of SQYGSN (SEQ ID NO: 109), and a CDR3 comprising an amino acid sequence of LRSGSDWRTIRDYDN (SEQ ID NO: 112). In some embodiments, the reference sdAb comprises a CDR1 comprising an amino acid sequence of GDIFSFN (SEQ ID NO: 107), a CDR2 comprising an amino acid sequence of APGGN (SEQ ID NO: 110), and a CDR3 comprising an amino acid sequence of WPFSRDS (SEQ ID NO: 113). In some embodiments, the reference sdAb comprises a CDR1 comprising an amino acid sequence of GMAFGSS (SEQ ID NO: 108), a CDR2 comprising an amino acid sequence of TSAGY (SEQ ID NO: 111), and a CDR3 comprising an amino acid sequence of GPFHSTAYST (SEQ ID NO: 114).

In some embodiments, the anti-CSF1R sdAb binds to the same epitope as a reference sdAb, wherein the reference sdAb comprises a CDR1 comprises an amino acid sequence of GGTFSYY (SEQ ID NO: 106), a CDR2 comprising an amino acid sequence of SQYGSN (SEQ ID NO: 109), and a CDR3 comprising an amino acid sequence of LRSGSDWRTIRDYDN (SEQ ID NO: 112). In some embodiments, the reference sdAb comprises a CDR1 comprising an amino acid sequence of GDIFSFN (SEQ ID NO: 107), a CDR2 comprising an amino acid sequence of APGGN (SEQ ID NO: 110), and a CDR3 comprising an amino acid sequence of WPFSRDS (SEQ ID NO: 113). In some embodiments, the reference sdAb comprises a CDR1 comprising an amino acid sequence of GMAFGSS (SEQ ID NO: 108), a CDR2 comprising an amino acid sequence of TSAGY (SEQ ID NO: 111), and a CDR3 comprising an amino acid sequence of GPFHSTAYST (SEQ ID NO: 114).

Potency of Anti-CSF1R sdAbs

The anti-CSF1R sdAbs of the present invention are CSF1R antagonists. In some embodiments, the anti-CSF1R sdAb blocks dimerization of CSF1R. In some embodiments, the anti-CSF1R sdAb prevents dimerization of CSF1R. In some embodiments, the anti-CSF1R sdAb blocks ligand binding. In some embodiments, the anti-CSF1R sdAb prevents ligand binding.

In some embodiments, the anti-CSF1R sdAb inhibits CSF1 activity. In some embodiments, the anti-CSF1R sdAb inhibits IL-34 activity. In some embodiments, the anti-CSF1R sdAb inhibits macrophage proliferation.

In some embodiments, the anti-CSF1R sdAb reduces CSF1 activity. In some embodiments, the anti-CSF1R sdAb reduces IL-34 activity. In some embodiments, the anti-CSF1R sdAb reduces macrophage proliferation.

In some embodiments, the anti-CSF1R sdAb binds to CSF1R with a binding affinity (K_{D}) of, for example, less than 12 nM, less than 11 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.4 nM, less than 0.3 nM, less than 0.2 nM, or less than 0.1 nM.

In some embodiments, the anti-CSF1R sdAb binds to CSF1R with a binding affinity (K_{D}) of, for example, between 0.05 nM and 12 nM, between 0.05 and 0.1 nM, between 0.05 and 0.2 nM, between 0.05 and 1 nM, between 0.05 and 2 nM, between 0.05 and 5 nM, between 0.1 and 0.2 nM, between 0.1 and 1 nM, between 0.1 and 2 nM, between 0.1 and 3 nM, between 0.1 and 4 nM, between 0.1 and 5 nM, between 0.5 and 1 nM, between 0.5 and 2 nM, between 0.5 and 5 nM, between 0.5 and 6 nM, between 0.5 and 7 nM, between 0.5 and 8 nM, between 0.5 and 10 nM, between 0.5 and 12 nM, between 1 and 12 nM, between 1 and 11 nM, between 1 and 10 nM, between 1 and 9 nM, between 1 and 8 nM, between 1 and 7 nM, between 1 and 6 nM, between 1 and 5 nM, between 1 and 4 nM, between 1 and 3 nM, or between 1 and 2 nM.

In some embodiments, the anti-CSF1R sdAb binds to cynomolgus CSF1R with a binding affinity (K_{D}) of, for example less than 7 nM, less than 6 nM, less than 5 nM, less than 4 nM, less than 3 nM, less than 2 nM, less than 1 nM, less than 0.5 nM, less than 0.4 nM, less than 0.3 nM, less than 0.2 nM, less than 0.1 nM, or less than 0.05 nM.

In some embodiments, the anti-CSF1R sdAb binds to cynomolgus CSF1R with a binding affinity (KD) of, for example, between 0.05 nM and 6 nM, between 0.05 nM and 0.1 nM, between 0.05 nM and 0.2 nM, between 0.05 nM and 0.25 nM, between 0.05 nM and 0.5 nM, between 0.05 nM and 0.6 nM, between 0.05 nM and 1 nM, between 0.05 nM and 2 nM, between 0.05 nM and 3 nM, between 0.05 nM and 4 nM, between 0.05 nM and 5 nM, between 0.2 nM and 6 nM, between 0.2 nM and 0.5 nM, between 0.2 nM and 0.6 nM, between 0.2 nM and 1 nM, between 0.2 nM and 2 nM, between 0.2 nM and 3 nM, between 0.2 nM and 4 nM, between 0.2 nM and 5 nM, between 0.5 nM and 6 nM, between 0.5 nM and 1 nM, between 0.5 nM and 2 nM, between 0.5 nM and 3 nM, between 0.5 nM and 4 nM, between 0.5 nM and 5 nM, between 2 and 6 nM, between 2 and 5 nM, between 2 and 4 nM, or between 2 and 3 nM.

In some embodiments, the anti-CSF1R sdAb inhibits CSF1 activity. In some embodiments, the anti-CSF1R sdAb inhibits CSF1 activity as measured by a cellular reporting assay. In some embodiments, the anti-CSF1R sdAb inhibits CSF1 activity as measured by a macrophage proliferation assay. In some embodiments, the anti-CSF1R sdAb inhibits CSF1 activity as measured by a macrophage proliferation assay and a cellular reporter assay.

In some embodiments, the anti-CSF1R sdAb inhibits CSF1 activity with an IC₅₀ value of, for example, less than 0.1 nM, less than 0.3 nM, less than 0.5 nM, less than 1 nM, less than 1.5 nM, less than 2 nM, less than 2.5 nM, less than 3 nM, less than 3.5 nM, or less than 4 nM.

In some embodiments, the anti-CSF1R sdAb inhibits CSF1 activity with an IC₅₀ value of, for example, between 0.1 and 4 nM, between 0.1 and 0.3 nM, between 0.1 and 0.5 nM, between 0.1 and 1 nM, between 0.1 and 1.5 nM, between 0.1 and 2 nM, between 0.1 and 2.5 nM, between 0.1 and 3 nM, between 0.1 and 3.5 nM, between 0.3 and 4 nM, between 0.3 and 0.5 nM, between 0.3 and 1 nM, between 0.3 and 1.5 nM, between 0.3 and 2 nM, between 0.3 and 2.5 nM, between 0.3 and 3 nM, between 0.3 and 3.5 nM, between 0.5 and 4 nM, between 0.5 and 1 nM, between 0.5 and 1.5 nM, between 0.5 and 2 nM, between 0.5 and 2.5 nM, between 0.5 and 3 nM, between 0.5 and 3.5 nM, between 1 and 1.5 nM, between 1 and 2 nM, between 1 and 2.5 nM, between 1 and 3 nM, between 1 and 3.5 nM, or between 1 and 4 nM.

In some embodiments, the anti-CSF1R sdAb inhibits IL-34 activity. In some embodiments, the anti-CSF1R sdAb inhibits IL-34 activity as measured by a cellular reporting assay. In some embodiments, the anti-CSF1R sdAb inhibits IL-34 activity as measured by a macrophage proliferation assay. In some embodiments, the anti-CSF1R sdAb inhibits IL-34 activity as measured by a macrophage proliferation assay and a cellular reporter assay.

In some embodiments, the anti-CSF1R sdAb inhibits IL-34 activity with an IC₅₀ value of, for example, less than 22 nM, less than 20 nM, less than 18 nM, less than 16 nM, less than 14 nM, less than 12 nM, less than 10 nM, less than 9 nM, less than 8 nM, less than 7 nM, less than 6 nM, less than 5 nM, less than 4 nM, less than 3 nM, or less than 2 nM.

In some embodiments, the anti-CSF1R sdAb inhibits IL-34 activity with an IC₅₀ value of, for example, between 2 and 22 nM, between 2 and 20 nM, between 2 and 18 nM, between 2 and 16 nM, between 2 and 14 nM, between 2 and 12 nM, between 2 and 10 nM, between 2 and 9 nM, between 2 and 8 nM, between 2 and 7 nM, between 2 and 6 nM, between 2 and 5 nM, between 2 and 4 nM, between 2 and 3 nM, between 3 and 22 nM, between 3 and 20 nM, between 3 and 18 nM, between 3 and 16 nM, between 3 and 14 nM, between 3 and 12 nM, between 3 and 10 nM, between 3 and 9 nM, between 3 and 8 nM, between 3 and 7 nM, between 3 and 6 nM, between 3 and 5 nM, between 3 and 4 nM, between 4 and 22 nM, between 4 and 20 nM, between 4 and 18 nM, between 4 and 16 nM, between 4 and 14 nM, between 4 and 12 nM, between 4 and 10 nM, between 4 and 9 nM, between 4 and 8 nM, between 4 and 7 nM, between 4 and 6 nM, between 4 and 5 nM, between 5 and 22 nM, between 5 and 20 nM, between 5 and 18 nM, between 5 and 16 nM, between 5 and 14 nM, between 5 and 12 nM, between 5 and 10 nM, between 9 and 22 nM, between 9 and 20 nM, between 9 and 18 nM, between 9 and 16 nM, between 9 and 14 nM, between 9 and 12 nM, between 9 and 10 nM, between 10 and 22 nM, between 10 and 20 nM, between 10 and 18 nM, between 10 and 16 nM, between 10 and 14 nM, between 10 and 12 nM, between 12 and 22 nM, between 12 and 20 nM, between 12 and 18 nM, between 12 and 16 nM, between 12 and 14 nM, between 15 and 22 nM, between 15 and 20 nM, between 15 and 18 nM, between 15 and 16 nM, between 16 and 22 nM, between 16 and 20 nM, between 16 and 18 nM, between 18 and 22 nM, between 18 and 20 nM, or between 20 and 22 nM.

In some embodiments, the IC₅₀ value is determined by cellular reporter assay and macrophage proliferation assay. In some embodiments, the IC₅₀ value is determined by cellular reporter assay or macrophage proliferation assay. In some embodiments, the IC50 value is determined by cellular reporter assay. In some embodiments, the IC50 value is determined by macrophage proliferation assay.

### Half-Life Extension Domain

A sdAb described herein can be fused to any suitable half-life extension domain known in the art.

In some embodiments, an anti-CSF1R sdAb is fused to a half-life extension domain. In some embodiments, a half-life extension domain is an Fc domain. In some embodiments, a half-life extension domain is a peptide that binds human serum albumin (HSA). In some embodiments, a half-life extension domain is an antibody fragment (e.g., Fab, sdAb, VHH) that binds HSA. In some embodiments, the half-life extension domain is HSA.

In some embodiments a sdAb (e.g., an anti-CSF1R sdAb) is fused to an Fc region (sdAb-Fc). In some embodiments, a sdAb (e.g., an anti-CSF1R sdAb) is fused to a CH2 domain. In some embodiments, a sdAb (e.g., an anti-CSF1R sdAb) is fused to a CH3 domain. In some embodiments, a sdAb (e.g., an anti-CSF1R sdAb) is fused to a CH2 and a CH3 domain.

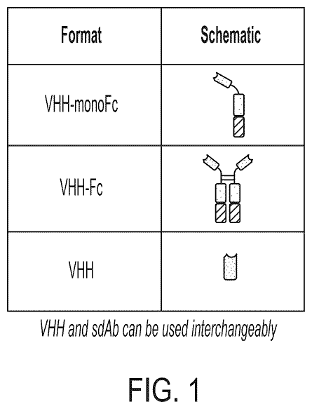

In some embodiments, a sdAb (e.g., an anti-CSF1R sdAb) of the present invention can be used in a format shown in FIG. 1. In some embodiments, a sdAb fused to an Fc region (e.g., an anti-CSF1R sdAb-Fc) is monovalent. In some embodiments, a monovalent sdAb fused to an Fc region comprises one polypeptide. In some embodiments, a sdAb fused to an Fc region (e.g., an anti-CSF1R sdAb-Fc) is bivalent. In some embodiments, a bivalent sdAb fused to an Fc region comprises two polypeptides. In some embodiments, a sdAb (e.g., an anti-CSF1R sdAb) is fused to the N-terminus of an Fc region. In some embodiments, a sdAb (e.g., an anti-CSF1R sdAb) is fused to the C-terminus of an Fc region.

In one aspect, the present invention provides, among other things an anti-CSF1R sdAb-Fc fusion protein, wherein the sdAb comprises any one of amino acid sequences shown in Tables A and D.

In one aspect, the present invention provides, among other things, an anti-CSF1R sdAb-Fc fusion protein, wherein the sdAb comprises a CDR1, a CDR2, and a CDR3 as shown in Tables B, C1, C2, E, F, G, or H.

In some embodiments, an anti-CSF1R sdAb-Fc fusion protein is a homodimer. In some embodiments, an anti-CSF1R sdAb-Fc fusion protein is a heterodimer.

In some embodiments, a sdAb (e.g., an anti-CSF1R sdAb) is fused to a peptide that binds to human serum albumin. In some embodiments, a sdAb (e.g., an anti-CSF1R sdAb) is fused to a sdAb that binds to human serum albumin (e.g., an anti-HSA sdAb). In some embodiments, a first sdAb and a second sdAb that bind to CSF1R (e.g., anti-CSF1R sdAbs) are fused to a sdAb that binds to human serum albumin (e.g., an anti-HSA sdAb).

In some embodiments, an anti-CSF1R sdAb is fused to the N-terminus of an anti-HSA sdAb. In some embodiments, a first and a second anti-CSF1R sdAb are fused to the N-terminus of an anti-HSA sdAb. In some embodiments, an anti-CSF1R sdAb is fused to the C-terminus of an anti-HSA sdAb. In some embodiments, a first and a second anti-CSF1R sdAb are fused to the C-terminus of an anti-HSA sdAb. In some embodiments, a first anti-CSF1R sdAb is fused to the N-terminus of an anti-HSA sdAb, and a second anti-CSF1R sdAb is fused to the C-terminus of the anti-HSA sdAb. In some embodiments, the first anti-CSF1R sdAb comprises the same amino acid sequence as the second anti-CSF1R sdAb. In some embodiments, the first anti-CSF1R sdAb comprises a different amino acid sequence from the second anti-CSF1R sdAb. Constant Region (Fc)

The Fc region of an antibody interacts with a number of Fc receptors and ligands, imparting an array of important functional capabilities referred to as effector functions. For IgG, the Fc region comprises Ig domains C72 and C73 and the N-terminal hinge leading into Cy2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, incorporated by reference). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γδ cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290, incorporated by reference). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). A number of structures have been solved of the extracellular domains of human FcγRs, including FcγRIIa (pdb accession code 1H9V) (Sondermann et al., 2001, J Mol Biol 309:737-749) (pdb accession code 1 FCG) (Maxwell et al., 1999, Nat Struct Biol 6:437-442), FcγRIIb (pdb accession code 2FCB) (Sondermann et al., 1999, Embo J 18:1095-1103); and FcγRIIIb (pdb accession code 1E4J) (Sondermann et al., 2000, Nature 406:267-273, incorporated by reference). All FcγRs bind the same region on Fc, at the N-terminal end of the C72 domain and the preceding hinge. This interaction is well characterized structurally (Sondermann et al., 2001, J Mol Biol 309:737-749 incorporated by reference), and several structures of the human Fc bound to the extracellular domain of human FcγRIIIb have been solved (pdb accession code 1E4K)(Sondermann et al., 2000, Nature 406:267-273) (pdb accession codes 1IIS and 1IIX) (Radaev et al., 2001, J Biol Chem 276:16469-16477, incorporated by reference), as well as has the structure of the human IgE Fd/FcεRIa complex (pdb accession code 1F6A) (Garman et al., 2000, Nature 406:259-266, incorporated by reference).

An overlapping but separate site on Fc serves as the interface for the complement protein C1q. In the same way that Fc/FcγR binding mediates ADCC, Fc/C1q binding mediates complement dependent cytotoxicity (CDC). C1q forms a complex with the serine proteases C1r and Cis to form the C1 complex. C1q is capable of binding six antibodies, although binding to two IgGs is sufficient to activate the complement cascade. Similar to Fc interaction with FcγRs, different IgG subclasses have different affinity for C1q, with IgG1 and IgG3 typically binding substantially better to the FcγRs than IgG2 and IgG4.

A site on Fc between the Cγ2 and Cγ3 domains mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766, incorporated by reference). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Available structures of the rat Fc/FcRn complex (Martin et al., 2001, Mol Cell 7:867-877, incorporated by reference), and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481, incorporated by reference) provide insight into the interaction of Fc with these proteins.

In some embodiments, an antibody or fragments thereof comprises an optimized Fc variants useful in a variety of contexts. As outlined above, current antibody therapies suffer from a variety of problems. The present invention provides a promising means for enhancing the therapeutic efficacy of antibodies is via abolishment of their ability to mediate cytotoxic effector functions such as ADCC, ADCP, and CDC.

### Fc Variants

An Fc polypeptide that comprises an Fc variant described herein is referred to as an “Fc polypeptide”. Fc polypeptides of the present invention include polypeptides that comprise the Fc variants in the context of a larger polypeptide, such as an antibody or Fc fusion. That is, Fc polypeptides include antibodies and Fc fusions that comprise Fc variants of the present invention. Fc polypeptides of the present invention also include polypeptides that comprise little or no additional polypeptide sequence other than the Fc region, referred to as an isolated Fc. Fc polypeptides described in the present invention also include fragments of the Fc region. As described below, any of the aforementioned Fc polypeptides may be fused to one or more fusion partners or conjugate partners to provide desired functional properties.

The parent Fc polypeptides described herein may be derived from a wide range of sources, and may be substantially encoded by one or more Fc genes from any organism, including but not limited to humans, rodents including but not limited to mice and rats, lagomorpha such as rabbits and hares, camelidae such as camels, llamas, and dromedaries, and non-human primates, including but not limited to Prosimians, Platyrrhini (New World monkeys), Cercopithecoidea (Old World monkeys), and Hominoidea include the Gibbons, Lesser and Great Apes, with humans most preferred. The parent Fc polypeptides of the present invention may be substantially encoded by immunoglobulin genes belonging to any of the antibody classes, including but not limited to sequences belonging to the IgG (including human subclasses IgG1, IgG2, IgG3, or IgG4), IgA (including human subclasses IgA1 and IgA2), IgD, IgE, IgG, or IgM classes of antibodies. The parent Fc polypeptides of the present invention comprise sequences belonging to the human IgG class of antibodies. For example, the parent Fc polypeptide may be a parent antibody, for example a human IgG1 antibody, a human IgA antibody, or a mouse IgG2a or IgG2b antibody. Said parent antibody may be nonhuman, chimeric, humanized, or fully human as described in detail below. The parent Fc polypeptide may be modified or engineered in some way, for example a parent antibody may be affinity matured, or may possess engineered glycoforms, all as described more fully below. Alternatively, the parent Fc polypeptide may be an Fc fusion, for example an Fc fusion wherein the fusion partner targets a cell surface receptor. Alternatively, the parent Fc polypeptide may be an isolated Fc region, comprising little or no other polypeptide sequence outside the Fc region. The parent Fc polypeptide may be a naturally existing Fc region, or may be an existing engineered variant of an Fc polypeptide. What is important is that the parent Fc polypeptide comprise an Fc region, which can then be mutated to generate an Fc variant.

### Optimized Properties

Fc variants described herein are optimized for a number of therapeutically relevant properties. An Fc variant comprises one or more amino acid modifications relative to a parent Fc polypeptide, wherein said amino acid modification(s) provide one or more optimized properties. An Fc variant of the present invention differs in amino acid sequence from its parent Fc polypeptide by virtue of at least one amino acid modification. In some embodiments, Fc variants have at least one amino acid modification compared to the parent. Alternatively, in some embodiments the Fc variants may have more than one amino acid modification as compared to the parent, for example from about one to fifty amino acid modifications, from about one to ten amino acid modifications, or from about one to about five amino acid modifications compared to the parent. Thus the sequences of the Fc variants and those of the parent Fc polypeptide are substantially homologous. For example, the variant Fc variant sequences herein will possess about 80% homology with the parent Fc variant sequence, preferably at least about 90% homology, and most preferably at least about 95% homology.

The Fc variants may be optimized for a variety of properties. An Fc variant that is engineered or predicted to display one or more optimized properties is herein referred to as an “optimized Fc variant”. Properties that may be optimized include but are not limited to enhanced or reduced affinity for an FcγR. In some embodiments, the Fc variants are optimized to have reduced or ablated affinity for a human FcγR, including but not limited to FcγRI, FcγRIIa, FcγRIIb, FcγRIIc, FcγRIIIa, and FcγRIIIb. These embodiments are anticipated to provide Fc polypeptides with enhanced therapeutic properties in humans, for example reduced effector function and reduced toxicity. In other embodiments, Fc variants provide enhanced affinity for one or more FcγRs, yet reduced affinity for one or more other FcγRs. For example, an Fc variant may have enhanced binding to FcγRIIIa, yet reduced binding to FcγRIIb. Alternately, an Fc variant may have enhanced binding to FcγRIIa and FcγRI, yet reduced binding to FcγRIIb. In yet another embodiment, an Fc variant may have enhanced affinity for FcγRIIb, yet reduced affinity to one or more activating FcγRs.

In some embodiments, an Fc variant has reduced or ablated affinity for FcγRI. In some embodiments, an Fc variant has reduced or ablated affinity for FcγRIIa. In some embodiments, an Fc variant has reduced or ablated affinity for FcγRIIb. In some embodiments, an Fc variant has reduced or ablated affinity for FcγRIIc. In some embodiments, an Fc variant has reduced or ablated affinity for FcγRIIIa. In some embodiments, an Fc variant has reduced or ablated affinity for FcγRIIIb. In some embodiments, an Fc variant has reduced or ablated affinity for C1q. In some embodiments, an Fc variant has enhanced affinity for FcRn. In some embodiments, an Fc variant maintains affinity for FcRn. In some embodiments, an Fc variant has reduced or ablated affinity for FcγRI, FcγRIIa, FcγRIIb, FcγRIIIa, FcγRIIIb, and C1q. In some embodiments, an Fc variant has reduced or ablated affinity for FcγRI, FcγRIIa, FcγRIIb, FcγRIIIa, FcγRIIIb, and C1q, and retains binding to FcRn.

The Fc variants may also be optimized for enhanced functionality and/or solution properties in aglycosylated form. In a preferred embodiment, the aglycosylated Fc variants bind an Fc ligand with reduced affinity than the aglycosylated form of the parent Fc variant. Said Fc ligands include but are not limited to FcγRs, C1q, FcRn, and proteins A and G, and may be from any source including but not limited to human, mouse, rat, rabbit, or monkey, preferably human. In an alternately preferred embodiment, the Fc variants are optimized to be more stable and/or more soluble than the aglycosylated form of the parent Fc variant.

### Other Antibody Formats

The CDRs and the sdAb sequences disclosed herein can be adapted to various antibody formats known in the art, including but not limited to a traditional antibody, Fab, scFv, diabody, minibody, and scFab. In some embodiments, a Fab is fused to an Fc region. In some embodiments, scFv is fused to an Fc region. In some embodiments, a diabody is fused to an Fc region. In some embodiments, a minibody is fused to an Fc region. In some embodiments, an scFab is fused to an Fc region.

A traditional antibody, also known as an immunoglobulin, is a Y-shaped structure which consists of four polypeptides two heavy chains and two light chains. This structure allows antibody molecules to carry out their dual functions: antigen binding and biological activity mediation. Each function is carried out by different parts of the antibody: fragment antigen-binding (Fab fragment) and fragment crystallizable region (Fc region). Fab fragment is a region on an antibody that binds to antigens. It is composed of one constant and one variable domain of each of the heavy and the light chain. These domains shape the paratope—the antigen-binding site—at the amino terminal end of the monomer. Fc region is the tail region of an antibody that interacts with cell surface receptors called Fc receptors and some proteins of the complement system. This property allows antibodies to activate the immune system. The Fc regions of immunoglobulin Gs bear a highly conserved N-glycosylation site.

Within the VH and VL regions exists three CDRs, hypervariable amino acid sequences, forming the antigen-binding sites of each Fab. The interface of each heavy and light chain CDRs with the constant segments creates a three-dimensional binding structure with high specificity for a distinct antigenic determinant or epitope.

Nucleic Acid Encoding Anti-CSF1R sdAbs

In some embodiments, the present invention provides, among other things, a nucleic acid encoding an anti-CSF1R sdAb described herein. In some embodiments, a nucleic acid encodes CDRs of the anti-CSF1R sdAb. In some embodiments, a nucleic acid encodes fragments of the anti-CSF1R sdAb. In some embodiments, a nucleic acid encodes an anti-CSF1R sdAb described herein. In some embodiments, a nucleic acid encodes the anti-CSF1R sdAb fused to an Fc region. In some embodiments, a nucleic acid encodes an anti-CSF1R VHH described herein. In some embodiments, a nucleic acid encodes the anti-CSF1R VHH fused to an Fc region.

In some embodiments, a nucleic acid is a DNA. In some embodiments, a nucleic acid is a cDNA. In some embodiments, a nucleic acid is an RNA. In some embodiments, a nucleic acid is a messenger RNA (mRNA).

In some embodiments, the present invention provides, among other things, a DNA encoding an anti-CSF1R sdAb described herein. In some embodiments, a DNA encodes CDRs of the anti-CSF1R sdAb. In some embodiments, a DNA encodes fragments of the anti-CSF1R sdAb. In some embodiments, a DNA encodes an anti-CSF1R sdAb described herein. In some embodiments, a DNA encodes the anti-CSF1R sdAb fused to an Fc region. In some embodiments, a DNA encodes an anti-CSF1R VHH described herein. In some embodiments, a DNA encodes the anti-CSF1R VHH fused to an Fc region.

In some embodiments, the present invention provides, among other things, an RNA encoding an anti-CSF1R sdAb described herein. In some embodiments, an RNA encodes CDRs of the anti-CSF1R sdAb. In some embodiments, an RNA encodes fragments of the anti-CSF1R sdAb. In some embodiments, an RNA encodes an anti-CSF1R sdAb described herein. In some embodiments, an RNA encodes the anti-CSF1R sdAb fused to an Fc region. In some embodiments, an RNA encodes an anti-CSF1R VHH described herein. In some embodiments, an RNA encodes the anti-CSF1R VHH fused to an Fc region.

In some embodiments, the RNA is messenger RNA. In some embodiments, the present invention provides, among other things, a messenger RNA encoding an anti-CSF1R sdAb described herein. In some embodiments, a messenger RNA encodes CDRs of the anti-CSF1R sdAb. In some embodiments, a messenger RNA encodes fragments of the anti-CSF1R sdAb. In some embodiments, a messenger RNA encodes an anti-CSF1R sdAb described herein. In some embodiments, a messenger RNA encodes the anti-CSF1R sdAb fused to an Fc region. In some embodiments, a messenger RNA encodes an anti-CSF1R VHH described herein. In some embodiments, a messenger RNA encodes the anti-CSF1R VHH fused to an Fc region.

### Delivery Vehicles

In some embodiments, the present invention provides, among other things, an anti-CSF1R sdAb delivered via a delivery vehicle. As used herein, the terms “delivery vehicle,” “transfer vehicle,” “nanoparticle” or grammatical equivalent, are used interchangeably. In some embodiments, the anti-CSF1R sdAb is delivered unpackaged.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

In some embodiments, the anti-CSF1R sdAb described herein is delivered via a delivery vehicle. In some embodiments, the anti-CSF1R sdAb is delivered as a sdAb via a delivery vehicle. In some embodiments, the anti-CSF1R sdAb is delivered as a sdAb via a delivery vehicle. In some embodiments, the anti-CSF1R sdAb is delivered as a VHH via a delivery vehicle. In some embodiments, the anti-CSF1R sdAb is delivered as a sdAb fused to an Fc region via a delivery vehicle. In some embodiments, the anti-CSF1R sdAb is delivered as a VHH fused to an Fc region via a delivery vehicle. In some embodiments, a nucleic acid encoding the anti-CSF1R sdAb is delivered via a delivery vehicle.

In some embodiments, the anti-CSF1R sdAb described herein is delivered unpackaged. In some embodiments, the anti-CSF1R sdAb is delivered as a sdAb unpackaged. In some embodiments, the anti-CSF1R sdAb is delivered as a sdAb unpackaged. In some embodiments, the anti-CSF1R sdAb is delivered as a VHH unpackaged. In some embodiments, the anti-CSF1R sdAb is delivered as a sdAb fused to an Fc region unpackaged. In some embodiments, the anti-CSF1R sdAb is delivered as a VHH fused to an Fc region unpackaged. In some embodiments, a nucleic acid encoding the anti-CSF1R sdAb is delivered unpackaged.

### Vectors

In some embodiments, the present invention provides, among other things, a vector comprising the nucleic acid encoding the anti-CSF1R sdAb described herein. A vector can further comprise additional sequences such as, for example and without limitation, replication origins, promoters, and genes encoding antibiotic resistant. In some embodiments, the vector is circular. In some embodiments, the vector is linear.

In some embodiments, the vector is a viral vector. In some embodiments, the vector is derived from a lentivirus vector. In some embodiments, the vector is derived from a herpes simplex virus vector. In some embodiments, the vector is derived from a retrovirus vector. In some embodiments, the vector is derived from an adenovirus vector. In some embodiments, the vector is derived from a poxvirus vector. In some embodiments, the vector is derived from a vaccinia virus vector.

In some embodiments, the vector is an adeno-associated virus (AAV) vector. Different serotypes of AAV vectors have been identified with specific tropism and preferential transduction into different cell types.

In some embodiments, the vector is derived from AAV1. In some embodiments, the vector is derived from AAV2. In some embodiments, the vector is derived from AAV3. In some embodiments, the vector is derived from AAV4. In some embodiments, the vector is derived from AAV5. In some embodiments, the vector is derived from AAV6. In some embodiments, the vector is derived from AAV7. In some embodiments, the vector is derived from AAV8. In some embodiments, the vector is derived from AAV9. In some embodiments, the vector is derived from AAV10. In some embodiments, the vector is derived from AAV11. In some embodiments, the vector is derived from AAV12. In some embodiments, the vector is derived from AAV13.

In some embodiments, the anti-CSF1R sdAb described herein is delivered via a vector. In some embodiments, the anti-CSF1R sdAb fused to an Fc region is delivered via a vector. In some embodiments, a nucleic acid encoding the anti-CSF1R sdAb is delivered via a vector. In some embodiments, a nucleic acid encoding the anti-CSF1R sdAb fused to an Fc region is delivered via a vector.

In some embodiments, the anti-CSF1R sdAb described herein is delivered via a viral vector. In some embodiments, the anti-CSF1R sdAb fused to an Fc region is delivered via a viral vector. In some embodiments, a nucleic acid encoding the anti-CSF1R sdAb is delivered via a viral vector. In some embodiments, a nucleic acid encoding the anti-CSF1R sdAb fused to an Fc region is delivered via a viral vector.

In some embodiments, the anti-CSF1R sdAb described herein is delivered via an AAV vector. In some embodiments, the anti-CSF1R sdAb fused to an Fc region is delivered via an AAV vector. In some embodiments, a nucleic acid encoding the anti-CSF1R sdAb is delivered via an AAV vector. In some embodiments, a nucleic acid encoding the anti-CSF1R sdAb fused to an Fc region is delivered via an AAV vector.

### Liposomal Delivery Vehicles

In some embodiments, a suitable delivery vehicle is a liposomal delivery vehicle, e.g., a lipid nanoparticle (LNP). As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid to a target cell or tissue. In some embodiments, a nanoparticle delivery vehicle is a liposome. In some embodiments, a liposome comprises one or more cationic lipids, one or more non-cationic lipids, one or more cholesterol-based lipids and one or more PEG-modified lipids.

In some embodiments, the anti-CSF1R sdAb described herein is delivered via a liposomal delivery vehicle. In some embodiments, the anti-CSF1R sdAb fused to an Fc region is delivered via a liposomal delivery vehicle. In some embodiments, a nucleic acid encoding the anti-CSF1R sdAb is delivered via a liposomal delivery vehicle. In some embodiments, a nucleic acid encoding the anti-CSF1R sdAb fused to a Fc region is delivered via a liposomal delivery vehicle.

In some embodiments, the anti-CSF1R sdAb described herein is delivered via a lipid nanoparticle. In some embodiments, the anti-CSF1R sdAb fused to an Fc region is delivered via a lipid nanoparticle. In some embodiments, a nucleic acid encoding the anti-CSF1R sdAb is delivered via a lipid nanoparticle. In some embodiments, a nucleic acid encoding the anti-CSF1R sdAb fused to a Fc region is delivered via a lipid nanoparticle.

### Therapeutic Use

In some embodiments, the present invention provides, among other things, a method of treating CSF1R mediated disorders (e.g., AMD) by administering an anti-CSF1R sdAb. In some embodiments, the present invention provides, among other things, a method of treating CSF1R mediated disorder in a subject comprising administering to the subject an anti-CSF1R sdAb at a therapeutically effective dose and an administration interval for a treatment period sufficient to improve, stabilize or reduce one or more symptoms of the said disorder. In some embodiments, the present invention provides, among other things, a method of treating CSF1R mediated disorder in a subject comprising administering to the subject a composition comprising a nucleic acid encoding an anti-CSF1R sdAb at a therapeutically effective dose and an administration interval for a treatment period sufficient to improve, stabilize or reduce one or more symptoms of the said disorder. In some embodiments, the present invention provides, among other things, a method of treating CSF1R mediated disorder in a subject comprising administering to the subject a composition comprising mRNA encoding an anti-CSF1R sdAb at a therapeutically effective dose and an administration interval for a treatment period sufficient to improve, stabilize or reduce one or more symptoms of the said disorder. As used herein, a CSF1R mediated disorder is also referred to as a “disease or disorder associated with CSF1R.”

In some embodiments, the CSF1R mediated disorder is Age-related macular degeneration (AMD), Stargardt disease, Retinitis pigmentosa, Central retinal vein occlusion (CRVO), Choroidal neovascularization (CNV), Diabetic retinopathy, Proliferative diabetic retinopathy (PDR), Proliferative vitreoretinopathy, Retinal edema, Retinal vein occlusion (RVO), Retinopathy of prematurity (ROP), Sequela associated with retinal ischemia, Anterior segment neovascularization, Corneal neovascularization, Posterior segment neovascularization, Polypoidal choroidal vasculopathy, Diabetic macular edema (DME), Noninfectious uveitis (NIU), Uveitis, Dry eye syndrome, or Glaucoma.

In some embodiments, the CSF1R mediated disorder is AMD. In some embodiments, the CSF1R mediated disorder is wet AMD. In some embodiments, the CSF1R mediated disorder is dry AMD.

In some embodiments, the CSF1R mediated disorder is Stargardt disease. In some embodiments, the CSF1R mediated disorder is Retinitis pigmentosa. In some embodiments, the CSF1R mediated disorder is CRVO. In some embodiments, the CSF1R mediated disorder is CNV. In some embodiments, the CSF1R mediated disorder is Diabetic retinopathy. In some embodiments, the CSF1R mediated disorder is PDR. In some embodiments, the CSF1R mediated disorder is Proliferative vitreoretinopathy. In some embodiments, the CSF1R mediated disorder is Retinal edema. In some embodiments, the CSF1R mediated disorder is RVO. In some embodiments, the CSF1R mediated disorder is ROP. In some embodiments, the CSF1R mediated disorder is Sequela associated with retinal ischemia. In some embodiments, the CSF1R mediated disorder is Anterior segment neovascularization. In some embodiments, the CSF1R mediated disorder is Corneal neovascularization. In some embodiments, the CSF1R mediated disorder is Posterior segment neovascularization. In some embodiments, the CSF1R mediated disorder is Polypoidal choroidal vasculopathy. In some embodiments, the CSF1R mediated disorder is DME. In some embodiments, the CSF1R mediated disorder is NIU. In some embodiments, the CSF1R mediated disorder is Uveitis. In some embodiments, the CSF1R mediated disorder is Dry eye syndrome. In some embodiments, the CSF1R mediated disorder is Glaucoma.

### Gene Therapy

In one aspect, the present invention, among other things, provide a method of inducing anti-CSF1R sdAb expression in vivo by administration of nucleic acids encoding an anti-CSF1R sdAb, or by administration of an anti-CSF1R sdAb. In some embodiments, a composition comprises nucleic acids encapsulated or complexed with a delivery vehicle. In some embodiments, the delivery vehicle is AAV, liposomes, lipid nanoparticles, solid-lipid nanoparticles, polymers, viruses, sol-gels, or nanogels. In some embodiments, nucleic acids encoding an anti-CSF1R sdAb are packaged in a viral particle.

In some embodiments, a pharmaceutical composition comprising nucleic acids encoding an anti-CSF1R sdAb is used to treat subjects in need thereof. In some embodiments, a pharmaceutical composition comprising a rAAV vector described herein is used to treat subjects in need thereof. The pharmaceutical composition containing a rAAV vector or particle of the invention contains a pharmaceutically acceptable excipient, diluent or carrier. Examples of suitable pharmaceutical carriers are well known in the art and include phosphate buffered saline solutions, water, emulsions, such as oil/water emulsions, various types of wetting agents, sterile solutions and the like. The pharmaceutical composition can be in a lyophilized form. Such carriers can be formulated by conventional methods and are administered to the subject at a therapeutically effective amount.

The rAAV vector is administered to a subject in need thereof via a suitable route. In some embodiments, the rAAV vector is administered by intravenous, intraperitoneal, subcutaneous, or intradermal routes. In one embodiment, the rAAV vector is administered intravenously. In embodiments, the intradermal administration comprises administration by use of a “gene gun” or biolistic particle delivery system. In some embodiments, the rAAV vector is administered via a non-viral lipid nanoparticle. For example, a composition comprising the rAAV vector may comprise one or more diluents, buffers, liposomes, a lipid, a lipid complex. In some embodiments, the rAAV vector is comprised within a microsphere or a nanoparticle, such as a lipid nanoparticle or an inorganic nanoparticle.

In some embodiments, a rAAV is pseudotyped. A pseudotyped rAAV is an infectious virus comprising any combination of an AAV capsid protein and a rAAV genome. Pseudotyped rAAV are useful to alter the tissue or cell specificity of rAAV, and may be employed alone or in conjunction with non-pseudotyped rAAV to transfer one or more genes to a cell, e.g., a mammalian cell. For example, pseudotyped rAAV may be employed subsequent to administration with non-pseudotyped rAAV in a mammal which has developed an immune response to the non-pseudotyped rAAV. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 ITRs, each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype.

In some embodiments, the rAAV vector is an AAV1, AAV2, AAV3, AAV4, AAV5, AAV6, AAV7, AAV8, AAV9, AAV10, or AAV11 vector. In some embodiments, the rAAV vector is AAV1. In some embodiments, the rAAV vector is AAV2. In some embodiments, the rAAV vector is AAV3. In some embodiments, the rAAV vector is AAV4. In some embodiments, the rAAV vector is AAV5. In some embodiments, the rAAV vector is AAV6. In some embodiments, the rAAV vector is AAV7. In some embodiments, the rAAV vector is AAV8. In some embodiments, the rAAV vector is AAV9. In some embodiments, the rAAV vector is AAV10. In some embodiments, the rAAV vector is AAV11. In some embodiments, the rAAV vector is sequence optimized. In some embodiments, the rAAV capsid is modified. For example, in some embodiments, the rAAV8 capsid is modified.

### Age-Related Macular Degeneration (AMD)

In one aspect, the present invention provides, among other things, a method of treating an age-related macular degeneration (AMD) comprising administering an anti-CSF1R sdAb, thereby reducing one or more symptoms related to AMD.

Age-related macular degeneration (AMD) affects one in eight people 60 years of age or older and is the most common cause of irreversible blindness in older persons in developed countries.

The macula is a 5.5-mm-diameter circular patch with a center that is 17 degrees or 4-5 mm temporal and 0.53-0.8 mm inferior to the center of the optic disc. In the human eye, a small center pit called the fovea centralis is made up of several closely spaced cones. For acute center vision, we can thank the fovea. The parafoveal belt and the perifoveal outer zone encircle the fovea. When a person is over 55, macular degeneration is the most common factor contributing to severe, permanent vision loss. The macula deteriorates, which causes it. Age-related macular degeneration (AMD) is a common name for the condition since it occurs as a person ages. The most common reason for elderly people in industrialized nations to lose their vision is age-related macular degeneration (AMD). Geographic atrophy (GA) is one early and non-exudative symptom of AMD that has not yet received disease-specific treatment, despite the fact that anti-vascular endothelial growth factor (VEGF) therapy has significantly improved outcomes.

AMD has been classified into both “dry” and “wet” (exudative, or neovascular) forms. Dry AMD is much more common than wet AMD, but the dry form can progress to the wet form, and the two occur simultaneously in a significant number of cases. Dry AMD is typically characterized by progressive apoptosis of cells in the RPE layer, overlying photoreceptor cells, and frequently also the underlying cells in the choroidal capillary layer. Confluent areas (typically at least 175 m in minimum diameter) of RPE cell death accompanied by overlying photoreceptor atrophy are referred to as geographic atrophy. Patients with this form of AMD experience a slow and progressive deterioration in central vision.

Dry AMD and the deterioration of the RPE layer has been connected to the reduction of certain retinal marker genes (e.g., OPNISW and RPE65). In some embodiments, the anti-CSF1R sdAb described herein protects RPE cells from cell death. In some embodiments, the anti-CSF1R sdAb described herein inhibits the reduction of retinal gene marker expression. In some embodiments, the retinal gene marker is RPE65. In some embodiments, the retinal gene marker is OPNISW.

Wet AMD is characterized by bleeding and/or leakage of fluid from abnormal vessels that have grown from the choroidal vessels (choriocapillaris) beneath the RPE and the macula, which can be responsible for sudden and disabling loss of vision. It has been estimated that much of the vision loss that patients experience is due to such choroidal neovascularization (CNV) and its secondary complications. A subtype of neovascular AMD in which angiomatous proliferation originates from the retina and extends posteriorly into the subretinal space, eventually communicating in some cases with choroidal new vessels has been identified (Yannuzzi, L. A., et al., Retina, 21(5):416-34, 2001). This form of neovascular ARMD, termed retinal angiomatous proliferation (RAP) can be particularly severe. The existence of macular drusen is a strong risk factor for the development of both wet and dry forms of ARMD (Ambati, J., et al., supra).

### Pharmaceutical Compositions and Administration

The sdAbs or the sdAb-Fc fusions of the invention (also referred to herein as “active compounds”), and derivatives, fragments, analogs and homologs thereof, can be incorporated into pharmaceutical compositions suitable for administration. Such compositions typically comprise the sdAbs or the sdAb-Fc fusions and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intravitreal, and rectal administration. Solutions or suspensions used for parenteral, intradermal, or subcutaneous application can include the following components: a sterile diluent such as sterile water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled-release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### EMBODIMENTS

Also disclosed with regard to the above-described subject matter is the following:
A1. A single domain antibody (sdAb) that binds to human CSF1R.
A1-1. A sdAb that binds to cynomolgus CSF1R.
A1-2. A sdAb that binds to human CSF1R and cynomolgus CSF1R.
A1-3. The sdAb of any one of A1 to A1-2, wherein the sdAb does not bind to mouse CSF1R.
A2. A sdAb that binds to D5 of human CSF1R
A2-1. The sdAb of A2, wherein the sdAb binds to contiguous residues of D5.
A2-2. The sdAb of A2, wherein the sdAb binds to non-contiguous residues of D5.
A2-3. The sdAb of any one of A2, A2-1, or A2-2, wherein the sdAb binds to mouse CSF1R of which D5 is replaced with the corresponding domain of human CSF1R.
A2-4. A sdAb that binds to a polypeptide consisting of the amino acid sequence of SEQ ID NO: 161.
A2-5. A sdAb that does not bind to a polypeptide consisting of the amino acid sequence of SEQ ID NO: 160, but does bind to a polypeptide consisting of the amino acid sequence of SEQ ID No: 161.
A2-6. A sdAb that binds to one or more amino acid residues selected from P402, E403, V404, S405, V406, I407, W408, L417, A420, A421, S422, H437, T438, D439, R440, K465, T467, G494, S495, W496, A497, F498, I499, and P500 of human CSF1R (SEQ ID NO: 142), or a combination thereof.
A2-7. A sdAb that binds to one or more amino acid residues selected from I407, A420, W496, and A497 of human CSF1R (SEQ ID NO: 142), or a combination thereof.
A2-8. The sdAb of A2-6, wherein the sdAb binds to two amino acid residues selected from I407, A420, W496, and A497 of human CSF1R (SEQ ID NO: 142).
A2-9. A sdAb that binds to one or more amino acid residues selected from I407, A420, W496, A497, and 1499 of human CSF1R (SEQ ID NO: 142), or a combination thereof.
A2-10. A sdAb that binds to amino acid residues W496, A497, and 1499 of human CSF1R (SEQ ID NO: 142).
A2-11. A sdAb that binds to amino acid residues W496 and A497 of human CSF1R (SEQ ID NO: 142).
A2-12. The sdAb of A2-11, wherein the sdAb does not bind to a CSF1R variant consisting of amino acid substitutions W496N and A497Y relative to SEQ ID NO: 142.
A2-13. A sdAb that binds to amino acid residues A420 and I407 of human CSF1R (SEQ ID NO: 142).
A2-14. The sdAb of A2-13, wherein the sdAb does not bind to a CSF1R variant consisting of amino acid substitutions A420D and I407T relative to SEQ ID NO: 142.
A2-15. The sdAb of any one of A2-7 to A2-14, wherein the sdAb further binds to one or more amino acid residues selected from P402, E403, V404, S405, V406, W408, L417, A421, S422, H437, T438, D439, R440, K465, T467, G494, S495, F498, and P500 of human CSF1R (SEQ ID NO: 142), or a combination thereof.
A2-16. A sdAb that binds to a polypeptide consisting of the amino acid sequence of SEQ ID NO: 161 with greater affinity than to a polypeptide consisting of the amino acid sequence of SEQ ID NO: 160.
A2-17. A sdAb that binds to a polypeptide consisting of the amino acid sequence of SEQ ID NO: 161 with more than 10-fold greater affinity than to a polypeptide consisting of the amino acid sequence of SEQ ID NO: 160.
B1. A sdAb that binds to human CSF1R, wherein the sdAb comprises
   a. a CDR1 comprising an amino acid sequence that is identical to or differs by no more than 2 amino acid residues from GGTFSYY (SEQ ID NO: 106), a CDR2 comprising an amino acid sequence that is identical to or differs by no more than 2 amino acid residues from SQYGSN (SEQ ID NO: 109), and a CDR3 comprising an amino acid sequence that is identical to or differs by no more than 2 amino acid residues from LRSGSDWRTIRDYDN (SEQ ID NO: 112); or
   b. a CDR1 comprising an amino acid sequence that is identical to or differs by no more than 2 amino acid residues from AASGGTFSYYNMG (SEQ ID NO: 124), a CDR2 comprising an amino acid sequence that is identical to or differs by no more than 2 amino acid residues from VISQYGSNTY (SEQ ID NO: 127), and a CDR3 comprising an amino acid sequence that is identical to or differs by no more than 2 amino acid residues from ATLRSGSDWRTIRDYDN (SEQ ID NO: 130); or
   c. a CDR1 comprising an amino acid sequence that is identical to or differs by no more than 2 amino acid residues from GGTFSYYN (SEQ ID NO: 115), a CDR2 comprising an amino acid sequence that is identical to or differs by no more than 2 amino acid residues from ISQYGSNT (SEQ ID NO: 118), and a CDR3 comprising an amino acid sequence that is identical to or differs by no more than 2 amino acid residues from ATLRSGSDWRTIRDYDN (SEQ ID NO: 121); or
   d. a CDR1 comprising an amino acid sequence that is identical to or differs by no more than 2 amino acid residues from YYNMG (SEQ ID NO: 133), a CDR2 comprising an amino acid sequence that is identical to or differs by no more than 2 amino acid residues from VISQYGSNTYYADSVKG (SEQ ID NO: 136), and a CDR3 comprising an amino acid sequence that is identical to or differs by no more than 2 amino acid residues from LRSGSDWRTIRDYDN (SEQ ID NO: 139); or
   e. a CDR1 comprising an amino acid sequence that is identical to or differs by no more than 2 amino acid residues from GDIFSFN (SEQ ID NO: 107), a CDR2 comprising an amino acid sequence that is identical to or differs by no more than 2 amino acid residues from APGGN (SEQ ID NO: 110), and a CDR3 comprising an amino acid sequence that is identical to or differs by no more than 2 amino acid residues from WPFSRDS (SEQ ID NO: 113); or
   f. a CDR1 comprising an amino acid sequence that is identical to or differs by no more than 2 amino acid residues from AASGDIFSFNVMG (SEQ ID NO: 125), a CDR2 comprising an amino acid sequence that is identical to or differs by no more than 2 amino acid residues from TIAPGGNTN (SEQ ID NO: 156), and a CDR3 comprising an amino acid sequence that is identical to or differs by no more than 2 amino acid residues from NRWPFSRDS (SEQ ID NO: 131); or
   g. a CDR1 comprising an amino acid sequence that is identical to or differs by no more than 2 amino acid residues from AASGDIFSFNVMG (SEQ ID NO: 125), a CDR2 comprising an amino acid sequence that is identical to or differs by no more than 2 amino acid residues from VIAPGGNTY (SEQ ID NO: 128), and a CDR3 comprising an amino acid sequence that is identical to or differs by no more than 2 amino acid residues from NRWPFSRDS (SEQ ID NO: 131); or
   h. a CDR1 comprising an amino acid sequence that is identical to or differs by no more than 2 amino acid residues from GDIFSFNV (SEQ ID NO: 116), a CDR2 comprising an amino acid sequence that is identical to or differs by no more than 2 amino acid residues from IAPGGNT (SEQ ID NO: 119), and a CDR3 comprising an amino acid sequence that is identical to or differs by no more than 2 amino acid residues from NRWPFSRDS (SEQ ID NO: 122); or
   i. a CDR1 comprising an amino acid sequence that is identical to or differs by no more than 2 amino acid residues from FNVMG (SEQ ID NO: 134), a CDR2 comprising an amino acid sequence that is identical to or differs by no more than 2 amino acid residues from TIAPGGNTNYFDSARG (SEQ ID NO: 157), and a CDR3 comprising an amino acid sequence that is identical to or differs by no more than 2 amino acid residues from WPFSRDS (SEQ ID NO: 140); or
   j. a CDR1 comprising an amino acid sequence that is identical to or differs by no more than 2 amino acid residues from FNVMG (SEQ ID NO: 134), a CDR2 comprising an amino acid sequence that is identical to or differs by no more than 2 amino acid residues from VIAPGGNTYYADSVKG (SEQ ID NO: 137), and a CDR3 comprising an amino acid sequence that is identical to or differs by no more than 2 amino acid residues from WPFSRDS (SEQ ID NO: 140); or
   k. a CDR1 comprising an amino acid sequence that is identical to or differs by no more than 2 amino acid residues from GMAFGSS (SEQ ID NO: 108), a CDR2 comprising an amino acid sequence that is identical to or differs by no more than 2 amino acid residues from TSAGY (SEQ ID NO: 111), and a CDR3 comprising an amino acid sequence that is identical to or differs by no more than 2 amino acid residues from GPFHSTAYST (SEQ ID NO: 114); or
   l. a CDR1 comprising an amino acid sequence that is identical to or differs by no more than 2 amino acid residues from VASGMAFGSSPMN (SEQ ID NO: 126), a CDR2 comprising an amino acid sequence that is identical to or differs by no more than 2 amino acid residues from GVTSAGYAY (SEQ ID NO: 129), and a CDR3 comprising an amino acid sequence that is identical to or differs by no more than 2 amino acid residues from ARGPFHSTAYST (SEQ ID NO: 132); or
   m. a CDR1 comprising an amino acid sequence that is identical to or differs by no more than 2 amino acid residues from GMAFGSSP (SEQ ID NO: 117), a CDR2 comprising an amino acid sequence that is identical to or differs by no more than 2 amino acid residues from VTSAGYA (SEQ ID NO: 120), and a CDR3 comprising an amino acid sequence that is identical to or differs by no more than 2 amino acid residues from ARGPFHSTAYST (SEQ ID NO: 123); or
   n. a CDR1 comprising an amino acid sequence that is identical to or differs by no more than 2 amino acid residues from SSPMN (SEQ ID NO: 135), a CDR2 comprising an amino acid sequence that is identical to or differs by no more than 2 amino acid residues from GVTSAGYAYYADWVKG (SEQ ID NO: 138), and a CDR3 comprising an amino acid sequence that is identical to or differs by no more than 2 amino acid residues from GPFHSTAYST (SEQ ID NO: 141).
B2. A sdAb that binds to human CSF1R, wherein the sdAb comprises,
   a. a CDR1 comprising an amino acid sequence of GGTFSYY (SEQ ID NO: 106), a CDR2 comprising an amino acid sequence of SQYGSN (SEQ ID NO: 109), and a CDR3 comprising an amino acid sequence of LRSGSDWRTIRDYDN (SEQ ID NO: 112); or
   b. a CDR1 comprising an amino acid sequence of AASGGTFSYYNMG (SEQ ID NO: 124), a CDR2 comprising an amino acid sequence of VISQYGSNTY (SEQ ID NO: 127), and a CDR3 comprising an amino acid sequence of ATLRSGSDWRTIRDYDN (SEQ ID NO: 130); or
   c. a CDR1 comprising an amino acid sequence of GGTFSYYN (SEQ ID NO: 115), a CDR2 comprising an amino acid sequence of ISQYGSNT (SEQ ID NO: 118), and a CDR3 comprising an amino acid sequence of ATLRSGSDWRTIRDYDN (SEQ ID NO: 121); or
   d. a CDR1 comprising an amino acid sequence of YYNMG (SEQ ID NO: 133), a CDR2 comprising an amino acid sequence of VISQYGSNTYYADSVKG (SEQ ID NO: 136), and a CDR3 comprising an amino acid sequence of LRSGSDWRTIRDYDN (SEQ ID NO: 139); or
   e. a CDR1 comprising an amino acid sequence of GDIFSFN (SEQ ID NO: 107), a CDR2 comprising an amino acid sequence of APGGN (SEQ ID NO: 110), and a CDR3 comprising an amino acid sequence of WPFSRDS (SEQ ID NO: 113); or
   f. a CDR1 comprising an amino acid sequence of AASGDIFSFNVMG (SEQ ID NO: 125), a CDR2 comprising an amino acid sequence of TIAPGGNTN (SEQ ID NO: 156), and a CDR3 comprising an amino acid sequence of NRWPFSRDS (SEQ ID NO: 131); or
   g. a CDR1 comprising an amino acid sequence of AASGDIFSFNVMG (SEQ ID NO: 125), a CDR2 comprising an amino acid sequence of VIAPGGNTY (SEQ ID NO: 128), and a CDR3 comprising an amino acid sequence of NRWPFSRDS (SEQ ID NO: 131); or
   h. a CDR1 comprising an amino acid sequence of GDIFSFNV (SEQ ID NO: 116), a CDR2 comprising an amino acid sequence of IAPGGNT (SEQ ID NO: 119), and a CDR3 comprising an amino acid sequence of NRWPFSRDS (SEQ ID NO: 122); or
   i. a CDR1 comprising an amino acid sequence of FNVMG (SEQ ID NO: 134), a CDR2 comprising an amino acid sequence of TIAPGGNTNYFDSARG (SEQ ID NO: 157), and a CDR3 comprising an amino acid sequence of WPFSRDS (SEQ ID NO: 140); or
   j. a CDR1 comprising an amino acid sequence of FNVMG (SEQ ID NO: 134), a CDR2 comprising an amino acid sequence of VIAPGGNTYYADSVKG (SEQ ID NO: 137), and a CDR3 comprising an amino acid sequence of WPFSRDS (SEQ ID NO: 140); or
   k. a CDR1 comprising an amino acid sequence of GMAFGSS (SEQ ID NO: 108), a CDR2 comprising an amino acid sequence of TSAGY (SEQ ID NO: 111), and a CDR3 comprising an amino acid sequence of GPFHSTAYST (SEQ ID NO: 114); or
   l. a CDR1 comprising an amino acid sequence of VASGMAFGSSPMN (SEQ ID NO: 126), a CDR2 comprising an amino acid sequence of GVTSAGYAY (SEQ ID NO: 129), and a CDR3 comprising an amino acid sequence of ARGPFHSTAYST (SEQ ID NO: 132); or
   m. a CDR1 comprising an amino acid sequence of GMAFGSSP (SEQ ID NO: 117), a CDR2 comprising an amino acid sequence of VTSAGYA (SEQ ID NO: 120), and a CDR3 comprising an amino acid sequence of ARGPFHSTAYST (SEQ ID NO: 123); or
   n. a CDR1 comprising an amino acid sequence of SSPMN (SEQ ID NO: 135), a CDR2 comprising an amino acid sequence of GVTSAGYAYYADWVKG (SEQ ID NO: 138), and a CDR3 comprising an amino acid sequence of GPFHSTAYST (SEQ ID NO: 141).
B3. A sdAb that binds to CSF1R, wherein the sdAb comprises,
   a. a CDR1 consisting of an amino acid sequence of GGTFSYY (SEQ ID NO: 106), a CDR2 consisting of an amino acid sequence of SQYGSN (SEQ ID NO: 109), and a CDR3 consisting of an amino acid sequence of LRSGSDWRTIRDYDN (SEQ ID NO: 112); or
   b. a CDR1 consisting of an amino acid sequence of AASGGTFSYYNMG (SEQ ID NO: 124), a CDR2 consisting of an amino acid sequence of VISQYGSNTY (SEQ ID NO: 127), and a CDR3 consisting of an amino acid sequence of ATLRSGSDWRTIRDYDN (SEQ ID NO: 130); or
   c. a CDR1 consisting of an amino acid sequence of GGTFSYYN (SEQ ID NO: 115), a CDR2 consisting of an amino acid sequence of ISQYGSNT (SEQ ID NO: 118), and a CDR3 consisting of an amino acid sequence of ATLRSGSDWRTIRDYDN (SEQ ID NO: 121); or
   d. a CDR1 consisting of an amino acid sequence of YYNMG (SEQ ID NO: 133), a CDR2 consisting of an amino acid sequence of VISQYGSNTYYADSVKG (SEQ ID NO: 136), and a CDR3 consisting of an amino acid sequence of LRSGSDWRTIRDYDN (SEQ ID NO: 139); or
   e. a CDR1 consisting of an amino acid sequence of GDIFSFN (SEQ ID NO: 107), a CDR2 consisting of an amino acid sequence of APGGN (SEQ ID NO: 110), and a CDR3 consisting of an amino acid sequence of WPFSRDS (SEQ ID NO: 113); or
   f. a CDR1 consisting of an amino acid sequence of AASGDIFSFNVMG (SEQ ID NO: 125), a CDR2 consisting of an amino acid sequence of TIAPGGNTN (SEQ ID NO: 156), and a CDR3 consisting of an amino acid sequence of NRWPFSRDS (SEQ ID NO: 131); or
   g. a CDR1 consisting of an amino acid sequence of AASGDIFSFNVMG (SEQ ID NO: 125), a CDR2 consisting of an amino acid sequence of VIAPGGNTY (SEQ ID NO: 128), and a CDR3 consisting of an amino acid sequence of NRWPFSRDS (SEQ ID NO: 131); or
   h. a CDR1 consisting of an amino acid sequence of GDIFSFNV (SEQ ID NO: 116), a CDR2 consisting of an amino acid sequence of IAPGGNT (SEQ ID NO: 119), and a CDR3 consisting of an amino acid sequence of NRWPFSRDS (SEQ ID NO: 122); or
   i. a CDR1 consisting of an amino acid sequence of FNVMG (SEQ ID NO: 134), a CDR2 consisting of an amino acid sequence of TIAPGGNTNYFDSARG (SEQ ID NO: 157), and a CDR3 consisting of an amino acid sequence of WPFSRDS (SEQ ID NO: 140); or
   j. a CDR1 consisting of an amino acid sequence of FNVMG (SEQ ID NO: 134), a CDR2 consisting of an amino acid sequence of VIAPGGNTYYADSVKG (SEQ ID NO: 137), and a CDR3 consisting of an amino acid sequence of WPFSRDS (SEQ ID NO: 140); or
   k. a CDR1 consisting of an amino acid sequence of GMAFGSS (SEQ ID NO: 108), a CDR2 consisting of an amino acid sequence of TSAGY (SEQ ID NO: 111), and a CDR3 consisting of an amino acid sequence of GPFHSTAYST (SEQ ID NO: 114); or
   l. a CDR1 consisting of an amino acid sequence of VASGMAFGSSPMN (SEQ ID NO: 126), a CDR2 consisting of an amino acid sequence of GVTSAGYAY (SEQ ID NO: 129), and a CDR3 consisting of an amino acid sequence of ARGPFHSTAYST (SEQ ID NO: 132); or
   m. a CDR1 consisting of an amino acid sequence of GMAFGSSP (SEQ ID NO: 117), a CDR2 consisting of an amino acid sequence of VTSAGYA (SEQ ID NO: 120), and a CDR3 consisting of an amino acid sequence of ARGPFHSTAYST (SEQ ID NO: 123); or
   n. a CDR1 consisting of an amino acid sequence of SSPMN (SEQ ID NO: 135), a CDR2 consisting of an amino acid sequence of GVTSAGYAYYADWVKG (SEQ ID NO: 138), and a CDR3 consisting of an amino acid sequence of GPFHSTAYST (SEQ ID NO: 141).
B4. A sdAb that binds to CSF1R, wherein the sdAb comprises an amino acid sequence at least 85%, 90%, 95%, 96%, 97%, 98%, or 99% identical to any one of SEQ ID NOs: 1-3 or 101-105.
B5. A sdAb that binds to CSF1R, wherein the sdAb comprises an amino acid sequence identical to any one of SEQ ID NOs: 1-3 or 101-105.
B6. A sdAb that binds to CSF1R, wherein the sdAb having an amino acid sequence identical to any one of SEQ ID NOs: 1-3 or 101-105.
C1. A sdAb that binds to CSF1R, wherein the sdAb comprises
   a. a CDR1 comprising a consensus sequence of GX₁TFSX₂YN (SEQ ID NO: 147), wherein X₁ comprises G, L, or F, and X₂ comprises S or Y;
   b. a CDR2 comprising an amino acid sequence of ISQYGSNT (SEQ ID NO: 150); and
   c. a CDR3 comprising a consensus sequence of TLRSGSDWRTX₃RDYDNW (SEQ ID NO: 153), wherein X₃ comprises I or M.
C2. A sdAb that binds to CSF1R, wherein the sdAb comprises
   a. a CDR1 comprising a consensus sequence of GX₄X₅FGSX₆ (SEQ ID NO: 148), wherein X₄ comprises F, M, or L, X₅ comprises T or A, and X₆ comprises T or S;
   b. a CDR2 comprising a consensus sequence of X₇TSX₈GYX₉ (SEQ ID NO: 151), wherein X₇ comprises V or T, X₈ comprises S, A, or G, and X₉ comprises S, A, or T; and
   c. a CDR3 comprising a consensus sequence of RGPFHSX₁₀AYX₁₁TR (SEQ ID NO: 154), wherein X₁₀ comprises T or I, and X₁₁ comprises A or S.
C3. A sdAb that binds to CSF1R, wherein the sdAb comprises
   a. a CDR1 comprising a consensus sequence of GX₂₁IFX₂₂X₂₃X₂₄V (SEQ ID NO: 149), wherein X₂₁ comprises D or N, X₂₂ comprises R or S, X₂₃ comprises F or S, and X₂₄ comprises T or N;
   b. a CDR2 comprising a consensus sequence of IX₂₅X₂₆GGX₂₇T (SEQ ID NO: 152), wherein X₂₅ comprises T, A, or S, X₂₆ comprises S, P, or T, and X₂₇ comprises R, N, or K; and
   c. a CDR3 comprising a consensus sequence of X₂₈LPFX₂₉RDS (SEQ ID NO: 155), wherein X₂₈ comprises R, T, or S, and X₂₉ comprises R or S.
D1. The sdAb of any one of A1 to C3, wherein the sdAb is a llama VHH or a humanized VHH.
E1. The sdAb of any one of A1 to D1, wherein the sdAb is fused to an Fc region.
E1-1. The sdAb of E1, wherein the Fc region is derived from IgG1, IgG2, IgG3, or IgG4.
E1-2. The sdAb of E1 or E1-1, wherein the Fc region is modified.
F1. The sdAb of any one of A1 to E1-2, wherein the sdAb binds to human CSF1R with a binding affinity (KD) between 1 and 12 nM.
F1-1. The sdAb of any one of A1 to E1-2, wherein the sdAb binds to cynomolgus CSF1R with a binding affinity (K_{D}) of between 2 and 6 nM.
F1-2. The sdAb of any one of A1 to F1-1, wherein the sdAb is anti-CSF1R sdAb. F2. The sdAb of any one of A1 to F1-2, wherein the sdAb inhibits CSF1 activity with an IC₅₀ value between 0.3 and 4 nM.
F2-1. The sdAb of any one of A1 to F2, wherein the sdAb inhibits IL-34 activity with an IC₅₀ value between 1.5 and 22 nM.
F2-2. The sdAb of D2 or F2-1, wherein the IC₅₀ value is determined by cellular reporter assay or macrophage proliferation assay.
G1. A nucleic acid encoding the sdAb of any one of A1 to F2-2.
H1. A vector comprising the nucleic acid of G1.
H2. The vector of H1, wherein the vector is a viral vector.
H3. The vector of H2, wherein the viral vector is an adeno-associate virus (AAV) vector.
H4. The vector of H3, wherein the AAV vector is selected from AAV1, AAV2, AAV3, AAV4, AAV5, AAV6, AAV7, AAV8, AAV9, AAV10, AAV10, AAV11, AAV12, AAV13.
I1. A liquid nanoparticle (LNP) comprising the nucleic acid of G1 or the vector of any one of H1 to H4.
J1. A host cell comprising the nucleic acid of G1 or the vector of any one of H1 to H4.
J2. A method of producing a sdAb, comprising culturing the host cell of J1 under conditions that allow production of the sdAb.
J3. The method of J2, further comprising isolating sdAb.
K1. A pharmaceutical composition comprising
   a. the sdAb of any one of A1 to F2-2, the nucleic acid of G1, the vector of any one of H1 to H4, the LNP of I1, or the host cell of J1, and
   b. one or more physiologically acceptable carriers, excipients, or diluents.
L1. The sdAb of any one of A1 to F2-2, the nucleic acid of G1, the vector of any one of H1 to H4, the LNP of I1, or the host cell of J1, for use in inhibiting CSF1R signal.
L1-1. The sdAb, the nucleic acid, the vector, the LNP or the host cell of L1, further for use in inhibiting macrophage proliferation.
L1-2. The sdAb, the nucleic acid, the vector, the LNP or the host cell of L1 or L1-1, further for use in treating a disease or disorder associated with CSF1R.
L2. The sdAb of any one of A1 to F2-2, the nucleic acid of G1, the vector of any one of H1 to H4, the LNP of I1, or the host cell of J1, for use in inhibiting macrophage proliferation.
L2-1. The sdAb, the nucleic acid, the vector, the LNP or the host cell of L2, further for use in treating a disease or disorder associated with CSF1R.
L3. The sdAb of any one of A1 to F2-2, the nucleic acid of G1, the vector of any one of H1 to H4, the LNP of I1, or the host cell of J1, for use in treating a disease or disorder associated with CSF1R.
L4. The sdAb, the nucleic acid, the vector, the LNP or the host cell of any one of L1-2, L2-1 and L3, wherein the disease or disorder associated with CSF1R is an ocular disease or disorder.
L5. The sdAb, the nucleic acid, the vector, the LNP or the host cell of L4, wherein the disease or disorder associated with CSF1R is AMD.
L5-1. The sdAb, the nucleic acid, the vector, the LNP or the host cell of L5, wherein AMD is dry AMD.
M1. Use of any one of A1 to F2-2, the nucleic acid of G1, the vector of any one of H1 to H4, the LNP of I1, or the host cell of J1, in the manufacture of a medicament for treatment of a disease or disorder associated with CSF1R.
M1-1. The use of M1, wherein the disease or disorder associated with CSF1R is Age-related macular degeneration (AMD), Stargardt disease, Retinitis pigmentosa, Central retinal vein occlusion (CRVO), Choroidal neovascularization (CNV), Diabetic retinopathy, Proliferative diabetic retinopathy (PDR), Proliferative vitreoretinopathy, Retinal edema, Retinal vein occlusion (RVO), Retinopathy of prematurity (ROP), Sequela associated with retinal ischemia,
Anterior segment neovascularization, Corneal neovascularization, Posterior segment neovascularization, Polypoidal choroidal vasculopathy, Diabetic macular edema (DME), Noninfectious uveitis (NIU), Uveitis, Dry eye syndrome, or Glaucoma.
N1. The pharmaceutical composition of K1, for use in treatment of a disease or disorder associated with CSF1R.
O1. A method of inhibiting CSF1R signal in a subject in need thereof, comprising administering to the subject the sdAb of any one of A1 to F2-2 or the pharmaceutical composition of K1.
O2. A method of inhibiting macrophage proliferation in a subject in need thereof, comprising administering to the subject the sdAb of any one of A1 to F2-2 or the pharmaceutical composition of K1.
O3. A method of treating a disease or disorder associated with CSF1R in a subject in need thereof, comprising administering to the subject the sdAb of any one of A1 to F2-2 or the pharmaceutical composition of K1.
O4-1. A method of inhibiting CSF1R signal in a subject in need thereof, comprising delivering to the subject the sdAb of any one of A1 to F2-2.
O4-2. A method of inhibiting macrophage proliferation in a subject in need thereof, comprising delivering to the subject the sdAb of any one of A1 to F2-2.
O4-3. A method of treating a disease or disorder associated with CSF1R in a subject in need thereof, comprising delivering to the subject the sdAb of any one of A1 to F2-2.
O4-4. The method of any one of 04-1 to 04-3, wherein the method further comprises administering the nucleic acid of G1, the vector of any one of H1 to H4, the LNP of I1, or the host cell of J1.
O5. The method of any one of 01, 02, 04-1 and 04-2, wherein the method further comprises treating a disease or disorder associated with CSF1R in a subject in need thereof.
O6. The method of any one of 03, 04-3 and 04-4, wherein the disease or disorder associated with CSF1R is an ocular disease or disorder.
O6-1. The method of any one of 03, 04-3, and 04-4, wherein the disease or disorder associated with CSF1R is Age-related macular degeneration (AMD), Stargardt disease, Retinitis pigmentosa, Central retinal vein occlusion (CRVO), Choroidal neovascularization (CNV), Diabetic retinopathy, Proliferative diabetic retinopathy (PDR), Proliferative vitreoretinopathy, Retinal edema, Retinal vein occlusion (RVO), Retinopathy of prematurity (ROP), Sequela associated with retinal ischemia, Anterior segment neovascularization, Corneal neovascularization, Posterior segment neovascularization, Polypoidal choroidal vasculopathy, Diabetic macular edema (DME), Noninfectious uveitis (NIU), Uveitis, Dry eye syndrome, or Glaucoma.
O7. The method of 06 or 06-1, wherein the disease or disorder associated with CSF1R is AMD.
O7-1. The method of 07, wherein AMD is dry AMD.
P1. A CSF1R inhibitor for use in treating dry AMD.
P2. The CSF1R inhibitor of P1, wherein the CSF1R inhibitor is an anti-CSF1R antibody.
Q1. A polypeptide that comprises a first sdAb and a second sdAb, wherein the first sdAb and the second sdAb are any one of the sdAb of A1 to F2-2.
Q2. The polypeptide of Q1, wherein the first sdAb and the second sdAb are fused to a half-life extension domain.
Q3. The polypeptide of Q1 or Q2, wherein the first sdAb and the second sdAb are fused to an Fc region.
Q4. The polypeptide of Q3, wherein the Fc region is bivalent.
Q5. The polypeptide of Q1 or Q2, wherein the first sdAb and the second sdAb are fused to a peptide that binds to human serum albumin.
Q6. The polypeptide of Q5, wherein the peptide that binds to human serum albumin is an sdAb.
Q7. The polypeptide of any one of Q1 to Q6, wherein the first sdAb comprises the same amino acid sequence as the second sdAb.
Q8. The polypeptide of any one of Q1 to Q6, wherein the first sdAb comprises a different amino acid sequence from the second sdAb.

### EXAMPLES

Other features, objects, and advantages of the present disclosure are apparent in the examples that follow. It should be understood, however, that the examples, while indicating embodiments of the present disclosure, are given by way of illustration only, not limitation. Various changes and modifications within the scope of the disclosure will become apparent to those skilled in the art from the examples.

### Example 1. Anti-CSF1R sdAb/VHH Binding Assays

This example illustrates that exemplary anti-CSF1R sdAbs of the present invention bind to both human CSF1R and cynomolgus monkey CSF1R with high affinity.

### Methods for Binding Assays

A CM5 chip (Cytiva) was installed in the Biacore 8k (Cytiva). Using either the His-tag Capture Kit or the Human Antibody Capture Kit (both from Cytiva), anti-His-tagged sdAbs or monovalent Fc-fused sdAbs were coupled to the chip following the respective kit protocols. sdAbs and antigens were diluted in 1×HBS-EP+ buffer, which was also used as the running buffer. After multiple rounds of optimization, the final parallel kinetics experiment was conducted by capturing 0.25 μg/mL of His-tagged sdAbs or 0.025 μg/mL of monovalent Fc-fused sdAbs onto the chip for 60 seconds and flowing an 8-point antigen concentration curve over the chip, with each of the 8 flow cell channels having a different concentration. Association was performed for 240 seconds and dissociation was performed for 900 seconds for His-tagged sdAbs and 3600 seconds for monovalent Fc-fused sdAbs. The data was analyzed using Biacore Insight software.

### Results of Binding Assays

As stated above, the binding of the exemplary anti-CSF1R sdAbs was tested via surface plasmon resonance using Biacore in the His-tagged sdAb only and sdAb monovalent-Fc fused antibody formats.

The binding constants (K_{D}) obtained for the His-tagged sdAb domain only format yielded values in the low nanomolar range for all clones against both the human and cynomolgus monkey CSF1R receptors (Table 1).

TABLE 1
Binding Affinities of VHH or sdAbs for CSF1R

K_{D} (nM)

| Clone | Human CSF1R | Cynomolgus Monkey CSF1R |
|---|---|---|
| Ab1 Parental | 6.3 | 2.3 |
| Ab1-9 | 11.4 | 5.9 |
| Ab1-12 | 6.0 | 2.9 |
| Ab1-18 | 5.0 | 2.3 |
| Ab1-20 | 1.8 | 2.4 |
| Ab2-10 | 6.1 | 3.0 |

Additionally, the K_{D} values of the VHH-monoFc (monovalent VHH-Fc fusion) to human and cynomolgus monkey CSF1R were in picomolar ranges (Table 2).

TABLE 2
Binding Affinities of Monovalent VHH-Fc fusion

K_{D} (pM)

| Clone | Human CSF1R | Cynomolgus Monkey CSF1R |
|---|---|---|
| Ab1 Parental | 155 | 209 |
| Ab2 Parental | 88.1 | 70.1 |
| Ab3 Parental | 158 | 515 |

### Example 2. Evaluation of Inhibitory Activity by Anti-CSF1R sdAb Formats

This example illustrates that exemplary anti-CSF1R sdAbs of the present invention inhibit binding of both CSF1 and IL34 to CSF1R with low IC50 values.

### Methods for CSF1R/IL-34 Reporter Inhibitory Assays

CSF1R/SRE reporter cells (BPS Bioscience) were seeded in a 96-well plate at a density of 30,000 cells per well in 100 μL of Thaw Medium 1 (BPS Bioscience) one day before the experiment. The cells were then cultured overnight at 37° C. in a 5% CO₂ incubator. The next day, the Thaw Medium 1 was removed from the wells and replaced with 50 μL of Assay Medium 1B (BPS Bioscience). The cells were incubated for an additional 20-24 hours under the same conditions. Following the incubation, 50 μL of serially diluted sdAbs and control antibodies were added to the wells. One hour later, CSF1 or IL34 was added to the wells to achieve a final concentration of 30 ng/mL and 75 ng/mL, respectively. The plates were incubated for 6 hours at 37° C. in a 5% CO₂ incubator. After the 6-hour incubation, 100 μL of One-STEP Luciferase reagent (BPS Bioscience) was added to each well. The plates were incubated at room temperature for approximately 15 minutes and luminescence signal was then measured using a SpectraMAX M3 Plate Reader. The resulting data were analyzed using Igor Pro software.

### Results for CSF1R/IL-34 Reporter Inhibitory Assays

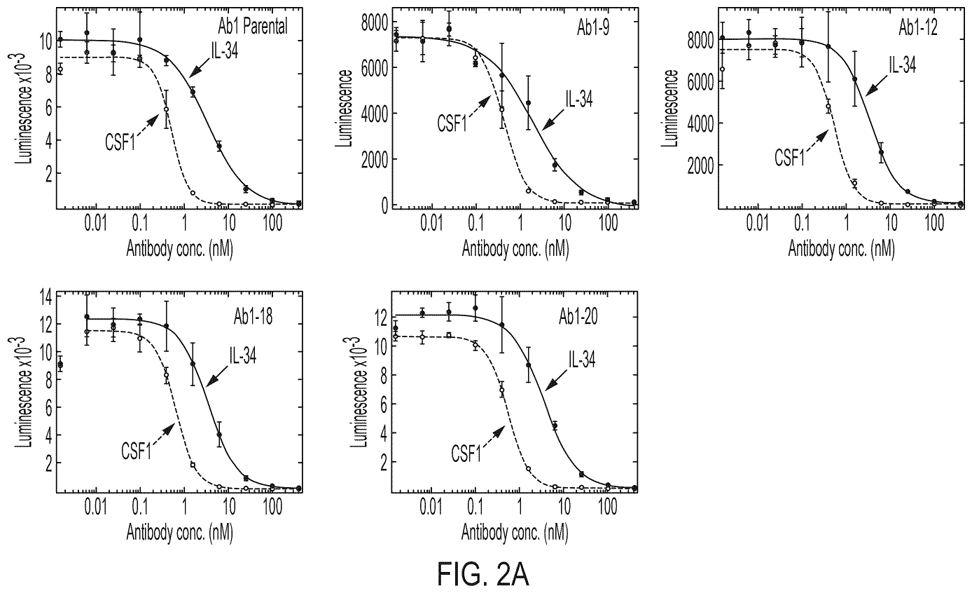

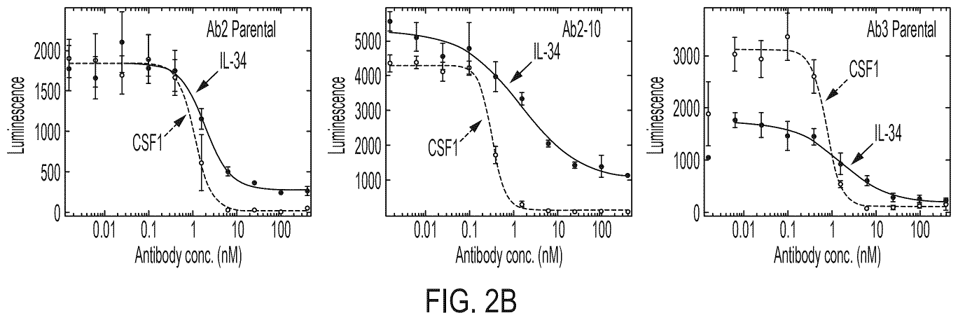

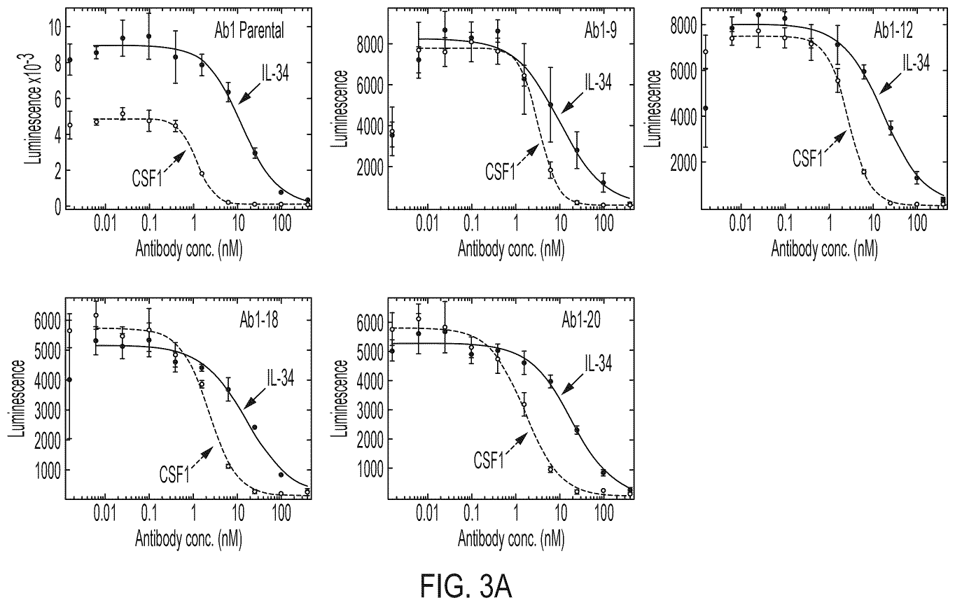

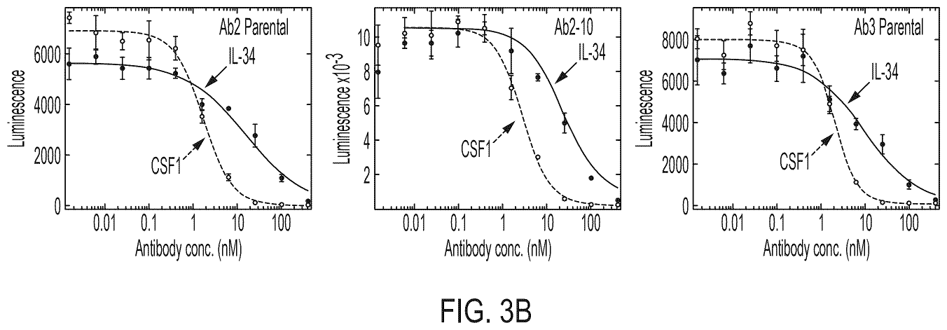

Cellular reporter assays were performed to evaluate the inhibitory activity of the anti-CSF1R sdAbs in the presence of either 30 ng/mL CSF1 or 75 ng/mL IL-34 whilst titrating in various concentrations of said antibodies (FIGS. 2A-2B and FIGS. 3A-3B, respectively for sdAb/VHH alone or sdAb/VHH-monoFc). The IC₅₀ values reported herein were then calculated from these titration curves.

As shown in Table 3, the IC₅₀ values determined for the inhibitory activity of the sdAb format clones against CSF1 ligand range from 0.3 to 1.1 nM, while the range of inhibitory activity against IL-34 ranged from 1.5 to 3.6 nM.

TABLE 3
His-tagged sdAb Format Inhibition Constants
by Cellular Reporter Assay

IC₅₀ (nM)

| Clone | CSF1 | IL34 |
|---|---|---|
| Ab1 Parental | 0.51 | 3.3 |
| Ab1-9 | 0.43 | 2.0 |
| Ab1-12 | 0.54 | 3.5 |
| Ab1-18 | 0.64 | 3.4 |
| Ab1-20 | 0.55 | 3.6 |
| Ab2 Parental | 1.1 | 1.9 |
| Ab2-10 | 0.33 | 1.5 |
| Ab3 Parental | 0.76 | 1.8 |

As shown in Table 4, the IC₅₀ values determined for the inhibitory activity of the VHH/sdAb monovalent Fc-fusion format clones were also in the low nanomolar against both CSF1 and IL-34.

TABLE 4
sdAb Monovalent Fc-Fusion Format Inhibition
Constants by Cellular Reporter Assay

IC₅₀ (nM)

| Clone | CSF1 | IL-34 |
|---|---|---|
| Ab1 Parental | 1.2 | 12.7 |
| Ab1-9 | 3.4 | 10.1 |
| Ab1-12 | 2.8 | 17.4 |
| Ab1-18 | 2.3 | 16.4 |
| Ab1-20 | 1.6 | 18 |
| Ab2 Parental | 1.7 | 15.1 |
| Ab2-10 | 2.8 | 21.8 |
| Ab3 Parental | 2.0 | 9.7 |

### Methods for Macrophage Proliferation Assays

CD14+ monocytes for the macrophage proliferation assay were isolated from peripheral blood collected from healthy volunteers, purchased from Research Blood Components, LLC, using an exemplary protocol. Briefly, peripheral blood mononuclear cells (PBMCs) were isolated by density gradient centrifugation using Lymphoprep (STEMCELL). CD14+ monocytes were then purified from PBMCs using the EasySep Direct Human Monocyte Isolation kit (STEMCELL), following the manufacturer's instructions. The isolated CD14+ monocytes were seeded in a 96-well plate at a density of 50,000 or 100,000 cells per well in 50 μL RPMI-1640 medium (Gibco) supplemented with 10% FBS (Avantor), 100 U/mL penicillin, 100 μg/mL streptomycin (Gibco), and 10 mM HEPES (Gibco). Subsequently, 40 μL of serially diluted His-tagged sdAbs and control antibodies were added to the wells. After a 1-hour incubation at 37° C. in a 5% CO₂ incubator, CSF1 or IL-34 was added to the wells to achieve a final concentration of 50 ng/mL. The plates were then incubated for 6 days at 37° C. in a 5% CO₂ incubator. After the 6-day incubation, 100 μL of CellTiter-Glo 2.0 Assay reagent (Promega) was added to each well. The plates were incubated at room temperature for approximately 15 minutes, and the luminescence signal was measured using a SpectraMAX M3 Plate Reader. The resulting data were analyzed using Igor Pro software.

### Results for Macrophage Proliferation Assays

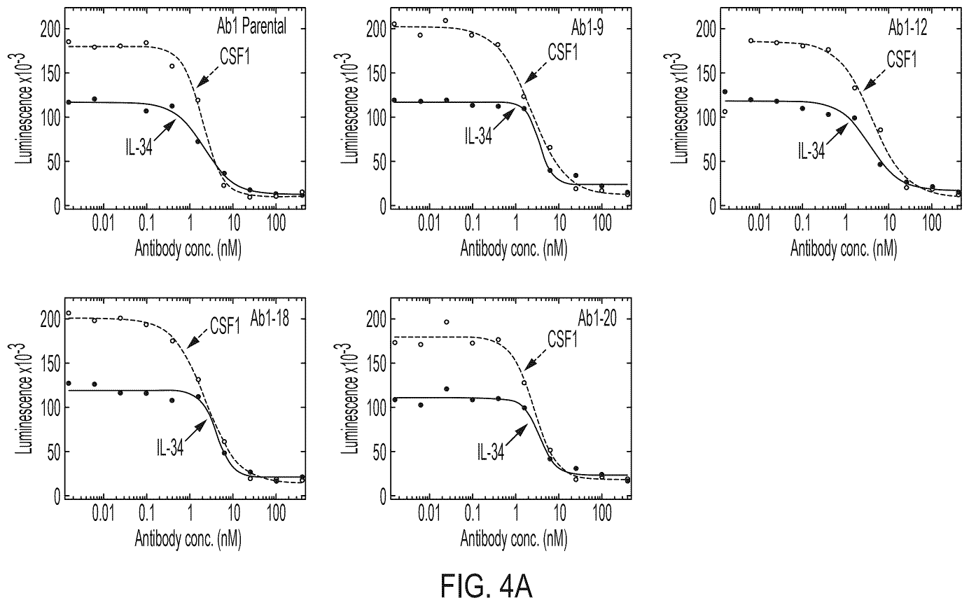

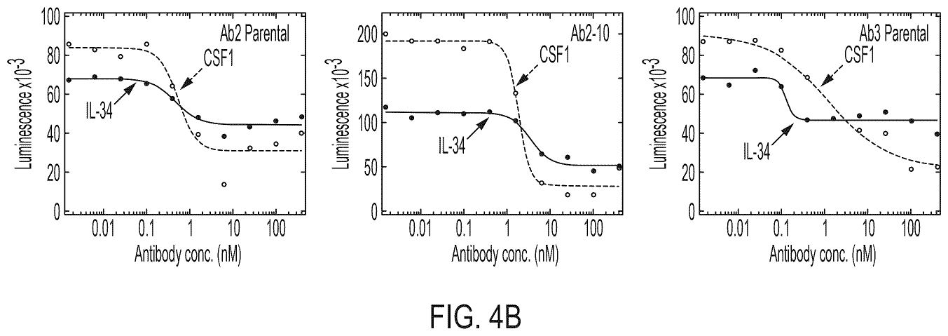

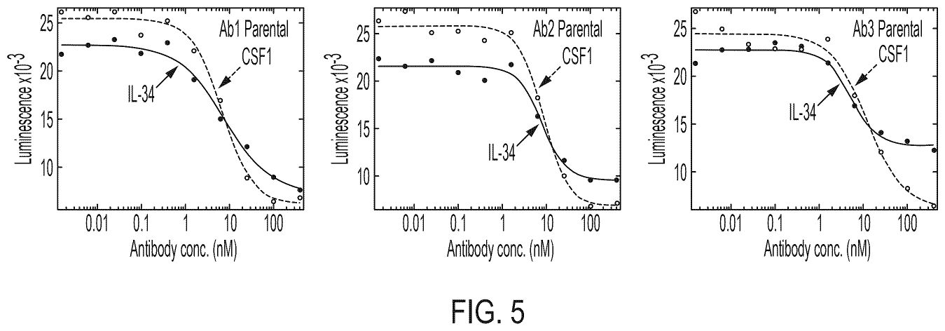

Macrophage proliferation assays were performed to evaluate the inhibitory activity of the anti-CSF1R sdAbs in both formats in the presence of 50 ng/mL either CSF1 or IL-34 whilst titrating in various concentrations of said antibodies (FIGS. 4A-4B and FIG. 5, respectively for each antibody format). The IC₅₀ values were determined from the titration curves.

As shown in Table 5, the IC₅₀ values determined for the inhibitory activity of the sdAbs against CSF1 ligand range from 0.5 to 4.0 nM. Clones Ab1 Parental, Ab1-9, Ab1-12, Ab1-18, and Ab-20 of this format had determined IC₅₀ values that were also in the low nanomolar range against IL-34.

TABLE 5
Inhibition Activity of sdAb by Macrophage Proliferation Assay

IC₅₀ (nM)

| Clone | CSF1 | IL-34 |
|---|---|---|
| Ab1 Parental | 2.0 | 2.2 |
| Ab1-9 | 2.3 | 3.6 |
| Ab1-12 | 4.0 | 3.6 |
| Ab1-18 | 2.3 | 4.2 |
| Ab1-20 | 2.6 | 3.6 |
| Ab2 Parental | 0.54 | N.D. |
| Ab2-10 | 1.9 | N.D. |
| Ab3 Parental | 1.2 | N.D. |

As shown in Table 6, the IC50 values of the sdAb monovalent Fc-fusion format clones were also in the low nanomolar against both CSF1 and IL-34.

TABLE 6
Inhibition Activity of sdAb Monovalent Fc-Fusion
Format by Macrophage Proliferation Assay

IC₅₀ (nM)

| Clone | CSF1 | IL-34 |
|---|---|---|
| Ab1 Parental | 6.8 | 7.5 |
| Ab2 Parental | 8.4 | 7.9 |
| Ab3 Parental | 12.4 | 5.3 |

### Example 3. Epitope Mapping Assay

This example demonstrates that exemplary anti-CSF1R sdAbs of the present invention bind to D5 of CSF1R.

### Methods for Epitope Mapping Assay

A CMDP chip (Carterra) was installed in the Carterra LSA (Carterra) and conditioned with 1×HBSTE (Carterra), glycine pH 2.0 (Carterra), 50 mM NaOH, 500 mM NaCl using 1×HBSTE running buffer. Post conditioning, the chip was activated with NHS and EDC, and AffiniPure goat anti-human IgG Fcγ fragment specific (Jackson ImmunoResearch labs) was coupled to the chip. The chip was then quenched with ethanolamine. Antibodies were diluted to 1 μg/mL in 1×HBSTE+0.1% BSA and 1 mL was dispensed into a 96-well plate. Antigen was diluted to 50 nM and 400 μL was dispensed into a 96-well plate. A kinetics capture experiment was performed with multiple cycles, the conditions of each cycle being identical. Antibody was captured to the chip for 5 minutes, antigen was associated for 5 minutes, dissociation in 1×HBSTE+0.1% BSA was performed for 10 minutes and finally regeneration with glycine pH 2.0 was performed for 30 seconds two times. All data was analyzed using Carterra kinetics software.

### Results of Epitope Mapping

The exemplary epitope mapping experiment using the Carterra-based protocol above was run against three samples: mouse CSF1R [m(D1D2D3D4D5)](SEQ ID NO: 160), mouse CSF1R with a humanized D5 domain [m(D1D2D3D4)h(D5)](SEQ ID NO: 161), and human CSF1R [h(D1D2D3D4D5)](SEQ ID NO: 159).

TABLE 7
Epitope of Exemplary Anti-CSF1R sdAbs

| Clone | Epitope |
|---|---|
| Ab1 | D5 |
| Ab2 | D5 |
| Ab3 | D5 |

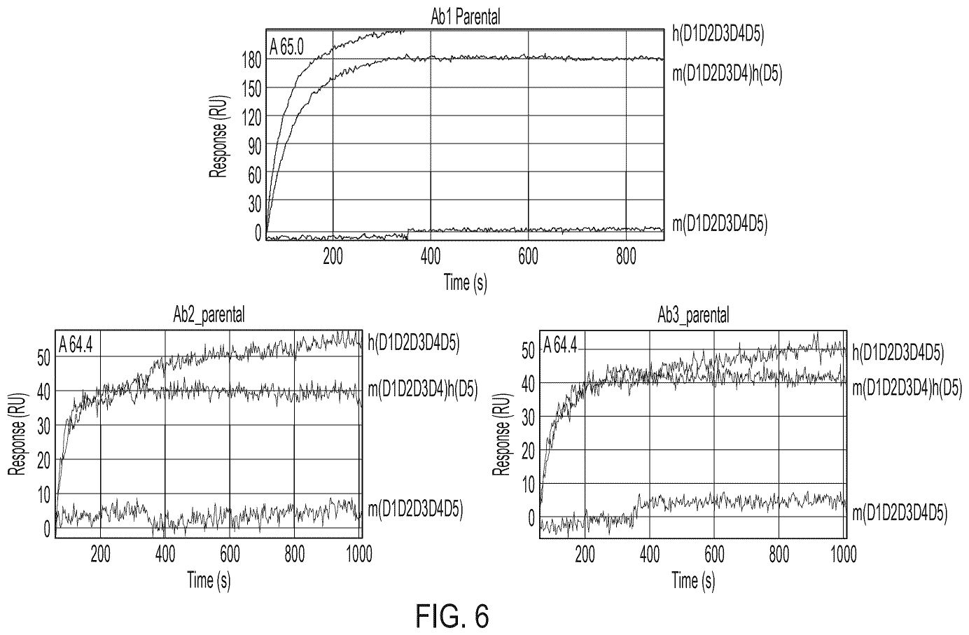

As shown in Table 7, Ab1, Ab2, and Ab3 bind to D5 of CSF1R. Furthermore, as shown in FIG. 6, the Parental clones of Ab1, Ab2 and Ab3 did not show an increase in response to the mouse CSF1R, but however exhibited an increase in response over time when in the presence of the mouse CSF1R with a humanized D5 domain and the human CSF1R. Likewise, the humanized antibodies such as Ab1_9, 12, 18 and 20, and Ab2_10 did not show an increase in response to the mouse CSF1R, but however exhibited an increase in response over time when in the presence of the mouse CSF1R with a humanized D5 domain and the human CSF1R. This serves as confirmation that the epitope that the sdAbs of Parental Ab1, Ab2 and Ab3 and their humanized sdAbs bind to is the D5 domain of the human CSF1R receptor.

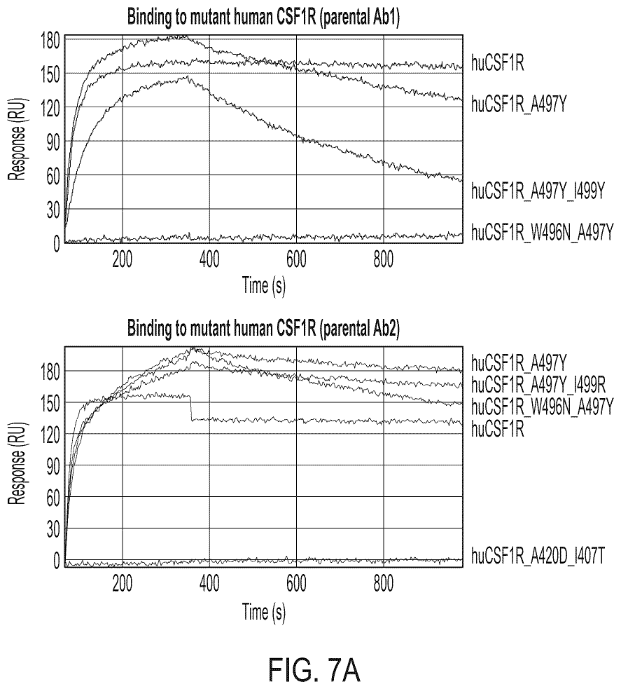

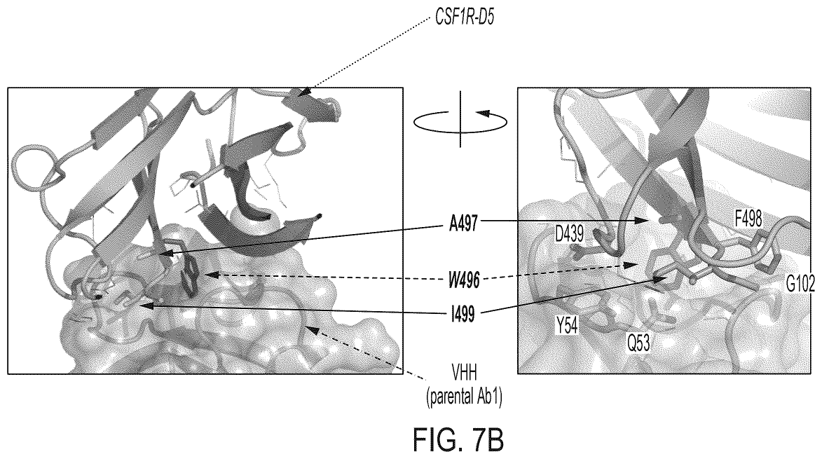

To further assess the core residues in D5 that the exemplary anti-CSF1R sdAbs bind to, point mutations were made within D5 of the human CSF1R receptor, and binding of the sdAbs were measured. The exemplary anti-CSF1R sdAbs used in this study were the Ab1 and Ab2 sdAbs. The tested point mutations include A497Y, A497Y/I499R, W496N/A497Y, and A420D/I407T. As shown in FIG. 7A, mutations at residues W496 and A497 abolished the binding of Ab1 to CSF1R, and the mutations at residues A420 and I407 abolished the binding of Ab2 to CSF1R. Additionally, the mutations at A497 and 1499 significantly decreased binding of Ab1 to CSF1R. Overall, the data suggests that the residues W496 and A497 are core epitope residues for Ab1, and the residues A420 and I407 are core epitope residues for Ab2. Cryo-EM Epitope Mapping

Cryo-electron microscopy (cryo-EM) was used to determine the structure of the complex between Ab1 and CSF1R_D5 domain. The complex was obtained by mixing purified CSF1R_D5 domain with Ab1. The complex solution was applied to EM grid and rapidly frozen with liquid ethane. The dataset was collected using a 300 kV transmission electron microscope. Images were processed using CryoSPARC™, and 2D/3D classification followed by 3D reconstruction produced a density map. The final map, shown in FIG. 7B, reached an overall global resolution of 3.0 Å, and the Ab1 was confirmed to bind the core epitope residues of W496, A497, and 1499 on D5 domain of CSF1R. In addition to the three core epitope residues, the following residues are also considered to contribute to Ab1 binding: P402, E403, V404, S405, V406, I407, W408, L417, A420, A421, S422, H437, T438, D439, R440, K465, T467, G494, S495, F498, and P500.

### Example 4. In Vivo Activity of Anti-CSF1R Antibodies in a Dry AMD Model

This example assesses the pharmacological effects of an exemplary anti-CSF1R antibody in a dry age-related macular degeneration (AMD) mouse model. Sodium iodate-induced retinal pigment epithelium (RPE) damage in mice serves as a model for dry AMD. The objective of this study was to ascertain whether the exemplary anti-CSF1R antibodies could inhibit the reduction of retinal marker gene expression.

In this particular study, an anti-mouse CSF1R antibody in an IgG format was used.

### Methods for Creating the Mouse Model of Sodium Iodate-Induced RPE Damage

Prior to the administration of sodium iodate to create the dry AMD mouse model used in this study, C57BL/6 mice were anesthetized with a combination of medetomidine hydrochloride, midazolam, and butorphanol tartrate. Following anesthetization, a 2.2 mg/mL solution of sodium iodate dissolved in water was intravenously administered at a dose of 10 mL/kg. Immediately following the administration of the sodium iodate solution, the animals were awakened with an injection of atipamezole hydrochloride.

### Antibody Administration

The intravitreal injection of the antibody into the mice was performed under isoflurane anesthesia at a concentration of 15 or 50 pg/eye (1 μL/eye) after pupil dilation. The injections were carried out 3 days before the creation of the sodium iodate-induced RPE damage model.

### Evaluation of Retinal Marker Gene Expression

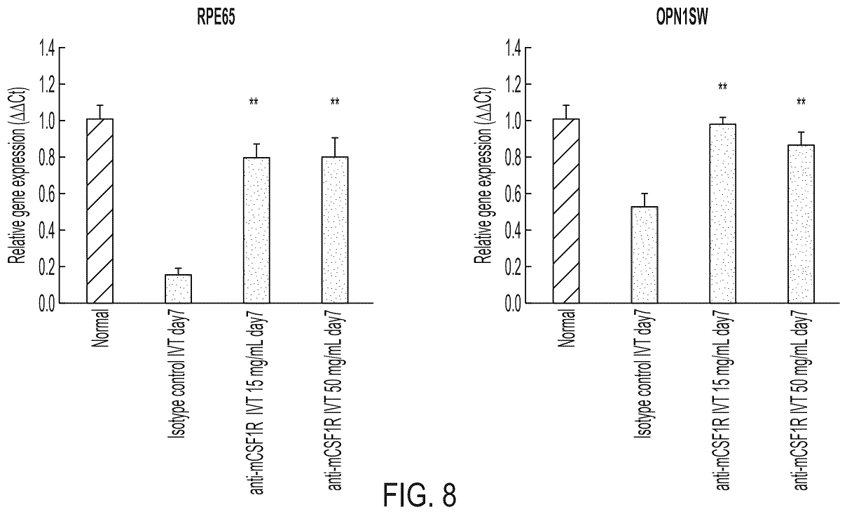

Seven days after the creation of the sodium iodate-induced RPE damage model, the retina, RPE, and choroid were collected. The collected samples were analyzed for the expression levels of retinal marker genes (RPE65, OPN1SW) using quantitative PCR. The administration of the antibody significantly inhibited the reduction in the expression levels of the RPE marker gene RPE65 and the cone cell marker gene OPN1SW (FIG. 8). These results confirm that the anti-CSF1R antibodies inhibit the reduction of retinal marker gene expression in the sodium iodate-induced RPE damage model, which serves as a model for dry AMD.

### EQUIVALENTS AND SCOPE

Those skilled in the art will recognize, or be able to ascertain using no more than routine experimentation, many equivalents to the specific embodiments of the disclosure described herein. The scope of the present disclosure is not intended to be limited to the above description, but rather is as set forth in the following claims:

1. A single domain antibody (sdAb) that binds to colony stimulating factor 1 receptor (CSF1R), wherein the sdAb comprises
a. a CDR1 comprising an amino acid sequence that is identical to or differs by no more than 2 amino acid residues from GGTFSYY (SEQ ID NO: 106), a CDR2 comprising an amino acid sequence that is identical to or differs by no more than 2 amino acid residues from SQYGSN (SEQ ID NO: 109), and a CDR3 comprising an amino acid sequence that is identical to or differs by no more than 2 amino acid residues from LRSGSDWRTIRDYDN (SEQ ID NO: 112); or
b. a CDR1 comprising an amino acid sequence that is identical to or differs by no more than 2 amino acid residues from AASGGTFSYYNMG (SEQ ID NO: 124), a CDR2 comprising an amino acid sequence that is identical to or differs by no more than 2 amino acid residues from VISQYGSNTY (SEQ ID NO: 127), and a CDR3 comprising an amino acid sequence that is identical to or differs by no more than 2 amino acid residues from ATLRSGSDWRTIRDYDN (SEQ ID NO: 130); or
c. a CDR1 comprising an amino acid sequence that is identical to or differs by no more than 2 amino acid residues from GGTFSYYN (SEQ ID NO: 115), a CDR2 comprising an amino acid sequence that is identical to or differs by no more than 2 amino acid residues from ISQYGSNT (SEQ ID NO: 118), and a CDR3 comprising an amino acid sequence that is identical to or differs by no more than 2 amino acid residues from ATLRSGSDWRTIRDYDN (SEQ ID NO: 121); or
d. a CDR1 comprising an amino acid sequence that is identical to or differs by no more than 2 amino acid residues from YYNMG (SEQ ID NO: 133), a CDR2 comprising an amino acid sequence that is identical to or differs by no more than 2 amino acid residues from VISQYGSNTYYADSVKG (SEQ ID NO: 136), and a CDR3 comprising an amino acid sequence that is identical to or differs by no more than 2 amino acid residues from LRSGSDWRTIRDYDN (SEQ ID NO: 139); or
e. a CDR1 comprising an amino acid sequence that is identical to or differs by no more than 2 amino acid residues from GDIFSFN (SEQ ID NO: 107), a CDR2 comprising an amino acid sequence that is identical to or differs by no more than 2 amino acid residues from APGGN (SEQ ID NO: 110), and a CDR3 comprising an amino acid sequence that is identical to or differs by no more than 2 amino acid residues from WPFSRDS (SEQ ID NO: 113); or
f. a CDR1 comprising an amino acid sequence that is identical to or differs by no more than 2 amino acid residues from AASGDIFSFNVMG (SEQ ID NO: 125), a CDR2 comprising an amino acid sequence that is identical to or differs by no more than 2 amino acid residues from TIAPGGNTN (SEQ ID NO: 156), and a CDR3 comprising an amino acid sequence that is identical to or differs by no more than 2 amino acid residues from NRWPFSRDS (SEQ ID NO: 131); or
g. a CDR1 comprising an amino acid sequence that is identical to or differs by no more than 2 amino acid residues from AASGDIFSFNVMG (SEQ ID NO: 125), a CDR2 comprising an amino acid sequence that is identical to or differs by no more than 2 amino acid residues from VIAPGGNTY (SEQ ID NO: 128), and a CDR3 comprising an amino acid sequence that is identical to or differs by no more than 2 amino acid residues from NRWPFSRDS (SEQ ID NO: 131); or
h. a CDR1 comprising an amino acid sequence that is identical to or differs by no more than 2 amino acid residues from GDIFSFNV (SEQ ID NO: 116), a CDR2 comprising an amino acid sequence that is identical to or differs by no more than 2 amino acid residues from IAPGGNT (SEQ ID NO: 119), and a CDR3 comprising an amino acid sequence that is identical to or differs by no more than 2 amino acid residues from NRWPFSRDS (SEQ ID NO: 122); or
i. a CDR1 comprising an amino acid sequence that is identical to or differs by no more than 2 amino acid residues from FNVMG (SEQ ID NO: 134), a CDR2 comprising an amino acid sequence that is identical to or differs by no more than 2 amino acid residues from TIAPGGNTNYFDSARG (SEQ ID NO: 157), and a CDR3 comprising an amino acid sequence that is identical to or differs by no more than 2 amino acid residues from WPFSRDS (SEQ ID NO: 140); or
j. a CDR1 comprising an amino acid sequence that is identical to or differs by no more than 2 amino acid residues from FNVMG (SEQ ID NO: 134), a CDR2 comprising an amino acid sequence that is identical to or differs by no more than 2 amino acid residues from VIAPGGNTYYADSVKG (SEQ ID NO: 137), and a CDR3 comprising an amino acid sequence that is identical to or differs by no more than 2 amino acid residues from WPFSRDS (SEQ ID NO: 140); or
k. a CDR1 comprising an amino acid sequence that is identical to or differs by no more than 2 amino acid residues from GMAFGSS (SEQ ID NO: 108), a CDR2 comprising an amino acid sequence that is identical to or differs by no more than 2 amino acid residues from TSAGY (SEQ ID NO: 111), and a CDR3 comprising an amino acid sequence that is identical to or differs by no more than 2 amino acid residues from GPFHSTAYST (SEQ ID NO: 114); or
l. a CDR1 comprising an amino acid sequence that is identical to or differs by no more than 2 amino acid residues from VASGMAFGSSPMN (SEQ ID NO: 126), a CDR2 comprising an amino acid sequence that is identical to or differs by no more than 2 amino acid residues from GVTSAGYAY (SEQ ID NO: 129), and a CDR3 comprising an amino acid sequence that is identical to or differs by no more than 2 amino acid residues from ARGPFHSTAYST (SEQ ID NO: 132); or
m. a CDR1 comprising an amino acid sequence that is identical to or differs by no more than 2 amino acid residues from GMAFGSSP (SEQ ID NO: 117), a CDR2 comprising an amino acid sequence that is identical to or differs by no more than 2 amino acid residues from VTSAGYA (SEQ ID NO: 120), and a CDR3 comprising an amino acid sequence that is identical to or differs by no more than 2 amino acid residues from ARGPFHSTAYST (SEQ ID NO: 123); or
n. a CDR1 comprising an amino acid sequence that is identical to or differs by no more than 2 amino acid residues from SSPMN (SEQ ID NO: 135), a CDR2 comprising an amino acid sequence that is identical to or differs by no more than 2 amino acid residues from GVTSAGYAYYADWVKG (SEQ ID NO: 138), and a CDR3 comprising an amino acid sequence that is identical to or differs by no more than 2 amino acid residues from GPFHSTAYST (SEQ ID NO: 141).

2. The sdAb of claim 1, wherein the sdAb comprises,
a. a CDR1 comprising an amino acid sequence of GGTFSYY (SEQ ID NO: 106), a CDR2 comprising an amino acid sequence of SQYGSN (SEQ ID NO: 109), and a CDR3 comprising an amino acid sequence of LRSGSDWRTIRDYDN (SEQ ID NO: 112); or
b. a CDR1 comprising an amino acid sequence of AASGGTFSYYNMG (SEQ ID NO: 124), a CDR2 comprising an amino acid sequence of VISQYGSNTY (SEQ ID NO: 127), and a CDR3 comprising an amino acid sequence of ATLRSGSDWRTIRDYDN (SEQ ID NO: 130); or
c. a CDR1 comprising an amino acid sequence of GGTFSYYN (SEQ ID NO: 115), a CDR2 comprising an amino acid sequence of ISQYGSNT (SEQ ID NO: 118), and a CDR3 comprising an amino acid sequence of ATLRSGSDWRTIRDYDN (SEQ ID NO: 121); or
d. a CDR1 comprising an amino acid sequence of YYNMG (SEQ ID NO: 133), a CDR2 comprising an amino acid sequence of VISQYGSNTYYADSVKG (SEQ ID NO: 136), and a CDR3 comprising an amino acid sequence of LRSGSDWRTIRDYDN (SEQ ID NO: 139); or
e. a CDR1 comprising an amino acid sequence of GDIFSFN (SEQ ID NO: 107), a CDR2 comprising an amino acid sequence of APGGN (SEQ ID NO: 110), and a CDR3 comprising an amino acid sequence of WPFSRDS (SEQ ID NO: 113); or
f. a CDR1 comprising an amino acid sequence of AASGDIFSFNVMG (SEQ ID NO: 125), a CDR2 comprising an amino acid sequence of TIAPGGNTN (SEQ ID NO: 156), and a CDR3 comprising an amino acid sequence of NRWPFSRDS (SEQ ID NO: 131); or
g. a CDR1 comprising an amino acid sequence of AASGDIFSFNVMG (SEQ ID NO: 125), a CDR2 comprising an amino acid sequence of VIAPGGNTY (SEQ ID NO: 128), and a CDR3 comprising an amino acid sequence of NRWPFSRDS (SEQ ID NO: 131); or
h. a CDR1 comprising an amino acid sequence of GDIFSFNV (SEQ ID NO: 116), a CDR2 comprising an amino acid sequence of IAPGGNT (SEQ ID NO: 119), and a CDR3 comprising an amino acid sequence of NRWPFSRDS (SEQ ID NO: 122); or
i. a CDR1 comprising an amino acid sequence of FNVMG (SEQ ID NO: 134), a CDR2 comprising an amino acid sequence of TIAPGGNTNYFDSARG (SEQ ID NO: 157), and a CDR3 comprising an amino acid sequence of WPFSRDS (SEQ ID NO: 140); or
j. a CDR1 comprising an amino acid sequence of FNVMG (SEQ ID NO: 134), a CDR2 comprising an amino acid sequence of VIAPGGNTYYADSVKG (SEQ ID NO: 137), and a CDR3 comprising an amino acid sequence of WPFSRDS (SEQ ID NO: 140); or
k. a CDR1 comprising an amino acid sequence of GMAFGSS (SEQ ID NO: 108), a CDR2 comprising an amino acid sequence of TSAGY (SEQ ID NO: 111), and a CDR3 comprising an amino acid sequence of GPFHSTAYST (SEQ ID NO: 114); or
l. a CDR1 comprising an amino acid sequence of VASGMAFGSSPMN (SEQ ID NO: 126), a CDR2 comprising an amino acid sequence of GVTSAGYAY (SEQ ID NO: 129), and a CDR3 comprising an amino acid sequence of ARGPFHSTAYST (SEQ ID NO: 132); or
m. a CDR1 comprising an amino acid sequence of GMAFGSSP (SEQ ID NO: 117), a CDR2 comprising an amino acid sequence of VTSAGYA (SEQ ID NO: 120), and a CDR3 comprising an amino acid sequence of ARGPFHSTAYST (SEQ ID NO: 123); or
n. a CDR1 comprising an amino acid sequence of SSPMN (SEQ ID NO: 135), a CDR2 comprising an amino acid sequence of GVTSAGYAYYADWVKG (SEQ ID NO: 138), and a CDR3 comprising an amino acid sequence of GPFHSTAYST (SEQ ID NO: 141).

3. The sdAb of claim 1, wherein the sdAb comprises an amino acid sequence at least 85%, 90%, 95%, 96%, 97%, 98%, or 99% identical to any one of SEQ ID NOs: 1-3 or 101-105.

4. (canceled)

5. A single domain antibody (sdAb) that binds to colony stimulating factor 1 receptor (CSF1R), wherein the sdAb binds to D5 of CSF1R, corresponding to residues 402-502 of SEQ ID NO: 142.

6. The sdAb of claim 5, wherein the sdAb binds to contiguous residues of D5.

7-19. (canceled)

20. The sdAb of claim 1, wherein the sdAb is fused to an Fc region, and wherein the Fc region is derived from IgG1, IgG2, IgG3, or IgG4.

21. (canceled)

22. The sdAb of claim 20, wherein the Fc region is modified.

23. A nucleic acid encoding the sdAb of claim 1.

24. A vector comprising the nucleic acid of claim 23.

25. The vector of claim 24, wherein the vector is a viral vector.

26. The vector of claim 25, wherein the viral vector is an adeno-associated virus (AAV) vector.

27. (canceled)

28. The AAV vector of claim 26, wherein the vector is selected from the group consisting of AAV1, AAV2, AAV3, AAV4, AAV5, AAV6, AAV7, AAV8, AAV9, AAV10, AAV11, AAV12, AAV13.

29. A liquid nanoparticle (LNP) comprising the vector of claim 24.

30. A host cell comprising the vector of claim 24.

31-32. (canceled)

33. A pharmaceutical composition comprising the sdAb of claim 1, and one or more physiologically acceptable carriers, excipients, or diluents.

34. A method of treating a disease or disorder associated with CSF1R in a subject in need thereof, comprising administering to the subject the sdAb of claim 1.

35. A method of treating age-related macular degeneration (AMD) in a subject in need thereof, comprising administering to the subject the sdAb of claim 1.

36-45. (canceled)

46. A single domain antibody (sdAb) comprising means for binding to colony stimulating factor 1 receptor (CSF1R).

47. The single domain antibody (sdAb) of claim 46, wherein the sdAb binds to D5 of colony stimulating factor 1 receptor (CSF1R), corresponding to residues 402-502 of SEQ ID NO: 142.

48-57. (canceled)

58. A single domain antibody (sdAb) that binds to colony stimulating factor 1 receptor (CSF1R), wherein the sdAb comprises
a. a CDR1 comprising a consensus sequence of GX₁TFSX₂YN (SEQ ID NO: 147), wherein X₁ comprises G, L, or F, and X₂ comprises S or Y; a CDR2 comprising an amino acid sequence of ISQYGSNT (SEQ ID NO: 150); and a CDR3 comprising a consensus sequence of TLRSGSDWRTX₃RDYDNW (SEQ ID NO: 153), wherein X₃ comprises I or M; or
b. a CDR1 comprising a consensus sequence of GX₄X₅FGSX₆ (SEQ ID NO: 148), wherein X₄ comprises F, M, or L, X₅ comprises T or A, and X₆ comprises T or S; a CDR2 comprising a consensus sequence of X₇TSX₈GYX₉ (SEQ ID NO: 151), wherein X₇ comprises V or T, X₈ comprises S, A, or G, and X₉ comprises S, A, or T; and a CDR3 comprising a consensus sequence of RGPFHSX₁₀AYX₁₁TR (SEQ ID NO: 154), wherein X₁₀ comprises T or I, and X₁₁ comprises A or S; or
c. a CDR1 comprising a consensus sequence of GX₂₁IFX₂₂X₂₃X₂₄V (SEQ ID NO: 149), wherein X₂₁ comprises D or N, X₂₂ comprises R or S, X₂₃ comprises F or S, and X₂₄ comprises T or N; a CDR2 comprising a consensus sequence of IX₂₅X₂₆GGX₂₇T (SEQ ID NO: 152), wherein X₂₅ comprises T, A, or S, X₂₆ comprises S, P, or T, and X₂₇ comprises R, N, or K; and a CDR3 comprising a consensus sequence of X₂₈LPFX₂₉RDS (SEQ ID NO: 155), wherein X₂₈ comprises R, T, or S, and X₂₉ comprises R or S.

59-60. (canceled)
